(19) United States
(12) Patent Application Publication
(10) Pub. No.: US 2007/0154882 A1
(43) Pub. Date: Jul. 5, 2007

(54) Beta-polypeptides that inhibit cytomegalovirus infection

(76) Inventors: Teresa Compton, Winchester, MA (US); Samuel H. Gellman, Madison, WI (US); Emily P. English, Madison, WI (US); Robert S. Chumanov, Middleton, WI (US)

Correspondence Address: DEWITT ROSS & STEVENS S.C., 8000 EXCELSIOR DR, SUITE 401, MADISON, WI, 53717-1914, US

(21) Appl. No.: 11/372,840

(22) Filed: Mar. 10, 2006

Related U.S. Application Data
(60) Provisional application No. 60/660,485, filed on Mar. 10, 2005.

(51) Int. Cl. C12Q 1/70

(52) U.S. Cl. 435/5

(57) ABSTRACT

Disclosed are beta-polypeptides that mimic the coiled-coil regions of gB and gH by display of the key hydrophobic residues for coiled-coil packing along one face of beta-polypeptide 12-helix. The most potent inhibitor blocks infection of CMV with an IC₅₀ of approximately 20 m.

### CROSS-REFERENCE TO RELATED APPLICATIONS

Priority is hereby claimed to provisional application Ser. No. 60/660,485, filed Mar. 10, 2005, and incorporated herein by reference.

### FEDERAL FUNDING STATEMENT

This invention was made with United States government support awarded by the following agency: NIH Grants GM056414 and AI034998. The United States has certain rights in this invention.

### BACKGROUND OF THE INVENTION

Human cytomegalovirus (HCMV) is a member of the medically significant Herpesviridae family of viruses, a family divided into three subfamilies: alpha-, beta- and gamma herpesviruses. Herpesviruses establish a life-long relationship with their hosts and can manifest disease in an opportunistic manner. HCMV is the most common viral cause of congenital birth defects and is responsible for significant morbidity and mortality in immuno-compromised patients, including AIDS patients and organ transplant recipients. See Ljungman, P. Cytomegalovirus infections in transplant patients. Scand J Infect Dis Suppl 100, 59-63 (1996); and Ramsay, M. E., Miller, E. & Peckham, C. S. Outcome of confirmed symptomatic congenital cytomegalovirus infection. Arch Dis Child 66, 1068-9 (1991). A notable feature of HCMV pathogenesis is its exceptionally broad tissue tropism. HCMV is capable of manifesting disease in most organ systems and tissue types, which directly correlates with its ability to infect fibroblasts, endothelial cells, epithelial cells, monocytes/macrophages, smooth muscle cells, stromal cells, neuronal cells, neutrophils, and hepatocytes. In vitro entry into target cells is equally promiscuous, as HCMV is able to bind, penetrate and initiate replication in all tested vertebrate cell types. See D. M., Cooper, N. R. & Compton, T. Expression of a human cytomegalovirus receptor correlates with infectibility of cells. J Virol 65, 3114-21 (1991). Recently, epidermal growth factor receptor (EGFR) was identified as a cellular receptor for HCMV whose expression correlated with the ability of the virus to initiate gene expression. Wang, X., Huong, S. M., Chiu, M. L., Raab-Traub, N. & Huang, E. S. Epidermal growth factor receptor is a cellular receptor for human cytomegalovirus. Nature 424, 456-61 (2003). However, EGFR is not expressed on several HCMV permissive cells, such as hematopoetic cell types and therefore other receptors must exist.

Researchers have established that three viral glycoproteins, gB, gH, and gL, mediate viral fusion with the cell membrane. Among these three, it is generally accepted that viral glycoprotein B (gB) is required for virus entry and fusion throughout the Herpesviridae family. Glycoprotein B is a critical member of the conserved basic fusion machinery. Spear, P.G. & Longnecker, R. Herpesvirus entry: an update. J Virol 77, 10179-85 (2003). During virus entry, HCMV induces cellular morphological changes and signaling cascades consistent with engagement of cellular integrins; however, HCMV structural proteins do not possess the widely used RGD integrin binding motif. At present, no crystal or NMR structure data on gB or gH has been reported in the scientific literature.

Viral fusion is generally thought to proceed by a three-step process. A first activation step involves the extension of a coiled-coil trimer from the virion to the cell membrane of the cell to be infected. This “fusion” peptide is inserted into the cell membrane. The second step involves a rearrangement of the carboxy-terminal of the coiled-coil fusion peptide. The third step involves a linking mechanism that firmly attaches the virion to the cell membrane. If any of these fusion steps can be disrupted, the ability for a virion to fuse to the cell membrane would likewise be disrupted.

The compounds, compositions, and methods described herein include or utilize oligomers and polymers comprised of cyclically-constrained beta-amino acids. Much work on beta-amino acids and peptides synthesized therefrom has been performed by two groups of scientists, a first group led by Samuel Gellman at the University of Wisconsin-Madison, and a second group led by Dieter Seebach in Zurich, Switzerland. For example, Dado and Gellman (1994) J. Am. Chem. Soc. 116:1054-1062 describe intramolecular hydrogen bonding in derivatives of beta-alanine and gamma-amino butyric acid. This paper postulates that beta-peptides will fold in manners similar to alpha-amino acid polymers if intramolecular hydrogen bonding between nearest neighbor amide groups on the polymer backbone is not favored. See also Schmitt, Margaret A.; Weisblum, Bernard; Gellman, Samuel H. “Unexpected Relationships between Structure and Function in Alpha, Beta-Peptides: Antimicrobial Foldamers with Heterogeneous Backbones.” J Am Chem Soc (2004), 126(22), 6848-6849. In the patent literature, see U.S. Pat. Nos. 6,958,384; 6,914,048; 6,727,368; 6,710,186; 6,683,154; 6,613,876; and 6,060,585, all to Gellman et al.

From the Seebach group, see, for example, Seebach et al. (1996) Helv. Chim. Acta. 79:913-941; and Seebach et al. (1996) Helv. Chim. Acta. 79:2043-2066. In the first of these two papers Seebach et al. describe the synthesis and characterization of a beta-hexapeptide, namely (H——HVal— —HAla— —HLeu)₂-OH. Interestingly, this paper specifically notes that prior art reports on the structure of beta-peptides have been contradictory and “partially controversial.” In the second paper, Seebach et al. explore the secondary structure of the above-noted beta-hexapeptide and the effects of residue variation on the secondary structure. See also U.S. Pat. No. 6,617,425, to Seebach.

### SUMMARY OF THE INVENTION

Because the viral glycoproteins gB, gH, and gL are known to mediate viral fusion, the present inventors sought to identify compounds that inhibit the action of these glycoproteins, thereby inhibiting the ability of HCMV to infect cells.

Thus, the invention is directed to a method for inhibiting viral entry into an animal host cell (including human cells) and a corresponding pharmaceutical composition for inhibiting viral entry into an animal host cell. The method comprising administering to the host cell a viral fusion-inhibiting amount of a compound capable of inhibiting viral entry into the host cell. In the preferred embodiment, the compound is selected from the group consisting of beta-amino acid-containing polypeptides comprising eight (8) or more residues, wherein at least one of the residues is a beta-amino acid residue wherein the alpha and beta carbons are cyclically constrained, and pharmaceutically suitable salts thereof.

In one version of the invention, at least three (3) of the residues are beta-amino acid residues wherein the alpha and beta carbons are cyclically constrained. In another version, at least five (5) of the residues are beta-amino acid residues wherein the alpha and beta carbons are cyclically constrained. The compound may be selected from the group consisting of: ERP-I-301, EPE-II-219, EPE-II-221, EPE-11-223, EPE-II-227, EPE-II-225, EPE-II-229, EPE-II-233, EPE-II-231, EPE-II-235, EPE-II-237, EPE-II-239, EPE-II-241, EPE-II-243, EPE-II-247, EPE-II-245, EPE III-137, EPE-III-139, EPE-III-141, EPE-III-143, EPE-III-145, EPE-III-147, and pharmaceutically suitable salts thereof.

In a preferred version of the invention, the beta-amino acid-containing polypeptides comprise eight (8) to thirteen (13) residues, all of which are beta-amino acid residues. As noted earlier, at least one of the residues is a beta-amino acid residue wherein the alpha and beta carbons are cyclically constrained. In a related version of the invention, the polypeptide comprises at least one alpha-amino acid residue, and wherein at least one other of the residues is a cyclically constrained beta-amino acid residue. Where the compound contains both alpha-amino acid residues and beta-amino acid residues, it is preferably selected from the group consisting of: 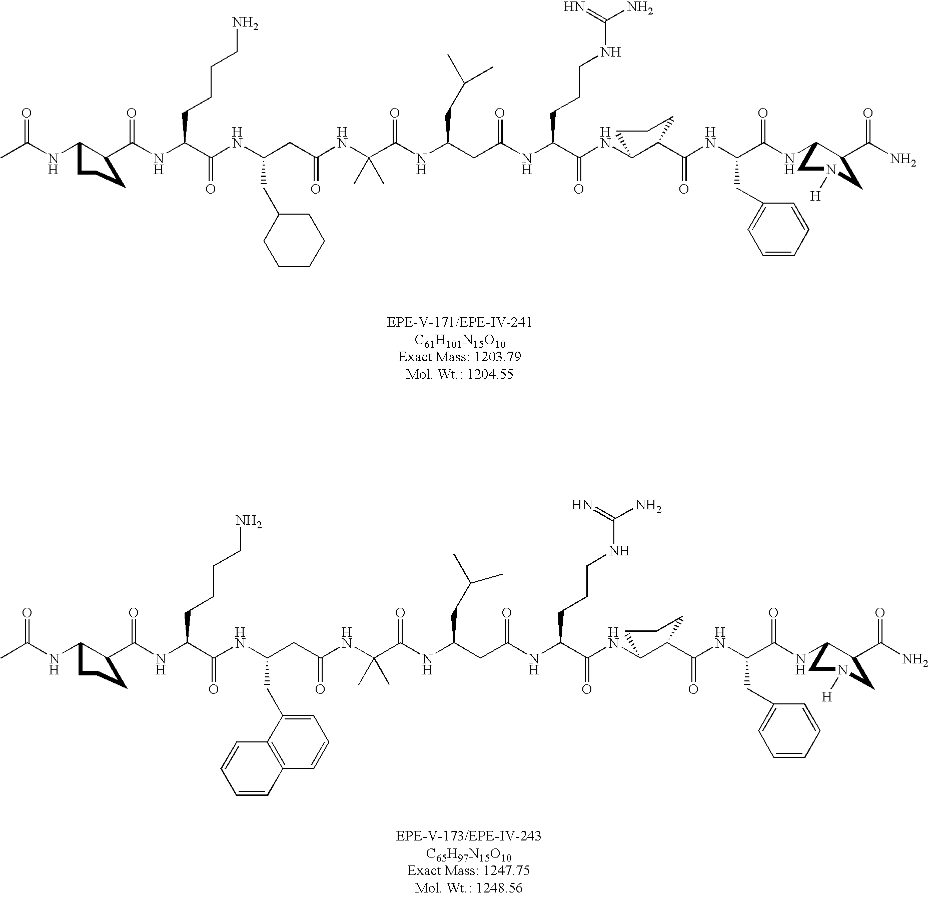
and pharmaceutically suitable salts thereof.

As described herein, the compound may be administered in combination with a pharmaceutically suitable carrier suitable for a delivery route selected from the group consisting of oral, parenteral, topical, subcutaneous, transdermal, intramuscular, intravenous, intra-arterial, buccal, and rectal.

The principal advantage and utility of the present invention is that it provides a means to inhibit viral infection of animal cells, including human cells, using compounds (beta-polypeptides) that are far more resistant to enzymatic degradation than are natural alpha-amino acids.

### BRIEF DESCRIPTION OF THE DRAWINGS

FIG. 1 is a histogram depicting the ability of beta-polypeptide inhibitors according to the present invention to inhibit human cytomegalovirus (HCMV) infection of normal human dermal fibroblast (NHDF) cells. Each compound was administered at a concentration of 10 M. The entire height of each bar represents the percentage of live cells remaining after being treated with each compound; the area below the horizontal line in each bar represents the percentage of GFP-positive cells (an indication of how many cells were infected; see the Examples). The compound labeled “inhibitor” in the figure is compound EPE-III-139.

FIG. 2 is a histogram generated in the same fashion as the histogram shown in FIG. 1, with the exception that each compound was administered at a concentration of 10 M.

### DETAILED DESCRIPTION OF THE INVENTION

The present invention is directed to methods and compositions for inhibiting fusion of a virus, specifically a HCMV (e.g., herpesviruses) into the cellular membrane of a host cell. This is done by contacting the host cell with a polypeptide comprising one or cyclically-constrained beta-amino acid residues (referred to herein as “beta-polypeptides) or comprising one or more alpha-amino acid residues and one or more cyclically-constrained beta-amino acid residues. While not being limited to any specific underlying biological phenomenon, it is believed that the beta-polypeptides disclosed herein are inhibitors of one or more of the gB, gH, and/or gL viral glycoproteins that are required for cell fusion. By inhibiting the function of one or more of these glycoproteins, the beta-polypeptides disclosed herein are able to inhibit infection of treated cells by HCMV.

As used herein, the term “alpha-amino acid” refers to any alpha amino acid, natural or unnatural, without limitation, and derivatives thereof, such as N-alkylated alpha-amino acids, etc. By definition, an alpha-amino acid is an amino acid having a single carbon atom disposed between the carboxyl terminus and the amino terminus. Thus, the term alpha amino acid as used herein does not encompass beta amino acids.

As used herein, the term “beta-polypeptide” refers to any beta-polypeptide of 8 or more residues, wherein in at least one of the residues the alpha and beta carbons are cyclically constrained, as well as pharmaceutically suitable salts thereof. Beta-polypeptides for use in the present invention can be synthesized, isolated, purified, and characterized as explained in Gellman et al., U.S. Pat. No. 6,613,876, titled “Beta-Polypeptide Foldamers of Well-Defined Secondary Structure;” Gellman et al., U.S. Pat. No. 6,683,154, titled “Antimicrobial Compositions Containing Beta-Amino Acid Oligomers;” Gellman et al., U.S. Pat. No. 6,710,186, titled “Oligomers and Polymers of Di-Substituted Cyclic Imino Carboxylic Acids;” and Gellman et al., U.S. Pat. No. 6,727,368, titled “Oligomers and Polymers of Cyclic Imino Carboxylic Acids,” all of which are incorporated herein.

The term includes all (D) and (L) stereoisomers of such amino acids when the structure of the amino acid admits of stereoisomeric forms, as well as C-terminal or N-terminal protected amino acid derivatives (e.g., modified with an N-terminal or C-terminal protecting group such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminopimelic acid, ornithine, or diaminobutyric acid). As used herein, the term “protecting group” in general, and “amino-terminus protecting group” and “carboxy-terminus protecting group” in particular, refer to any chemical moiety capable of addition to and (optionally) removal from a reactive site (an amino group and a carboxy group, respectively, in the particular instance) to allow manipulation of a chemical entity at sites other than the reactive site. Protecting groups, and the manner in which they are introduced and removed are described, for example, in “Protective Groups in Organic Chemistry,” Plenum Press, London, N.Y. 1973; and in “Methoden der organischen Chemie,” Houben-Weyl, 4th edition, Vol. 15/1, Georg-Thieme-Verlag, Stuttgart 1974; and in Theodora W. Greene, “Protective Groups in Organic Synthesis,” John Wiley & Sons, New York 1981. A characteristic of many protecting groups is that they can be removed readily, i.e., without the occurrence of undesired secondary reactions, for example by solvolysis, reduction, photolysis or alternatively under physiological conditions.

A host of protecting groups and how to use them are known in the art, and therefore they shall not be described in any detail herein. An illustrative, non-limiting list of protecting groups includes methyl, formyl, ethyl, acetyl, t-butyl, benzyl, trifluoroacetyl, t-butoxycarbonyl, benzoyl, 4-methylbenzyl, benzyloxymethyl, 4-nitrophenyl, benzyloxycarbonyl, 2-nitrobenzoyl, 2-nitrophenylsulphenyl, 4-toluenesulphonyl, pentafluorophenyl, diphenylmethyl, 2-chlorobenzyloxycarbonyl, 2,4,5-trichlorophenyl, 2-bromobenzyloxycarbonyl, 9-fluorenylmethyloxycarbonyl, triphenylmethyl, and 2,2,5,7,8-pentamethyl-chroman-6-sulphonyl. The terms “amino-terminus protecting group” and “carboxy-terminus protecting group” as used herein are explicitly synonymous with such terms as “N-terminal capping group” and “C-terminal capping group,” respectively. A host of suitable protecting and capping groups, in addition to those described above, are known in the art. For discussions of various different types of amino- and carboxy-protecting groups, see, for example, U.S. Pat. Nos. 5,256,549; 5,221,736; 5,521,184; and 5,049,656.

In one embodiment, the invention provides a method of inhibiting the entry of herpesviruses into a host cell by introducing or administering an effective amount of a beta-polypeptide. A herpesvirus infection is exemplary. As used herein, the term “host cell” refers to an animal cell, suitably a human cell. The beta-polypeptide of the invention may be mixed with a pharmaceutically acceptable, nontoxic carrier. Also, it is within the scope of the invention that the beta-polypeptide may be linked to another moiety, such as an internalizing peptide, an accessory peptide or a transport moiety. The agent may be a peptidomimetic, especially for the viral glycopeptides gB, gH, gL.

The beta-polypeptides of the present invention may be administered by any of a variety of routes depending upon the specific end use. These agents may be administered directly to virus infected cells, suitably CMV-infected cells. Direct delivery of such polypeptide therapeutics may be facilitated by formulation of the compound in any pharmaceutically suitable dosage form, e.g., for delivery orally, parenterally, intratumorally, peritumorally, interlesionally, intravenously, intramuscularly, periolesionally, rectally, or topically to exert local therapeutic effects. Applicants envision that about 50 to 350 mg of peptide, preferably about 25 to 500 mg, and more preferably still about 10 to 1,000 mg (daily) is a suitable dose to be administered subcutaneously (in one or more discrete administrations per day) to a virus-infected subject, although amounts above and below this dosage range are part of the invention.

The most suitable route in any given case will depend upon the ailment being treated, the particular type of beta-polypeptide being administered, the subject involved, and the judgment of the medical practitioner. An agent of the invention may also be administered by means of controlled-release, depot implant or injectable formulations. The exact dose and regimen for administration of these agents will necessarily be dependent upon the needs of the individual subject being treated, the type of treatment, the degree of affliction or need, and the judgment of the medical practitioner. In general, parenteral administration requires lower dosage than other methods of administration (e.g., topical), which are more dependent upon absorption.

The compounds described herein being effective to inhibit the viral infection of mammalian cells, the compounds are suitable to inhibit and to treat viral infections in mammals, including humans. Viral infectivity inhibition at pharmacologically-acceptable concentrations has been shown in human cell types (see the Examples, below).

Administration of the beta-peptides to a human or non-human patient can be accomplished by any means known. The preferred administration route is parenteral, including intravenous administration, intraarterial administration, intratumor administration, intramuscular administration, intraperitoneal administration, and subcutaneous administration in combination with a pharmaceutical carrier suitable for the chosen administration route. The treatment method is also amenable to oral administration.

As with all pharmaceuticals, the concentration or amount of the beta-peptide administered will vary depending upon the severity of the ailment being treated, the mode of administration, the condition and age of the subject being treated, and the particular .beta.-peptide or combination of beta-peptides being used.

The compounds can be administered in the form of tablets, pills, powder mixtures, capsules, injectables, solutions, suppositories, emulsions, dispersions, food premixes, and in other suitable forms. The pharmaceutical dosage form which contains the compounds described herein is conveniently admixed with a non-toxic pharmaceutical organic carrier or a non-toxic pharmaceutical inorganic carrier.

Typical pharmaceutically-acceptable carriers include, for example, mannitol, urea, dextrans, lactose, potato and maize starches, magnesium stearate, talc, vegetable oils, polyalkylene glycols, ethyl cellulose, poly(vinylpyrrolidone), calcium carbonate, ethyl oleate, isopropyl myristate, benzyl benzoate, sodium carbonate, gelatin, potassium carbonate, silicic acid, and other conventionally employed acceptable carriers. The pharmaceutical dosage form may also contain non-toxic auxiliary substances such as emulsifying, preserving, or wetting agents, and the like.

Solid forms, such as tablets, capsules and powders, can be fabricated using conventional tabletting and capsule-filling machinery, which is well known in the art. Solid dosage forms may contain any number of additional non-active ingredients known to the art, including excipients, lubricants, dessicants, binders, colorants, disintegrating agents, dry flow modifiers, preservatives, and the like.

Liquid forms for ingestion can be formulated using known liquid carriers, including aqueous and non-aqueous carriers, suspensions, oil-in-water and/or water-in-oil emulsions, and the like. Liquid formulation may also contain any number of additional non-active ingredients, including colorants, fragrance, flavorings, viscosity modifiers, preservatives, stabilizers, and the like.

For parenteral administration, the subject compounds may be administered as injectable dosages of a solution or suspension of the compound in a physiologically-acceptable diluent or sterile liquid carrier such as water or oil, with or without additional surfactants or adjuvants. An illustrative list of carrier oils would include animal and vegetable oils (peanut oil, soy bean oil), petroleum-derived oils (mineral oil), and synthetic oils. In general, for injectable unit doses, water, saline, aqueous dextrose and related sugar solutions, and ethanol and glycol solutions such as propylene glycol or polyethylene glycol are preferred liquid carriers.

The pharmaceutical unit dosage chosen is preferably fabricated and administered to provide a concentration of drug at the point of contact with the microbial cell of from about 1 M to 10 mM. More preferred is a concentration of from about 1 to 100 M. As noted earlier, this concentration will, of course, depend on the chosen route of administration and the mass of the subject being treated. Dosage ranges above and below the stated range are within the scope of this invention.

Chemistry:

General. Melting points are uncorrected. CH₂Cl₂ was freshly distilled from CaH₂ under N₂. DMF was distilled under reduced pressure from ninhydrin and stored over 4 angstrom molecular sieves. Triethylamine was distilled from CaH₂ before use. Other solvents and reagents were used as obtained from commercial suppliers. For BOC removal, 4 M HCl in dioxane from was used. Column chromatography was carried out by using low air pressure (typically 6 psi) with 230-400 mesh silica gel 60. Routine ¹³H-NMR spectra were obtained on a Bruker AC-300 and are referenced to residual protonated NMR solvent. Routine ¹³C-NMR spectra were obtained on a Bruker AC-300 and are referenced to the NMR solvent. High resolution electron impact mass spectroscopy was performed on a Kratos MS-80RFA spectrometer with DS55/DS90.

Infrared Spectroscopy. Spectra were obtained on a Nicolet Model 740 FT-IR spectrometer. IR samples were prepared under anhydrous conditions; CH₂Cl₂ was freshly distilled from CaH₂, compounds and glassware were dried under vacuum for 1-2 days, and solutions were prepared under a nitrogen atmosphere. The pure solvent spectrum for a particular solution was subtracted from the sample spectrum prior to analysis. Peaks in the amide NH stretch region were baseline corrected, and analyzed without further manipulation.

NMR Spectroscopy:

1. Aggregation Studies. One-dimensional spectra for aggregation studies were obtained on a Bruker AC-300 spectrometer. Samples for aggregation studies were prepared by serial dilution from the most concentrated sample (50 mM or 27 mM). Dry compounds were dissolved in CD₂Cl₂ previously dried over 3 molecular sieves, and samples were prepared with dry glassware under a nitrogen atmosphere.

2. Conformational Analysis. NMR samples for conformational analysis were prepared by dissolving the dry compound in dry deuterated solvent under a nitrogen atmosphere. CD₂Cl₂ samples were then degassed by the freeze-pump-thaw method, and the NMR tubes were sealed under vacuum. Methanol samples were sealed with a close fitting cap and paraflim. COSY spectra were obtained on a Bruker AC-300 spectrometer. TOCSY (Braunschweiler, L.; Ernst, R. R. (1983) J. Magn. Reson. 53:521), NOESY (Macura, S.; Ernst, R. R. (1980) Mol. Phys. 41:95), and ROESY (Bothner-By, A. A.; Stephens, R. L.; Lee, J.; Warren, C. D.; Jeanloz R. W. (1984) J. Am. Chem. Soc. (1984) 106:811) spectra were squired on a Varian Unity-500 spectrometer using standard Varian pulse sequences and hypercomplex phase cycling (States-Haberkorn method), and the data were processed with Varian “VNMR” version 5.1 software. Proton signals were assigned via COSY and TOCSY spectra, and NOESY and ROESY spectra provided the data used in the conformational analyses. TOCSY spectra were recorded with 2048 points in t₁, 320 or 350 points in t₂, and 8 or 40 scans per t₂ increment. NOESY and ROESY spectra were recorded with a similar number of t₁ and t₂ points, and 32 and 40 scans per t₂ increment, depending on the sample concentration. The width of the spectral window examined was between 2000 and 4000 Hz. Sample concentrations for two-dimensional spectra were 2 mM in CD₂Cl₂ and 8 mM in CD₃OD and CD₃OH.

Far UV Circular Dichroism (CD). Data were obtained on a Jasco J-715 instrument at 20° C. In all CD plots contained herein, the mean residue ellipticity is presented on the vertical axis. Presenting the mean residue ellipticity is a standard practice in peptide chemistry wherein the intensity of each CD spectrum is normalized for the number of amide chromophores in the peptide backbone. Consequently, when the intensities of the maximum (ca. 205 nm) and minimum (ca. 220 um) peaks characteristic of helix formation increase with increasing chain length, this change represents an increase in the population of the helix structure, rather than simply an increase in the number of chromophores present in each molecule.

Synthesis. The beta-amino acids used to assemble the peptides described herein can be manufactured using several different literature methods, as well as the methods described below. For unsubstituted beta-amino acids and beta-amino acids containing one or two acyclic substituents on the carbon adjacent to the amino group in the product beta-peptide, the Arndt-Eisterdt homologation reaction can be used, see Reaction 1. See also Seebach et al. (1996) Helv. Chim. Acta 79:913. This route has advantages and disadvantages. A distinct advantage is that the starting materials, -amino acids, are readily available commercially in enantiomerically pure form. The Arndt-Eisterdt homologation also results in the simultaneous coupling of two beta-amino residues. A distinct disadvantage is that the reaction cannot be used to synthesize beta-amino acids having rings in the backbone or -carbon substituents. The reaction proceeds via a Wolff rearrangement of a diazoketone with subequent trapping of the reactive intermediate with an amino moiety, as shown in Reaction 1:

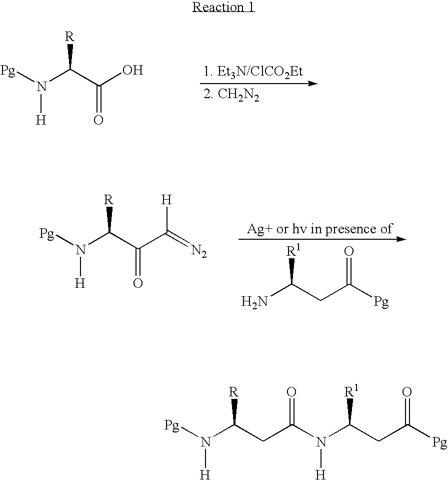

Pg designates any suitable protecting group such as (t-butoxy)carbonyl (Boc) or an adjacent beta-amino residue, R¹ and R² are aliphatic substituents. (Regarding protecting groups, a host of suitable protecting groups for amino moieties, carboxy moieties, and amino acid side-chain moieties are known in the art, and will not be described in any detail herein. For an exhaustive treatment of the subject, see

Beta-Amino acids containing an unsubstituted cycloalkyl moiety involving the and carbons were synthesized using literature methods. See, for example, Nohira et al. (1970) Bull. Chem. Soc. Jpn. 43:2230; Herradon and Seebach (1989) Helv. Chim. Acta 72:690-714; and Tilley et al. (1992) J. Med. Chem. 35:3774-3783, all three of which are incorporated herein by reference.

In particular, the cyclohexyl-containing beta-amino acids can be synthesized via Reaction 2:

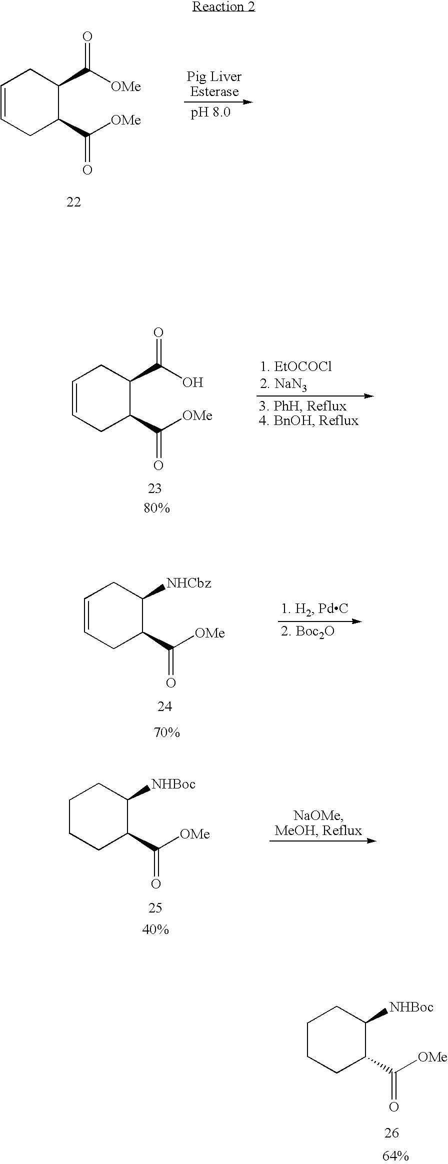

(1R,6S)-6-Methoxycarbonyl-3-cyclohexene-1-carboxylic acid (23): 4600 u of PLE was suspended in pH 8.01 aqueous buffer solution (0.17 M KH₂PO₄). The diester 22 (10.1 g, 0.05 mol) was dissolved in 30 mL of acetone and added to the buffer solution. Reaction was allowed to stir at rt overnight. The enzyme was filtered off through a well-packed celite pad, the solution was then acidified to pH 1 with 1M HCl and the product was extracted with ethyl acetate (5×400 mL). The combined organic extracts were dried over anhydrous magnesium sulfate and concentrated to yield 9.00 g yellow oil. Product taken on without further purification.

Methyl (1S,6R)-6-benzyloxycarbonylaminocyclohex-3-ene carboxylate (24): Ethylchloroforamate (4 mL, 0.042 mol) was added to a mixture of 23 (5.14 g, 0.028 mol) and triethylamine (6 mL, 0.043 mol) in acetone (100 mL) at 0° C. and vigorously stirred for 10 min. An aqueous solution of NaN₃ (3.04 g, 0.047 mol, in 25 mL water) was added in one portion. The resulting mixture was stirred for 30 min at 0° C. The reaction mixture was diluted with water and extracted with diethyl ether. The organic extracts were dried over anhydrous magnesium sulfate and concentrated without heat to yield a viscous yellow liquid. The liquid was dissolved in 100 mL of benzene and refluxed under nitrogen atmosphere for 30 min. Benzyl alcohol (12 mL, 0.116 mol) was added and solution was refluxed for an additional 16 h. The reaction was cooled to rt and concentrated to yield 17.12 g of a yellow liquid (mixture of benzyl alcohol and desired product in a 5.4:1 ratio, respectively by ¹H NMR, 5.67 g product). Mixture taken on without further purification.

Methyl (1S,6R)-6-tert-butoxycarbonylaminocyclohexane carboxylate (25): The yellow oil from the previous reaction, which contains compound 24 (5.6 g, 0.020 mol) and benzyl alcohol, was dissolved in methanol. 0.525 g of 10% Pd on carbon was added to the methanol solution, and the heterogenous mixture was placed under 50 psi H₂ and shaken at rt for 24 h. The mixture was filtered through celite, and the filtrate was concentrated to yield 13.74 g of dark golden yellow liquid. 25 mL of 1M HCl was added to the filtrate, and the benzyl alcohol was extracted with diethyl ether (3×25 mL). The pH of the aqueous solution was adjusted to 9 using K₂CO₃. 25 mL of dioxane and Boc₂O (5 g, 0.023 mol) were added to the solution, and the reaction was stirred at rt for 20 h. 15 mL of water was added and the solution was extracted with ethyl acetate (3×50 mL). The combined organic extracts were dried over anhydrous magnesium sulfate and concentrated. Residue was purified via column chromatography (SiO₂, eluting with 6:1 Hex:EtOAc), to yield 2.00 g viscous clear oil.

Methyl (IR,6R)-6tert-butoxycarbonylaminocyclohexane carboxylate (26): Sodium metal (0.14 g, 6.1 mmol) was placed into a flame dried flask under nitrogen atmosphere and cooled to 0° C. 10 mL of freshly distilled methanol was added and the mixture stirred until all the sodium dissolved. An amount of 25 (2.00 g, 7.7 mmol) was dissolved in 10 mL of freshly distilled methanol and transferred to NaOMe solution via cannula. The solution was refluxed under nitrogen for 5.5 h, cooled to rt and acidified with 0.5 M aqueous 0.5 M ammonium chloride (18 mL, 9 mmol). The methanol was removed under reduced pressure, and the resulting solid collected by filtration to yield 1.27 g of desired product.

Beta-Amino acids containing a substituted cycloalkyl moiety were synthesized using the following illustrative protocol, the first four steps of which are described in Kobayashi et al. (1990) Chem. Pharm. Bull. (1990) 38:350. The remaining steps to yield a cyclohexyl ring having two differentially protected amino substituents were developed in furtherance of the present invention and have not heretofore been described in the literature and are shown in Reaction 3:

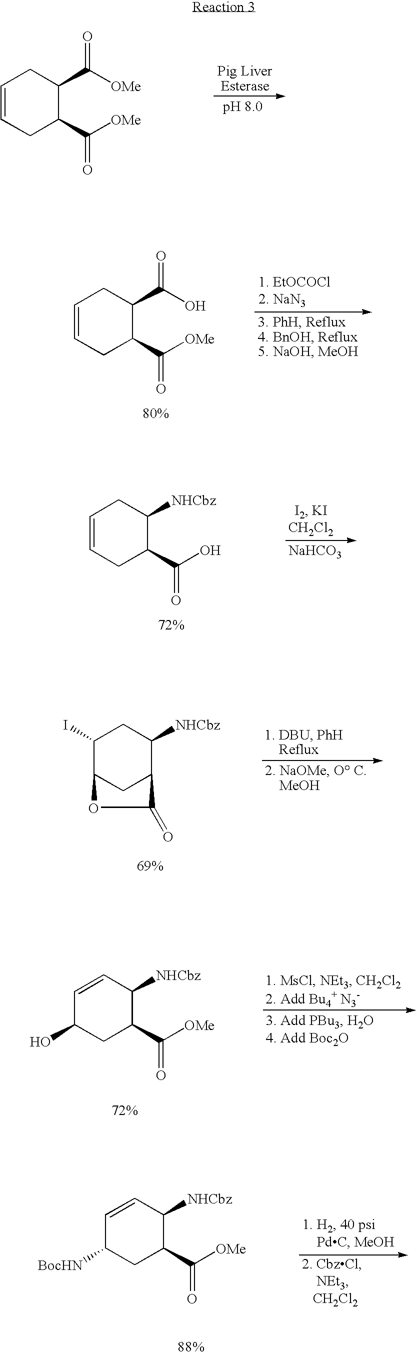

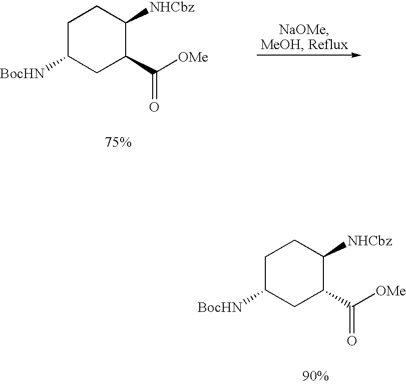

As depicted in Reaction 3, the 4-position amino substituent is protected by a Boc group and the 1-position amino substituent is protected by a Cbz group. The starting material is available commercially (Aldrich Chemical Co., Milwaukee, Wis.).

Synthesis of beta-amino acids containing a heterocylic ring moiety encompassing the alpha- and beta carbons were synthesized using Reactions 4 and 5, below. Reaction 4 details an illustrative synthesis of a beta-proline wherein the exocyclic amino substituent is in the 3-position relative to the ring nitrogen.

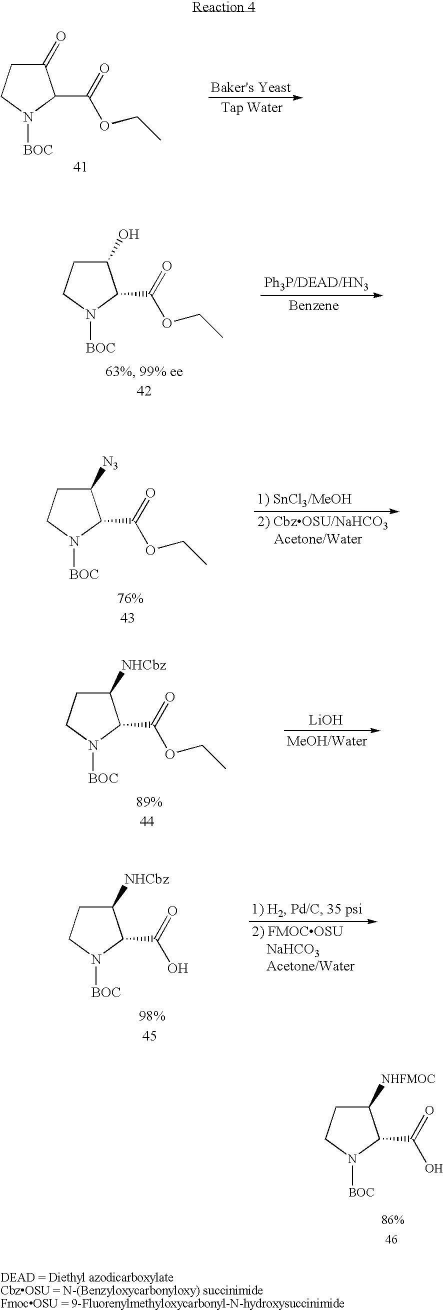

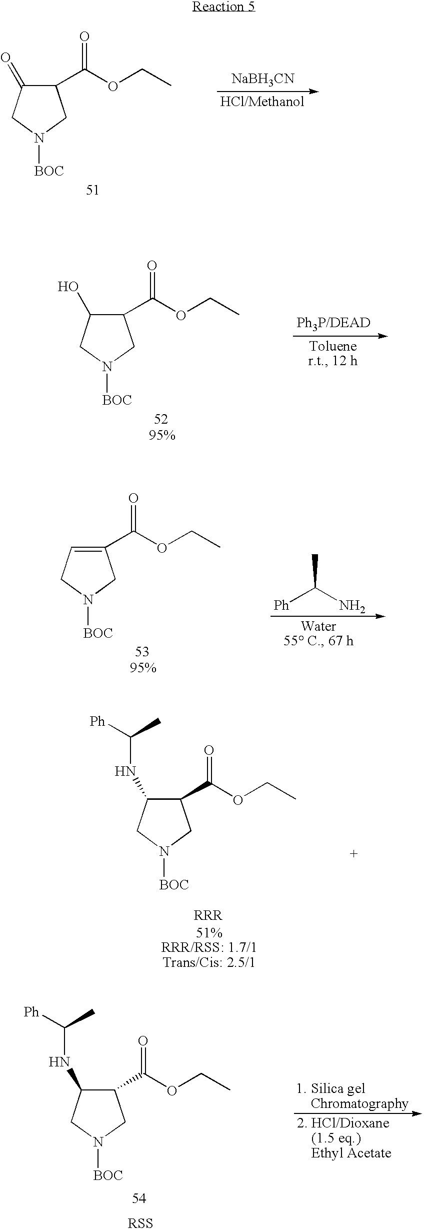

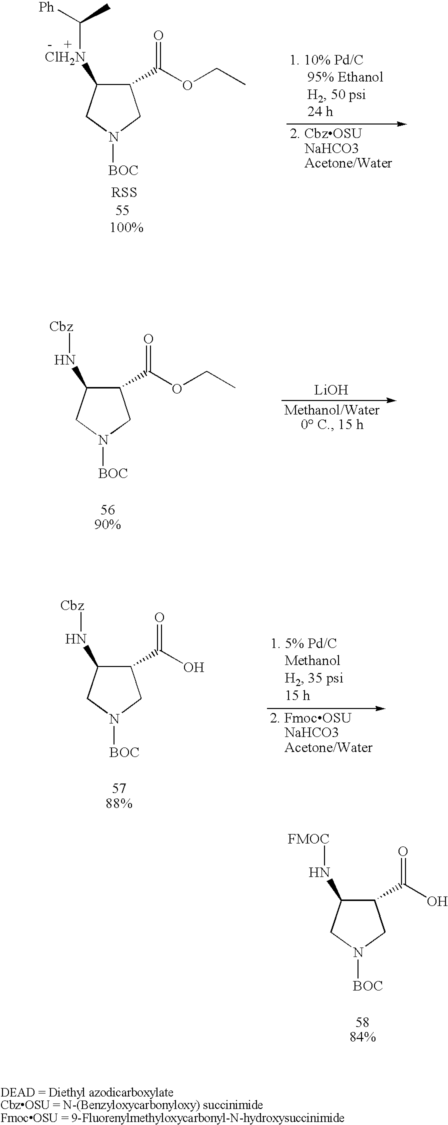

Compound 42: Tap water (200 ml) and baker's yeast (25 g) were mixed, and were shaken on an orbital shaker for 1 hour. Compound 41 (1.0 g) was then added. The mixture was shaken at room temperature for 24 hours. The mixture was filtered through a bed of Celite. The Celite was washed with water (20 ml). The filtrate was extracted with diethyl ether (5×100 ml). The extracts were washed with water (2×50 ml), dried over MgSO₄, and concentrated to yield a slightly yellow oil. The crude product was purified by column chromatography with ethyl acetate/hexane (1/1, v/v) as eluent to give a colorless oil (0.5 g) in 50% yield.

Compound 43: Compound 42 (228 mg) and Ph₃P (346 mg) were dissolved in benzene (anhydrous, 4 ml) under nitrogen. HN₃ (1.64 M in benzene, 0.8 ml) was then added. A solution of diethyl azodicarboxylate (0.18 ml) in benzene (1.0 ml) was subsequently introduced via syringe over 5 minutes. The reaction mixture turned cloudy towards the end of the addition. The reaction mixture was stirred under nitrogen at room temperature for 3.0 hours. The reaction mixture was then taken up in ethyl acetate (50 ml), washed with 1N NaOH (10 ml), saturated NaHCO₃ (10 ml), and finally dilute brine (5 ml). The organic was dried over MgSO₄, and concentrated to give a slightly yellow oil. The crude oil was purified by column chromatography with ethyl acetate/hexane (1/1, v/v) as eluent to afford a colorless oil (190 mg) in 76% yield.

Compound 44: Compound 43 (1.1 g) was dissolved in methanol (50 ml). SnCl₂ (2.2 g) was then added. The mixture was stirred at room temperature for 30 hours. The methanol was then removed under reduced pressure. The residue was dissolved in methylene chloride (50 ml). The resulting cloudy solution was filtered through Celite. The methylene chloride was then removed under reduced pressure. The residual white solid was dissolved in acetone/water (2/1, v/v, 50 ml). NaHCO₃ (3.3 g) was added, followed by Cbz-OSU (1.16 g). The reaction mixture was stirred at room temperature for 24 hours. Water (50 ml) was added. The acetone was removed under reduced pressure. The aqueous mixture was extracted with ethyl acetate (3×100 ml). The extracts were washed with dilute brine (30 ml), dried over MgSO₄, and concentrated to give a colorless oil. The crude product was purified by column chromatography with ethyl acetate/hexane (3/7, v/v) as eluent to give the clean product as a colorless oil (1.35 g) in 89% yield.

Compound 45: Compound 44 (1.35 g) was dissolved in methanol/water (3/1, v/v, 80 ml), cooled to 0° C. LiOH.H2O (1.68 g) was added. The mixture was stirred at 0° C. for 24 hours, by which time TLC indicated that the hydrolysis was complete. Saturated ammonium hydroxide (20 ml) was added. The methanol was removed under reduced pressure. The aqueous was washed with diethyl ether (50 ml), acidified with 1N HCl to pH 3, extracted with methylene chloride (3×150 ml). The extracts were washed with dilute brine (50 ml), dried over MgSO₄, concentrated to give a sticky colorless residue (1.25 g, 99%), which was used directly without further purification.

Compound 46: Compound 45 (1.25 g) was dissolved in methanol (50 ml) in a hydrogenation flask. 5% Palladium on activated carbon (190 mg) was added. The flask was pressurized with hydrogen to 35 psi, rocked at room temperature for 7 hours, by which time TLC indicated that the hydrogenolysis was complete. The Pd/C was removed by filtration. The filtrate was concentrated to give a white solid. The white solid was dissolved in acetone/water (2/1, v/v, 70 ml), cooled to 0° C. NaHCO₃ (1.7 g) was added, followed by FMOC-OSU (1.39 g). The reaction mixture was stirred at room temperature for 16 hours. Water (50 ml) was added. The acetone was removed under reduced pressure. The aqueous was washed with diethyl ether (50 ml), acidified with 1N HCl to pH 3, extracted with methylene chloride (3×150 ml). The extracts were washed with dilute brine (50 ml), dried over MgSO₄, concentrated to give a foamy white solid. The crude white solid was purified by column chromatography with methanol/ethyl acetate (3/7, v/v) as eluent to give the clean product as a white solid (1.3 g) in 86% yield.

Reaction 5 (shown above) illustrates the synthesis of a beta-amino acid wherein the exocyclic amino substituent the nitrogen heteroatom is in the 4-position relative to the ring nitrogen.

Compound 52: Compound 51 (2.0 g) and NaBH₃CN (0.54 g) were dissolved in methanol (40 ml), 1N HCl (aqueous) was added dropwise to maintain pH 3-4. After 15-20 minutes, pH change slowed. The mixture was stirred for an additional 1.0 hour, while 1N HCl was added occasionally to keep pH 3-4. Water (100 ml) was added. The mixture was extracted diethyl ether (3×150 ml). The extracts were washed with IN NaHCO3 (100 ml) and dilute brine (100 ml), dried over MgSO₄, and concentrated to give a colorless oil (1.9 g) in 95% yield. The product was used directly without further purification.

Compound 53: Compound 52 (1.9 g) and Ph₃P (2.8 g) were dissolved in toluene (anhydrous, 30 ml) under nitrogen. A solution of diethyl azodicarboxylate (1.5 ml) in toluene (10 ml) was subsequenely introduced via syringe over 15 minutes. The reaction mixture was stirred under nitrogen at room temperature for 12 hours. The toluene was removed under reduced pressure. The residue was purified by column chromatography with ethyl acetate/hexane (3/7, v/v) as eluent to afford a colorless oil (1.6 g) in 91% yield.

Compound 54: Compound 53 (1.0 g) and R-(+)-methylbenzylamine (1.1 ml) were mixed with water (15 ml). The mixture was stirred at 55° C. for 67 hours. The mixture was taken up in diethyl ether (300 ml), and the aqueous layer was separated. The ether solution was washed with water (3×50 ml), dried over MgSO₄, and concentrated to give a slight yellow oil. The diastereometic isomers were separated by column chromatography with ethyl acetate/hexane (2/8, v/v) as eluent to give RSS (0.2 g) and RRR (0.34 g) in 51% overall yield.

Compound 55: Compound 54 (4.2 g) was dissolved in ethyl acetate (200 ml). 4N HCl in dioxane (4.35 ml) was added dropwise while stirring. A white precipitate resulted. The ethyl acetate was removed under reduced pressure, and the resulting white solid (4.6 g, 100%) was dried in vacuo.

Compound 56: Compound 55 (4.6 g) was dissolved in 95% ethanol (150 ml) in a hydrogenation flask. 10% Palladium on activated carbon (0.5 g) was added. The flask was pressurized with hydrogen to 50 psi, rocked at room temperature for 22 hours, by which time NMR spectroscopy indicated that the hydrogenolysis was complete. The Pd/C was removed by filtration. The filtrate was concentrated to give a white solid. The white solid was dissolved in acetone/water (2/1, v/v, 150 ml). NaHCO₃ (9.7 g) was added, followed by Cbz-OSU (3.4 g). The reaction mixture was stirred at room temperature for 14 hours. Water (100 ml) was added. The acetone was removed under reduced pressure. The aqueous mixture was extracted with ethyl acetate (3×200 ml). The extracts were washed with 1N HCl (3×100 ml) and saturated NaHCO₃ (aqueous), dried over MgSO₄, and concentrated to give a colorless oil. The crude product was purified by column chromatography with ethyl acetate/hexane (3/7, v/v) as eluent lo give the clean product as a colorless sticky oil (4.0 g) in 90% yield.

Compound 57: Compound 56 (2.0 g) was dissolved in methanol/water (3/1, v/v, 115 ml), cooled to 0° C., LiOH.H₂O0 (2.4 g) was added. The mixture was stirred at 0° C. for 15 hours, by which time TLC indicated that the hydrolysis was complete. Saturated ammonium hydroxide (aqueous, 100 ml) was added. The methanol was removed under reduced pressure. The aqueous was acidified with 1N HCl to pH 3, extracted with ethyl acetate (3×200 ml). The extracts were washed with dilute brine (100 ml), dried over MgSO₄, concentrated to give a foamy solid (1.63 g, 88%), which was used directly without further purification).

Compound 58: Compound 57 (1.63 g) was dissolved in methanol (70 ml) in a hydrogenation flask. 5% Palladium on activated carbon (250 mg) was added. The flask was pressurized with hydrogen to 35 psi, rocked at room temperature for 15 hours, by which time NMR spectroscopy indicated that the hydrogenolysis was complete. The Pd/C was removed by filtration. The filtrate was concentrated to ive a white solid. The white solid was dissolved in acetone/water (2/1, v/v, 90 ml), cooled to 0° C. NaHCO₃ (2.27 g) was added, followed by FMOC-OSU (1.83 g). The reaction mixture was stirred at 0° C. for 2 hours, then at room temperature for 28 hours. Water (50 ml) was added. The acetone was removed under reduced pressure. The aqueous was acidified with 1N HCl to pH 3, extracted with ethyl acetate (3×200 ml). The extracts were washed with dilute brine (100 ml), dried over MgSO₄, concentrated to give a foamy white solid. The crude white solid was purified by column chromatography with methanolfethyl acetate (3/7, v/v) as eluent to give the clean product as a white solid (1.68 g) in 84% yield.

To synthesize the nipecotic reverse turn moiety, Reaction 6 was used.

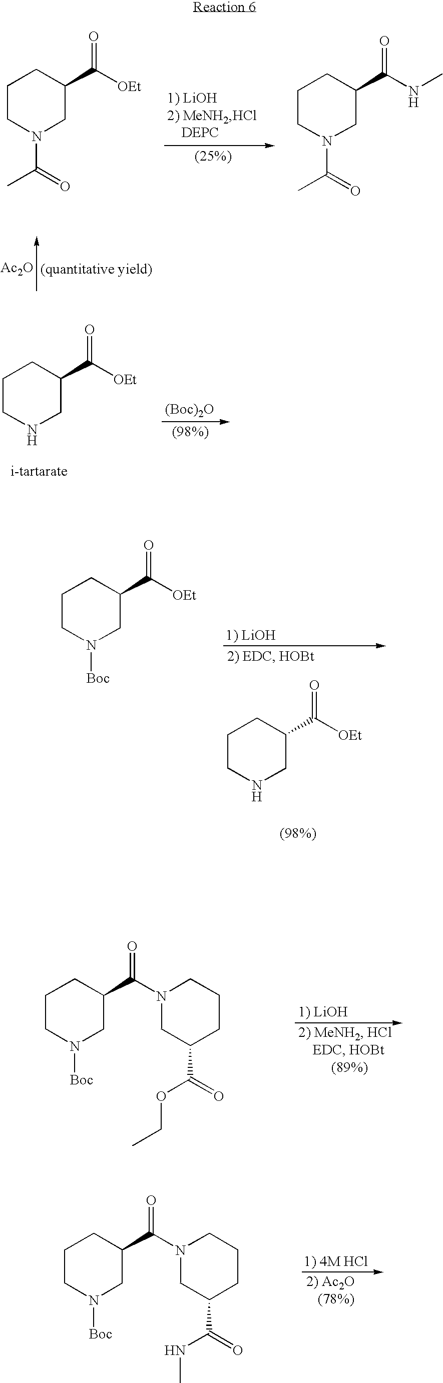

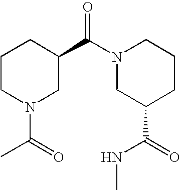

To synthesize beta-peptides having reverse turn moiety which is a prolyl glycolic acid residue, the following protocols are preferred:

(2S,3R)-3-Amino-2-methylpentanoic acid was prepared according to the procedures given by Jefford and McNulty (1994), J. Helv. Chim. Acta 77:2142. However, unlike the description in this paper, the synthesized (2S,3S)-2-methyl-3-(tosylamino)butano-4-lactone contained up to 8% (2R,3S)-2-methyl-3-(tosylamino)butano-4-lactone as a byproduct, which could be removed by recrystallization from toluene. (2S,3S)-3-Amino-2-benzyl-4-phenylthiobutanoic acid was prepared in a synthetic sequence derived from the one by Jefford and McNulty. This synthesis is described below. Homo-beta-amino acids were prepared according to the procedures by Podlech and Seebach (1995), Liebigs Ann. 1217. Depsi-beta-peptides were synthesized by conventional dicyclohexylcarbodiimide/N-hydroxysuccinimide (DCC/HOSu) or 1-ethyl-3-(3′-dimethylanrinopropyl)carbodiimidde hydrochloride/N,N-dimethyl-4-aminopyridine (EDCI/DMAP) solution-phase coupling procedures (see, for example, Bodanszky, M.; Bodanszky, A. The Practice of Peptide Synthesis; Springer Verlag: New York, 1984). Illustrative procedures are given below.

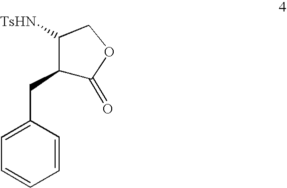

(2S,3S)-2-Benzyl-3-(tosylamino)butano-4-lactone (4). A solution of lithium diisopropylamine (LDA) in THF was generated by adding 1.5 M methyllithium in diethyl ether (30 mL, 45.0 mmol) to a solution of diisopropylamine (6.4 mL, 45.7 mmol) in 100 mL THF at 0° C. under nitrogen and stirring for 10 min. The solution was then cooled to −78° C., and a solution of (3S)-3-(tosylamino)butano-4-lactone (5.36 g, 21.1 mmol) in 30 mL THF was added dropwise. The resulting yellow solution was stirred for 1 hour at −78° C., and then benzyl bromide (10 mL, 84.1 mmol) was added rapidly. Stirring at −78° C. was continued for 2 hours, and the reaction was quenched with 20 mL sat. aq. NH₄Cl solution and allowed to warm to room temperature. The mixture was acidified with 1 M HCl and extracted three times with methylene chloride. The combined organic extracts were dried over Na₂SO₄ and evaporated to give an orange semisolid that was purified by chromatography (silica gel, hexane/ethyl acetate 3:2) to yield 2.22 g (8.70 mmol, 41%) recovered starting material and 3.37 g (9.76 mmol; 46%) of 4. No diastereomeric addition product could be detected. For further purification 4 can be recrystallized from toluene to give colorless needles. mp. 108.5-109° C., ¹H-NMR (300 MHz, CDCl₃) 7.58 (d, J=8.5 Hz, 2H), 7.31 (d, J=8.2 Hz, 2H), 7.20 (m, 3H), 6.92 (dd, J=7.7, 1.7 Hz, 2H), 4.97 (d, J=5.5 Hz, 1H), 4.27 (dd, J=7.2, 9.8 Hz, 1H), 3.98 (dd, J=7.2, 9.8 Hz, 1H), 3.65 (m, 1H), 3.00 (m, 1 H), 2.77 (m, 2H), 2.46 (s, 3H), ¹³C-NMR (75.5 MHz, CDCl₃) 144.27 (C), 138.00 (C), 135.83 (C), 129.89 (CH), 128.97 (CH), 128.88 (CH), 127.10 (CH) 71.25 (CH₂) 53.21 (CH), 46.54 (CH), 33.54 (CH₂) 21.50 (CH₃), EI MS m/e 345.1027 calc. for C₁₈H,₁₉NO₄S 345.1035.

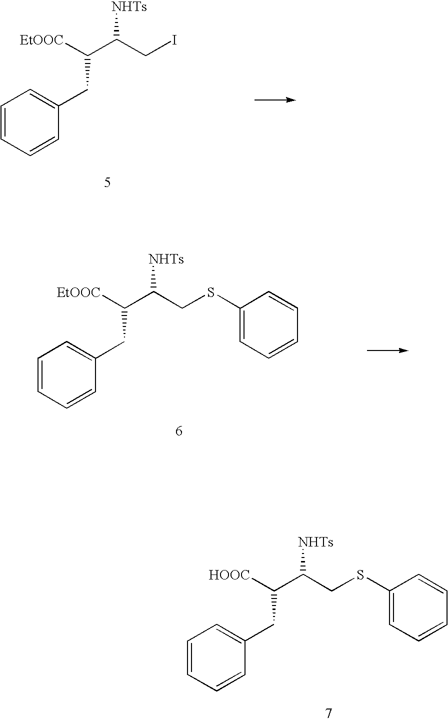

(2S,3S)-2-Benzyl-4-phenylthio-3-(tosylamino)butanoic acid (7). (2S,3S)-2-Benzyl-3-(tosylamino)butano-4-lactone (4) (0.91 g, 2.64 mmol) was dissolved in 10 mL methylene chloride. At 0° C. trimethylsilyliodide (1 mL, 7.03 mmol) and anhydrous ethanol (0.72 mL, 12.2 mmol) were added under nitrogen. The solution was stirred 30 min. at 0° C., allowed to warm to room temperature and stirred for 1 day. Then the addition of trimethylsilyliodide and ethanol was repeated and stirring at room temperature was continued for 12 hours. The reaction was quenched by the addition of 3 mL ethanol and stirring for 30 min. To the solution 20 mL of water were added, the layers were separated, and the aqueous layer was extracted five times with methylene chloride. The combined organic extracts were washed with 5% aq. Na₂S₂O₃ solution, dried over Na₂SO₄ and concentrated in vacuo to give 1.78 g of crude 5 as an orange solid, which was used in the next step without further purification.

At 0° C., thiophenol (0.73 ml, 7.11 mmol) was added to a suspension of NaH (289.7 mg, 7.24 mmol) in 6 mL DMF under nitrogen, warmed to room temperature and stirred for 15 min. A solution of crude 5 (1.78 g) in 10 mL DMF was added to the thiophenolate solution at 0° C. After warming to room temperature the solution was stirred for 1 hour. The reaction was quenched with 50 ml water and extracted three times with methylene chloride. The combined organic extracts were washed with brine, dried over Na₂SO₄ and concentrated in vacuo to give 2.43 g of 6 as a colorless oil, which was used in the next step without further purification.

To a solution of 6 (2.43 g) in 18 mL methanol a 1.5 M aq. NaOH solution was added and the mixture heated to 60° C. for 2 hours. After evaporation of methanol in vacuo, 20 mL water was added and the mixture extracted two times with diethyl ether. The aqueous layer was acidified with conc. HCl and extracted four times with diethyl ether. The organic extracts were dried over Na₂SO₄ and evaporated to yield 1.04 g (2.28 mmol, 86%) of 7. ¹H-NMR (300 MHz, CDCl₃) 7.45 (d, J=8.3 Hz, 2H), 7.24-7.17 (m, 6H), 7.09-7.00 (m, 6H), 5.54 (d, J=8.3 Hz, NH), 3.46 (m, 1H), 3.28 (m, 1H), 3.00 (m, 3H), 2.67 (dd, J=7.1, 14.0 Hz, 1H), 2.34 (s, 3H).

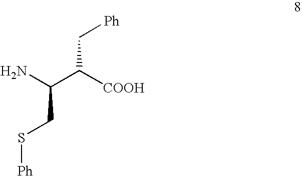

(2S,3S)-3-Amino-2-Benzyl-4-phenylthiobutanoic acid (8). Compound 7 and phenol (0.77 g) were dissolved in 50 mL 48% HBr and heated to reflux for 1.5 hours under nitrogen. After cooling to room temperature 150 mL water was added and the solution extracted two times with diethyl ether. The yellow aqueous layer was evaporated to give 0.58 g of (2S,3S)-3-amino-2-benzyl-4-phenylthiobutanoic acid hydrobromide as an orange solid. ¹H-NMR (300 MHz, CDCl₃) 7.69 (b, 3 NH), 7.43 (m, 2H), 7.34-7.01 (m 8H), 3.60 (m, 1H), 3.35 (m, 3H), 3.08 (dd, J=8.2, 14.2 Hz, 1H), 2.87 (dd, J=7.5, 14.2 Hz, 1H).

The hydrobromide was dissolved in 140 mL anhydrous ethanol, and 28 mL methyloxirane was added. The solution was heated to reflux for 1 hour under nitrogen. The solvent was evaporated to yield 0.45 g (1.45 mmol, 65%) of 8.

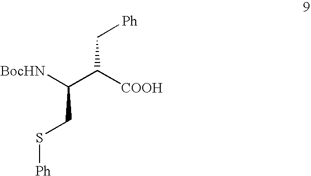

(2S,3S)-3-(t-Butoxycarbonylamino)-2-benzyl-4-phenylthiobutanoic acid. To a solution of 8 (0.18 g, 0.597 mmol) in 1 mL water and 2 mL dioxane was added K₂CO₃ (167.9 mg, 1.21 mmol). After cooling to 0° C., di-t-butyl-dicarbonate (153.2 mg, 0.681 mmol) was added, the solution warmed to room temperature and stirred for 1 day. The solution was concentrated in vacuo, and the residue dissolved in 20 mL water. The solution was acidified to pH 2-3 (congo red) with 1 M HCl and extracted five times with ethyl acetate. The combined organic extracts were dried over MgSO₄ and evaporated to give an orange oil that was purified by chromatography (silica gel, hexane/ethyl acetate 1:2) to yield 63.4 mg (0.159 mmol, 27%) of 9. ¹H-NMR (300 MHz, CDCl₃) 7.37-7.13 (m, 10H), 5.47 (d, J=8.5 Hz, NH), 3.88 (m, 1H), 3.20 (m, 1H), 3.00 (m, 1H), 2.84 (m, 3H), 1.39 (s, 9H), ¹³C-NMR (75.5 MHz, CDCl₃) 174.82 (C), 156.49 (C), 140.14 (C), 136.80 (C), 130.44 (C), 130.02 (C), 129.81 (C), 129.36 (C), 127.33 (C), 127.27 (C), 79.68 (C), 52.46 (CH), 52.33 (CH), 37.37 (CH₂), 35.25 (CH₂), 28.55 (3 CH₃).

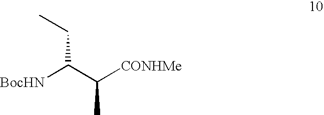

Methyl-(2S,3R)-3-(t-butoxycarbonylamino)-2-methylpentanoic amide (10). (2S,3R)-3-(t-Butoxycarbonylamino)-2-methylpentanoic acid (149.1 mg, 0.645 mmol) was dissolved in 1 mL DMF. At 0° C. Methylamine hydrochloride (88.6 mg, 1.31 mmol) and DMAP (195.7 mg, 1.60 mmol) were added, followed by EDCI (1-ethyl-3-(3′dimethylaminopropyl)carbodiimidde hydrochloride) (376.9 mg, 1.97 mmol). After stirring at room temperature for 2 days, the solvent was removed in a stream of nitrogen and the residue dried in vacuo. The residue was titurated with 1 mL 1 M HCl and 4 mL water, and the white precipitate was collected by suction filtration to yield 121.0 mg (0.495 mmol, 66%) of the amide 10 mp. 206-207° C., ¹H-NMR (300 MHz, CDCl₃) 5.92 (b, NH), 4.72 (b, NH), 3.58 (m, 1H), 2.77 (d, J=4.8 Hz, 3H), 2.45 (m, 1H), 1.45 (m, 1H), 1.41 (s, 9H), 1.40 (m, 1H), 1.13 (d, J=7.2 Hz, 3H), 0.90 (t, J=7.4 Hz, 3H), ¹³C-NMR (75.5 MHz, CDCl₃) 174.83 (C), 156.15 (C), 79.35 (C), 54.62 (CH), 45.02 (CH), 28.35 (3 CH₃), 26.24 (CH₃), 25.18 (CH₂), 13.71 (CH₃), 10.85 (CH₃), EI MS m/e 244.1789 calc. for C₁₂H₂₄N₂O₃ 244.1787.

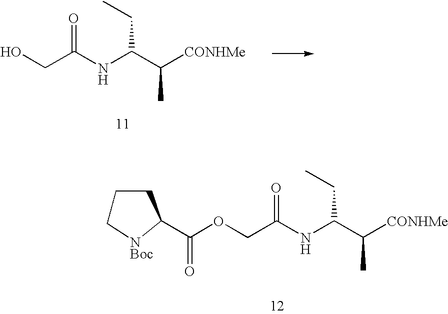

Compound 12. Compound 10 (121.0 mg, 0.495 mmol) was dissolved in 2 mL of 4 M HCl/dioxane, and the resulting solution was stirred 1 hour at room temperature. HCl/dioxane was then removed in a stream of nitrogen and the deprotected amide dried in vacuo. The activated glycolic ester was prepared by adding EDCI (188.7 mg, 0.635 mmol) to a solution of glycolic acid (45.5 mg, 0.598 mmol) and HOSu (N-hydroxysuccinimide) (72.7 mg, 0.632 mmol) in 1 mL DMF and stirring of the solution at room temperature for 2 hours. The deprotected amide and triethylamine (85 μl, 0.610 mmol) were dissolved in 1 mL DMF and transferred into the activated ester solution. After stirring the resulting solution for 2 days at room temperature, the solvent was removed in a stream of nitrogen and the residue dried in vacuo. The residue was separated by chromatography (silica gel, CHCl₃/MeOH 4:1) to yield impure 11 (192.7 mg), which was used in the next step without further purification.

Compound 11 (192.7 mg) and BOC-L-proline (213.3 mg, 0.991 mmol) were dissolved in 3 mL DMF. DMAP (15.6 mg, 0.128 mmol) was added, followed by DCC (dicyclohexylcarbodiimide) (248.3 mg, 1.20 mmol), and the resulting solution was stirred overnight at room temperature. The white precipitate was filtered off by suction filtration, and the filtrate was concentrated in vacuo. The residue was separated by chromatography (silica gel, CHCl₃/MeOH 19:1) to yield 145.7 mg (0.365 mmol, 74% based on 10) of 12. ¹H-NMR (300 MHz, CDCl₃) 7.02 (d, J=8.6 Hz, NH major rotamer 89%), 6.91 (d, J=9.0 Hz, NH minor rotamer 11%), 6.10 (m, NH), 4.78 (AB, A part, J=15.3 Hz, 1H), 4.49 (AB, B part, J=15.3 Hz, 1H) 4.26 (m, 1H), 3.90 (m, 1H), 3.44 (m, 2H), 2.74 (d, J=4.6 Hz, 3H), 2.45 (quint., J=7.0 Hz, 1H), 2.22 (m, 1H), 1.98 (m, 2H), 1.88 (m, 1H), 1.56 (m, 1H), 1.43 (s, 9H), 1.43 (m, 1H), 1.08 (d, J=7.0 Hz, 3H), 0.86 (t, J=7.4 Hz), ¹³C-NMR (75.5 MHz, CDCl₃) 174.80 (C), 172.24 (C), 167.47 (C), 154.78 (C), 80.33 (C), 62.79 (CH₂), 58.77 (CH), 53.74 (CH), 46.75 (CH₂), 45.58 (CH), 29.91 (CH₂), 28.26 (3 CH₃), 26.05 (CH₃), 24.97 (CH₂), 24.49 (CH₂), 14.43 (CH), 10.62 (CH₃).

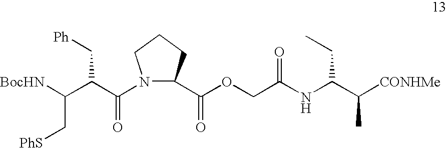

Compound 13. Compound 12 (12.3 mg, 30.8 μmol) was dissolved in 1 mmol 4 M HCl/dioxane and the solution was stirred for 1 hour at room temperature. HCl/dioxane was removed in a stream of nitrogen and the residue dried in vacuo. The deprotected depsipeptide and (2S,3S)-2-benzyl-3-(t-butoxycarbonylamino)-4-phenylthiobutanoic acid (9) (14.3 mg, 35.6 μmol) were dissolved in 0.5 mL methylene chloride. DMAP (5.0 mg, 40.9 μmol) was added, followed by EDCI (13.7 mg, 71.5 μmol). After stirring at room temperature for 2 days, the solvent was removed in a stream of nitrogen and the residue dried in vacuo. The residue was titurated with 1 mL water, which was acidified to pH 2. The resulting solid was collected and purified by chromatography (silica gel, CHC1₃/MeOH 19:1) to yield 14.1 mg (20.6 μmol, 67%) of 13. ¹H-NMR (300 MHz, CDCl₃) 7.41 (d, J=10.1 Hz, NH), 7.38-7.13 (m, 10 H+NH), 5.06 (AB, A part, J=15.3 Hz, 1H), 5.03 (d, J=10.5 Hz, NH), 4.45 (m, 1H), 4.32 (AB, B part, J=15.5 Hz, 1H), 4.26 (m, 1H), 4.02 (t, J=7.6 Hz, 1H), 3.31 (m, 1H), 3.08 (m, 3H), 2.97 (m, 1H) 2.83 (m, 1H), 2.78 (d, J=4.6 Hz, 3H), 2.53 (m, 1H), 2.39 (dq, J=10.1 Hz, 6.9 Hz, 1H), 1.98 (m, 1H), 1.73 (m, 2H), 1.50 (m, 1H), 1.44 (m, 1H), 1.41 (s, 9H), 1.30 (m, 1H), 1.07 (d, J=6.9 Hz, 3H), 0.98 (t, J=7.4 Hz, 3H), ¹³C-NMR (75.5 MHz, CDCl₃) 175.44 (C), 171.63 (C), 167.03 (C), 155.54 (C), 138.29 (C), 135,46 (C), 129.43 (CH), 129.06 (CH), 128.68 (CH), 128.30 (CH), 126.60 (CH), 126.47 (CH), 80.24 (C), 62.69 (CH₂), 59.27 (CH), 52.71 (CH), 52.46 (CH), 49.39 (CH), 46.75 (CH₂), 46.66 (CH), 38.16 (CH₂), 36.32 (CH₂), 28.58 (CH₂), 28.11 (3 CH₃), 26.26 (CH₂), 25.85 (CH₃), 25.05 (CH₂), 16.2 (CH₃), 10.47 (CH₃).

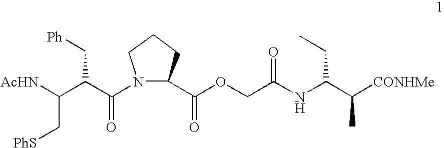

Compound 1. Compound 13 (14.1 mg, 20.6 μmol) was dissolved in 1 mL 4 M HCl/dioxane and the solution was stirred for 1 hour at room temperature. HCl/dioxane was removed in a stream of nitrogen and the residue dried in vacuo. The deprotected depsipeptide and triethylamine (5.8 μL, 41.6 μmol) were dissolved in 0.41 mL methylene chloride, and acetic anhydride (2.4 μL, 25.4 μmol) was added. After stirring the solution at room temperature overnight the solvent was removed in a stream of nitrogen and the residue dried in vacuo. The residue was purified by chromatography (silica gel, CHCl₃/MeOH 19:1) to yield 9.2 mg (14.7 μmol, 71%) of 1. mp. 196.5-197° C., ¹H-NMR (300 MHz, CDCl₃) 7.40 (d, J=9.0 Hz, NH), 7.39-7.11 (m, 10 H+NH), 5.99 (d, J=10.1 Hz, NH), 5.03 (AB, A part, J=15.3 Hz, 1H), 4.78 (tt, J=10.3 Hz, 3.6 Hz, 1H), 4.34 (AB, B part, J =15.3 Hz, 1H), 4.25 (dq, J=10.0 Hz, 1H), 4.02 (t, J=7.4 Hz, 1H), 3.36 (m, 1H), 3.20-3.00 (m, 3H), 2.85-2.75 (m, 2H), 2.79 (d, J=4.6 Hz, 3H), 2.60 (m, 1H), 2.42 (dq, J=10.1 Hz, 6.9 Hz, 1H), 2.00 (m, 1H), 1.86 (s, 3H), 1.85-1.62 (m, 3H), 1.52 (m, 1H), 1.31 (m, 1H), 1.07 (d, J=6.8 Hz, 3H), 0.97 (t, J=7.4 Hz, 3H), ¹³C-NMR (75.5 MHz, CDCl₃) 175.46 (C), 171.59 (C), 170.17 (C), 167.08 (C), 138.14 (C), 136.87 (C), 135,36 (C), 129.49 (CH), 129.15 (CH), 128.60 (CH), 128.34 (CH), 126.88 (CH), 126.64 (CH), 62.69 (CH₂), 59.30 (CH), 52.80 (CH), 51.08 (CH), 48.69 (CH), 46.83 (CH₂), 46.28 (CH), 37.37 (CH₂), 36.30 (CH₂), 34.45 (CH₂), 28.59 (CH₂), 26.03 (CH₃), 25.07 (CH₂), 23.00 (CH₃) 16.09 (CH₃), 10.46 (CH₃), IR (1 mM in CH₂Cl₂) 3423, 3367, 1753, 1669, 1626 cm⁻¹, EI MS m/e 624.2989 calc. for C₃₃H₄₄N₄O₆S 624.2981.

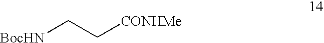

Methyl-3-(t-butoxycarbonylamino)propionic amide (14). BOC7-alanine (0.50 g, 2.64 mmol) was dissolved in 4 mL DMF. Methylamine hydrochloride (198 mg, 2.93 mmol) and DMAP (427.2 mg, 3.50 mmol) were added, followed by EDCI (1.06 g, 5.53 mmol). After stirring at room temperature for 2 days the solvent was removed in a stream of nitrogen and the residue dried in vacuo. It was dissolved in 5 mL 1 M HCl, and the solution was extracted five times with ethyl acetate. The combined organic extracts were dried over MgSO₄ and concentrated to yield 0.43 g (2.13 mmol, 81%) of BOC-alanine methylamide (14) as a white solid. mp. 117-118° C., ¹H-NMR (300 MHz, CDCl₃) 5.78 (b, NH), 5.15 (b, NH), 3.38 (q, J=6.1 Hz, 2H), 2.78 (d, J=4.8 Hz, 3H), 2.36 (t, J=6.1 Hz, 2H), 1.40 (s, 9H) ¹³C—NMR (75.5 MHz, CDC1₃) 171.74 (C), 79.15 (C), 36.41 (CH₂), 36.03 (CH₂), 28.17 (3 CH₃), 26.04 (CH₃).

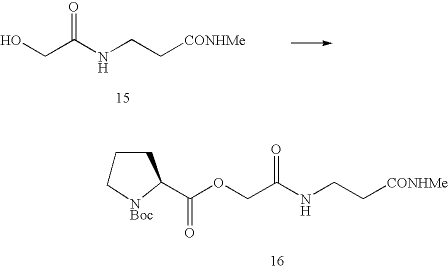

Compound 16. Compound 14 (0.33 g, 1.63 mmol) was dissolved in 5 mL of 4 M HCl/dioxane, and the solution was stirred at 12° C. for 1 hour. The HCl/dioxane was removed in a stream of nitrogen and the residue dried in vacuo. An activated ester solution was prepared by adding DCC (509.9 mg, 2.47 mmol) to a solution of glycolic acid (145.7 mg, 1.92 mmol) and HOSu (326.4 mg, 2.84 mmol) in 10 mL methylene chloride. A white precipitate formed after a few minutes. The suspension was stirred at 12° C. for 6 hours. The deprotected amide and triethylamine (0.27 mL, 1.94 mmol) were dissolved in 10 mL methylene chloride and transferred into the activated ester solution. After stirring the resulting solution overnight at room temperature, the white precipitate was filtered off by suction filtration and the filtrate concentrated to give a white solid, which was purified by chromatography (silica gel, CHCl₃/MEOH 19:1) to yield 0.30 g of impure 15, which was used in the next step without further purification.

Compound 15 (0.30 g) and BOC-L-proline (371.5 mg, 1.73 mmol) were dissolved in 50 mL methylene chloride. At 0° C. DMAP (25.6 mg, 0.210 mmol) was added, followed by DCC (402.9 mg, 1.95 mmol). After stirring 1 hour at 0° C. the suspension was allowed to warm to room temperature and stirred overnight. The white precipitate was filtered off by suction filtration and the filtrate concentrated. The residue was subjected to chromatography (silica gel, CHCl₃/MEOH 19:1) to yield 0.23 g (0.644 mmol, 40% based on 14) of 15 as a colorless glass. ¹H-NMR (300 MHz, CDCl₃) 7.55 (b, NH major rotamer 84%), 7.05 (b, NH minor rotamer 16%), 6.25 (b, NH major rotamer 83%), 6.04 (b, NH, minor rotamer 17%), 4.59 (s, 2H), 4.25 (m, 1H), 3.59 (m, 1H), 3.42 (m, 3H), 2.73 (d, J=4.8 Hz, 3H), 2.41 (t, J=6.5 Hz, 2H), 2.22 (m, 1H), 1.96 (m, 2H), 1.88 (m, 1H), 1.42 (s, 9 H).

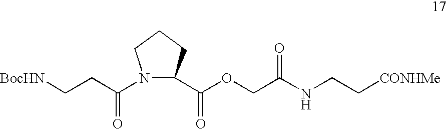

Compound 17. Compound 16 (0.23 g, 0.644 mmol) was dissolved in 2 mL 4 M HCl/dioxane, and the solution was stirred for 1 hour at room temperature. HCl/dioxane was removed in a stream of nitrogen and the residue dried in vacuo. The deprotected depsipeptide and BOC-alanine (133.3 mg, 0.705 mmol) were dissolved in 5 mL methylene chloride. DMAP (96.9 mg, 0.793 mmol) was added, followed by EDCI (258.7 mg, 1.349 mmol). After stirring at room temperature for 2 days the solvent was removed in a stream of nitrogen. The residue was dissolved in 0.1 M HCl and the solution was extracted four times with methylene chloride. The combined organic extracts were dried over MgSO₄ and concentrated to give a white solid that was purified by chromatography (silica gel, CHCl₃/MeOH 19:1) to yield 0.18 g (0.420 mmol, 66%) of 17 as a white solid. ¹H-NMR (300 MHz, CDCl₃) 7.54 (b, NH), 6.30 (b, NH), 5.58 (b, NH), 4.66 (AB, A part, J=15.4 Hz, 1H), 4.47 (AB, B part, J=15.5 Hz, 1H), 4.35 (m, 1H), 3.51 (m, 4H), 3.35 (m, 2H), 2.72 (d, J=4.8 Hz, 3H), 2.51 (m, 2H), 2.41 (m, 2H), 2.20 (m, 1H), 2.10 (m, 1H), 1.98 (m, 2H), 1.37 (s, 9H).

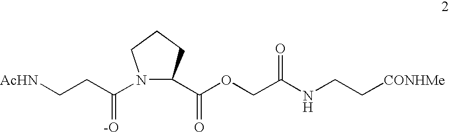

Compound 2. Compound 17 (0.18 g, 0.420 mmol) was dissolved in 2 mL 4 M HCl/dioxane and the solution was stirred for 1 hour at room temperature. HCl/dioxane was removed in a stream of nitrogen and the residue dried in vacuo. The deprotected depsipeptide and triethylamine (0.12 mL, 0.861 mmol) were dissolved in 5 mL methylene chloride. At 0° C. acetic anhydride (50 μL, 0.530 mmol) was added and the solution was stirred 1 hour at 0° C. and then allowed to warm to room temperature with stirring overnight. The solvent was removed in a stream of nitrogen and the residue dried in vacuo. The remaining white solid was purified by chromatography (silica gel, CHCl₃/MeOH 19:1) to yield 0.12 g (0.324 mmol, 77%) of 2 as a white solid. mp. 153.5-154° C., ¹H-NMR (300 MHz, CDCl₃) 7.79 (d, J=4.4 Hz, NH), 7.32 (d, J=3.9 Hz, NH), 6.08 (b, NH), 4.75 (AB, A part, J 15.4 Hz, 1H), 4.44 (AB, B part, J=15.3 Hz, 1H), 4.32 (m, 1H), 3.62-3.40 (m, 5H), 2.74 (d, J=4.8 Hz, 3H), 2.59-2.34 (m, 4H), 2.25-1.91 (m, 3H), 1.97 (s, 3H), ¹³C-NMR (75.5 MHz, CDCl₃) 171.57 (C), 171.30 (C), 170.63 (C), 167.42 (C), 62.78 (CH₂), 59.10 (CH), 47.02 (CH₂), 35.83 (CH₂), 35.54 (CH₂), 34.69 (CH₂), 33.75 (CH₂), 29.00 (CH₂), 26.17 (CH₃), 25.05 (CH₂), 22.83 (CH₃) IR (1 mM in CH₂Cl₂) 3452, 3334, 1757, 1669, 1635, 1539 cm⁻¹, EI MS m/e 370.1868 calc. for C₁₆H₂₆N₄O₆ 370.1852.

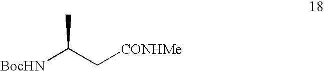

Methyl-(S)-3-(t-butoxycarbonylamino)butanoic amide (18). BOC-homoalanine (Podlech, J.; Seebach, D. (1995) Liebigs Ann. 1217) (0.44 g, 2.17 mmol) was dissolved in 5 mL methylene chloride. HOSu (376.4 mg, 3.27 mmol) was added and the solution cooled to 0° C. After addition of DCC (587.8 mg, 2.85 mmol) the solution was stirred 1 hour at 0° C., warmed to room temperature and stirred for an additional 2 hours. A stream of methylamine was bubbled through the suspension for 10 minutes, and stirring was continued overnight. The white precipitate was filtered off by suction filtration and the filtrate concentrated to give a pale yellow solid that was purified by chromatography (silica gel, CHCl₃/MeOH 19:1) to yield 0.41 g (1.90 mmol, 88%) Of BOC-homoalanine methylamide (18) as a white solid. ¹H-NMR (300 MHz, CDCl₃) 6.11 (b, NH), 5.23 (b, NH), 3.92 (m, 1H), 2.75 (d, J=4.8 Hz, 3H), 2.35 (m, 2H), 1.39 (s, 9H), 1.17 (d, J=6.6 Hz, 3H).

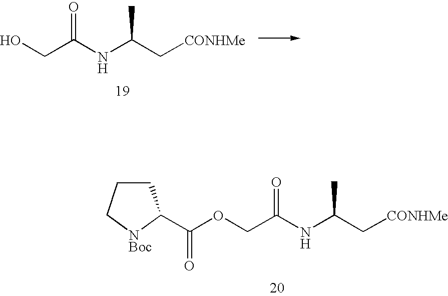

Compound 20. Compound 18 (0.41 g, 1.90 mmol) was dissolved in 2 mL of 4 M HCl/dioxane, and the solution was stirred at room temperature for 1 hour. HCl/dioxane was removed in a stream of nitrogen and the residue dried in vacuo. An activated ester solution was prepared by adding DCC (0.59 g, 2.86 mmol) to a solution of glycolic acid (175.5 mg, 2.31 mmol) and HOSu (421.9 mg, 3.67 mmol) in 5 mL DMF at 0° C. The suspension was stirred at 0° C. for 1 hour and then 2 hours at room temperature. The deprotected amide and triethylamine (0.32 mL, 2.30 mmol) were dissolved in 5 mL DMF and transferred into the activated ester solution. After stirring the resulting solution overnight at room temperature the white precipitate was filtered off by suction filtration and the filtrate concentrated to give a semisolid that was chromatographed (silica gel, CHCl₃/MeOH 9: 1) to yield 0.42 g of impure 19, which was used in the next step without further purification.

Compound 19 (55 mg, 0.317 mmol, impure) and BOC-D-proline (148 mg, 0.688 mmol) were dissolved in 2 mL DMF. DMAP (10.0 mg, 0.082 mmol) was added, followed by DCC (171.4 mg, 0.831 mmol). After stirring the resulting suspension for 1 day at room temperature the white precipitate was filtered off by suction filtration and the filtrate concentrated. The remaining semisolid was purified by chromatography (silica gel, CHCl3/MeOH 19:1) to yield 52.1 mg (0.140 mmol, 44%) of 20. ¹H-NMR (300 MHz, CDC1₃) 7.53 (d, J=6.3 Hz, NH minor rotamer 21%), 7.30 (d, J=7.4 Hz, NH major rotamer 79%), 6.35 (b, NH major rotamer 84%), 6.13 (b, NH minor rotamer 16%), 4.74 (AB, A Part, J=15.4 Hz, 1H), 4.44 (AB, B part, J=15.4 Hz, 1H), 4.28 (m, 2H), 3.45 (m, 2H), 2.72 (d, J=4.8 Hz, 3H), 2.41 (dA-B, A part, J=7.4 Hz, 14.3 Hz, 1H), 2.31 (dAB, B part, J=5.3 Hz, 14.3 Hz, 1H), 2.23 (m, 1H), 1.98 (m, 2H), 1.88 (m, 1H), 1.43 (s, 9H), 1.25 (d, J=6.8 Hz, 3H).

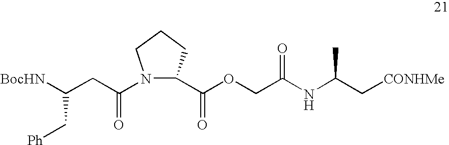

Compound 21. Compound 20 (52.1 mg, 0.140 mmol) was dissolved in 1 mL 4 M HCl/dioxane and the solution was stirred for 1 hour at room temperature. HCl/dioxane was removed in a stream of nitrogen and the residue dried in vacuo. The deprotected depsipeptide and BOC-homophenylalanine (42.5 mg, 0. 152 mmol) were dissolved in 5 mL methylene chloride. DMAP (32.4 mg, 0.265 mmol) was added, followed by EDCI (59.4 mg, 0.310 mmol). After stirring at room temperature for 2 days the solvent was removed in a stream of nitrogen. The residue was dissolved in 0.1 M HCl, and the solution was extracted three times with methylene chloride. The combined organic extracts were dried over MgSO₄ and concentrated to give a colorless glass that was purified by chromatography (silica gel, CHCl₃/MeOH 19:1) to yield 62.8 mg (0.118 mmol, 84%) of 21. ¹H-NMR (300 MHz, CDCl₃) 7.36 (b, NH), 7.31-7.12 (m, 5H), 6.43 (b, NH), 5.22 (b, NH), 4.82 (AB, A part, J=14.9 Hz, 1H), 4.49 (m, 2H), 4.41 (AB, B part, J=15.6 Hz, 1H), 4.21 (m, 1H), 3.52 (m, 1H), 3.32 (m, 1H), 2.89 (m, 1H), 2.78 (m, 1H), 2.71 (d, J=4.8 Hz, 3H), 2.46 (m, 3H), 2.40 (m, 1H), 2.24 (m, 1H), 2.12-1.89 (m, 3H), 1.38 (s, 9H), 1.25 (d, J=6.8 Hz, 3H).

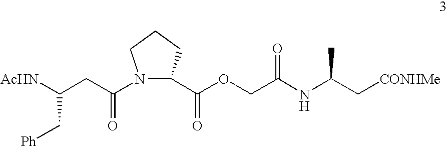

Compound 3. Compound 21 (62.8 mg, 0.118 mmol) was dissolved in 1mL 4 M HCl/dioxane and the solution was stirred for 1 hour at room temperature. HCl/dioxane was removed in a stream of nitrogen and the residue dried in vacuo. The deprotected depsipeptide and triethylamine (90 μL, 0.646 mmol) were dissolved in 1 mL methylene chloride. At 0° C. acetic anhydride (35 μL, 0.371 mmol) was added and the solution was stirred 1 hour at 0° C. and then allowed to warm to room temperature with stirring overnight. The solvent was removed in a stream of nitrogen and the residue dried in vacuo. The residue was purified by chromatography (silica gel, CHCl₃/MeOH 19:1) to yield 52.1 mg (0.110 mmol, 93%) of 3. mp. 128-129° C., ¹H-NMR (300 MHz, CDCl₃) 7.49 (d, J=8.5 Hz, NH), 7.31-7.16 (m, 5H), 6.80 (d, J=8.5 Hz, NH), 6.35 (m, NH), 4.73 (AB, A part, J=15.1 Hz, 1H), 4.49 (m, 1H), 4.45 (AB, B part, J=15.4 Hz, 1H), 4.38 (m, 2H), 3.55 (m, 1H), 3.28 (m, 1H), 2.99 (dAB, A part, J=6.2 Hz, 13.4 Hz, 1H), 2.88 (dAB, B part, J=8.5 Hz, 13.6 Hz, 1H), 2.72 (d, J=4.8 Hz, 3H), 2.55 (dAB, A part, J=5.2 Hz, 15.6 Hz, 1H), 2.43 (dAB, B part, J=5.9 Hz, 15.5 Hz, 1H), 2.45 (d, J=6.3 Hz, 2H), 2.25 (m, 1H), 2.21-1.89 (m, 3H), 1.93 (s, 3H), 1.26 (d, J=6.6 Hz, 3H), ¹³C-NMR (75.5 MHz CDCl₃) 170.90 (C), 170.31 (C), 169.75 (C), 128.93 (CH), 128.32 (CH), 126.40 (CH), 62.60 (CH₂), 58.89 (CH), 47.31 (CH₂), 42.44 (CH, CH₂), 39.57 (CH₂), 36.22 (CH₂),28.92 (CH₂), 25.96 (CH₃), 24.94 (CH₂), 23.02 (CH₃), 20.21 (CH₃), IR (1 mM in CH₂Cl₂) 3452, 3433, 3346 cm^{b-1}, El MS m/e 474.2474 calc. for C₂₄H₃₄N₄O₆ 474.2478.

Construction of polypeptides using any type of beta-amino acid can be accomplished using conventional and widely recognized solid-phase or solution-phase synthesis. Very briefly, in solid-phase synthesis, the desired C-terminal amino acid residue is linked to a polystyrene support as a benzyl ester. The amino group of each subsequent amino acid to be added to the N-terminus of the growing peptide chain is protected with Boc, Fmoc, or another suitable protecting group. Likewise, the carboxylic acid group of each subsequent amino acid to be added to the chain is activated with DCC and reacted so that the N-terminus of the growing chain always bears a removable protecting group. The process is repeated (with much rinsing of the beads between each step) until the desired polypeptide is completed. In the classic route, the N-terminus of the growing chain is protected with a Boc group, which is removed using trifluoracetic acid, leaving behind a protonated amino group. Triethylamine is used to remove the proton from the N-terminus of the chain, leaving a the free amino group, which is then reacted with the activated carboxylic acid group from a new protected amino acid. When the desired chain length is reached, a strong acid, such as hydrogen bromide in trifluoracetic acid, is used to both cleave the C-terminus from the polystyrene support and to remove the N-terminus protecting group.

The preferred solid-phase synthesis used herein is shown in Reaction 7: 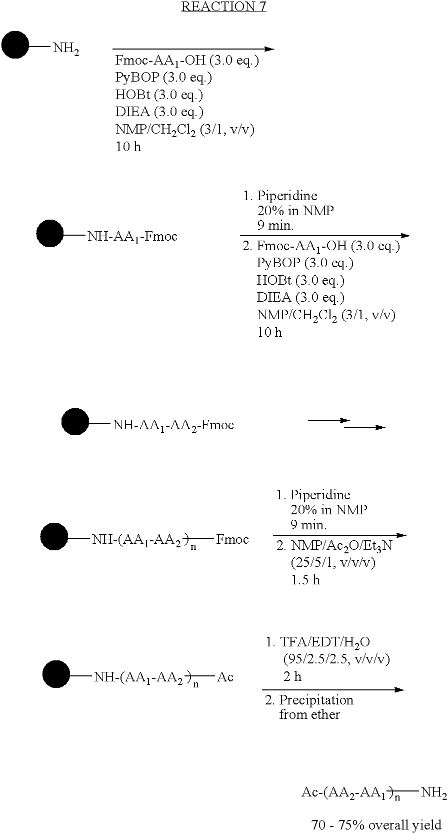
AAₙ=incoming amino acid to be added to chain
Fmoc=the protecting group 9-fluorenylmethyloxycarbonyl
NMP=N-methyl pyrrolidone
EDT=Ethanedithiol
PyBOP=benzotriazol-1 -yloxytripyrrolidinophosphonium hexafluorophosphate
HOBt=N-hydroxy-benzotriazole
DIEA=diisopropylethyl amine
AAₙ=incoming amino acid to be added to chain
Fmoc=the protecting group 9-fluorenylmethyloxycarbonyl
NMP=N-methyl pyrrolidone
EDT=Ethanedithiol
PyBOP=benzotriazol-1 -yloxytripyrrolidinophosphonium hexafluorophosphate
HOBt=N-hydroxy-benzotriazole
DIEA=diisopropylethyl amine

Solid-phase peptide synthesis is widely employed and well know. Consequently, it will not be described in any further detail here. For a contemporary treatment of the Fmoc-based polypeptide synthesis, see W. C. Chan and Peter D. White, “Fmoc Solid Phase Peptide Synthesis, A Practical Approach” copyright 2001, Oxford University Press. For a contemporary and exhaustive treatment of Polypeptide synthesis covering both solid-phase and solution-phase synthesis, see N. L. Benoiton, “Chemistry of Peptide Synthesis,” copyright 2006, CRC Press.

Solution phase synthesis, noted above, can also be used with equal success. For example, solution-phase synthesis of a beta-peptide chain containing alternating residues of unsubstituted cyclohexane rings and amino-substituted cyclohexane rings proceeds in conventional fashion as outlined in Reaction 8:

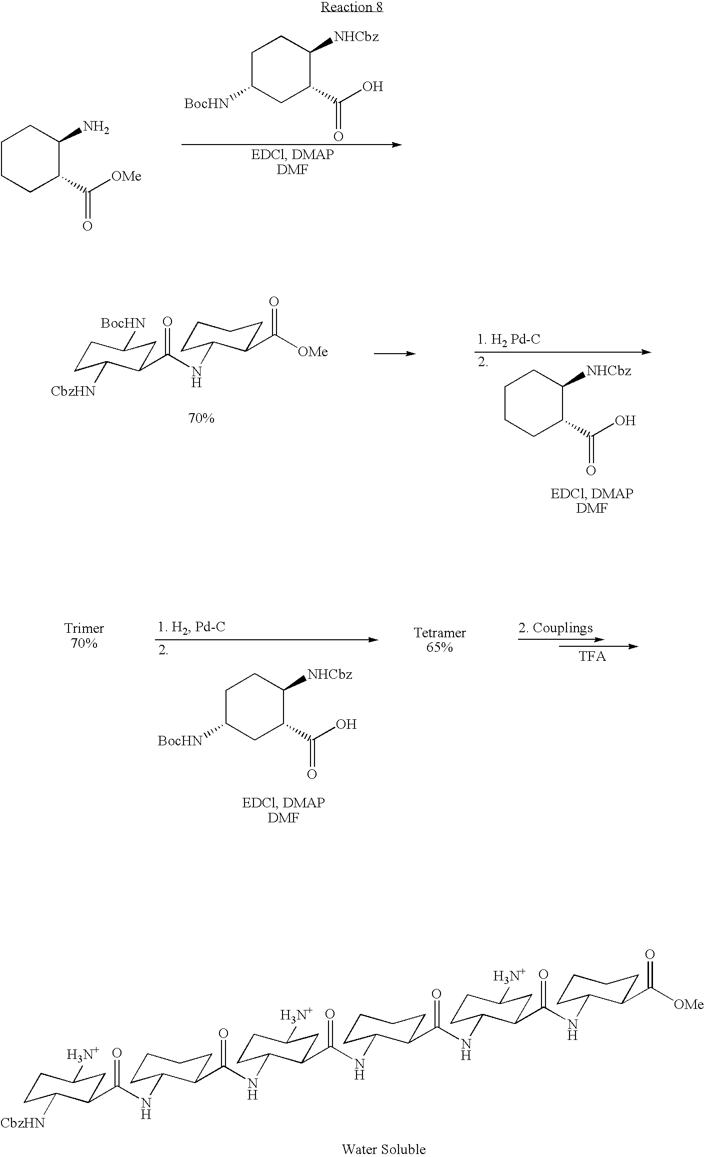

Reaction 8 works with equal success to build peptides wherein the residues are the same or different.

Reaction 9 is an illustration of a homologation reaction combined with conventional solution-phase peptide synthesis which yields a beta-peptide having acyclic-substituted residues alternating with ring-constrained residues:

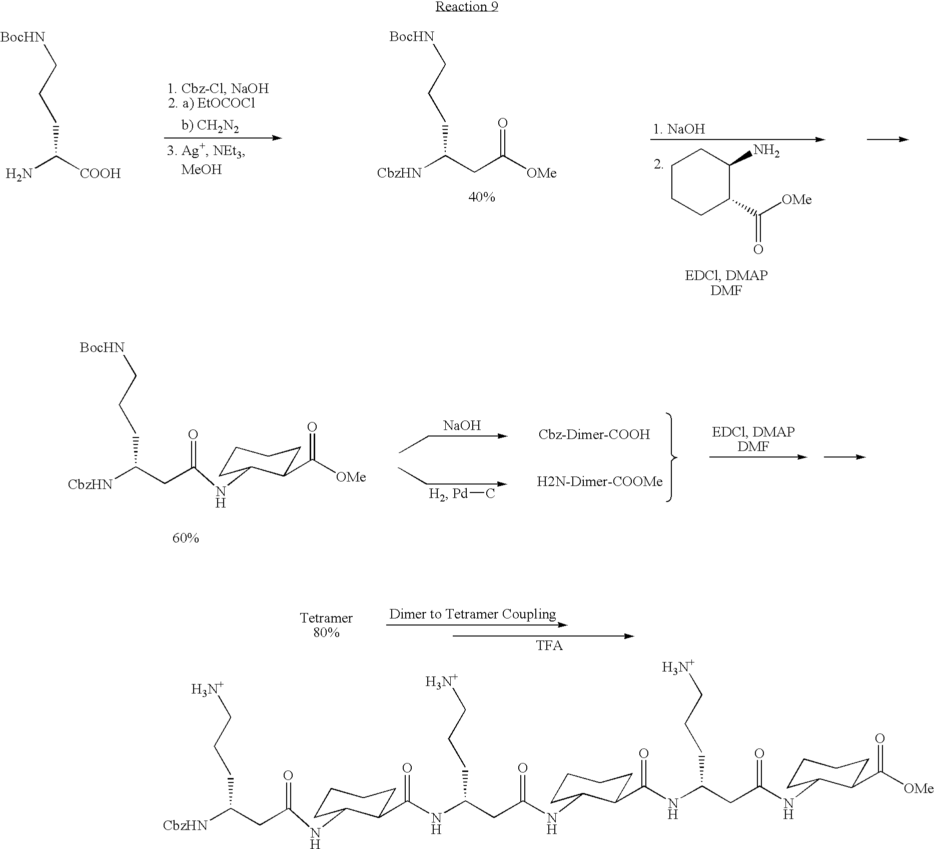

As noted above, the beta-peptides of the present invention can be substituted with any number of substituents, including hydroxy, linear or branched C₁-C₆-alkyl, alkenyl, alkynyl; hydroxy-C₁-C₆-alkyl, amino-C₁-C₆-alkyl, C₁-C₆-alkyloxy, C₁-C₆-alkyloxy-C₁-C₆-alkyl, amino, mono- or di-C₁-C₆-alkylamino, carboxamido, carboxamido-C₁-C₆-alkyl, sulfonamido, sulfonamido-C₁-C₆-alkyl, urea, cyano, fluoro, thio, C₁-C₆-alkylthio, mono- or bicyclic aryl, mono- or bicyclic heteraryl having up to 5 heteroatoms selected from N, O, and S; mono- or bicyclic aryl-C₁-C₆-alkyl, heteroaryl-C₁-C₆-alkyl, and combinations thereof. Effecting such substitutions is well within the set of skills possessed by a synthetic peptide chemist.

For example, appending a sulfonamido moiety to the cylic backbone substituent can be accomplished in conventional fashion using Reaction 10.

Compound 63: Compound 61 (90 mg) was dissolved in 4 N HCl in dioxane (2.0 ml). The reaction mixture was stirred for 1.5 hours. The dioxane was then removed in vacuo. The residue was dissolved in pyridine (2.0 ml), then cooled to 0° C. in an ice-bath.

Methanesulfonylchloride (71 μL) was added dropwise. After the addition, the reaction mixture was stirred at room temperature for 12 hours. The pyridine was then removed in vacuo. The residue was taken up in ethyl acetate (50 ml). The mixture was washed with dilute brine (2×10 ml), dried over MgSO₄, and concentrated to give the clean product as a colorless oil (70 mg) in 82% yield.

Compound 64: Compound 62 (30 mg) was dissolved in 4 N HCl in dioxane (2.0 ml). The reaction mixture was stirred for 1.5 hours. The dioxane was then removed in vacuo. The residue was dissolved in pyridine (1.0 ml), then cooled to 0° C. in an ice-bath. Toluenesulfonylchloride (63 mg) was added in portions. After the addition, the reaction mixture was stirred at room temperature for 12 tours. The pyridine was then removed in vacuo. The residue was taken up in methylene chloride/dithyl ether (1/1, v/v, 100 ml). The mixture was washed with dilute brine (3×20 ml), dried over MgSO₄, and concentrated to give a liquid residue. The crude product was purified by column chromatography with ethyl acetate/hexane (4/6, v/v) as eluent to give the clean product as a colorless oil (25 g) in 74% yield.

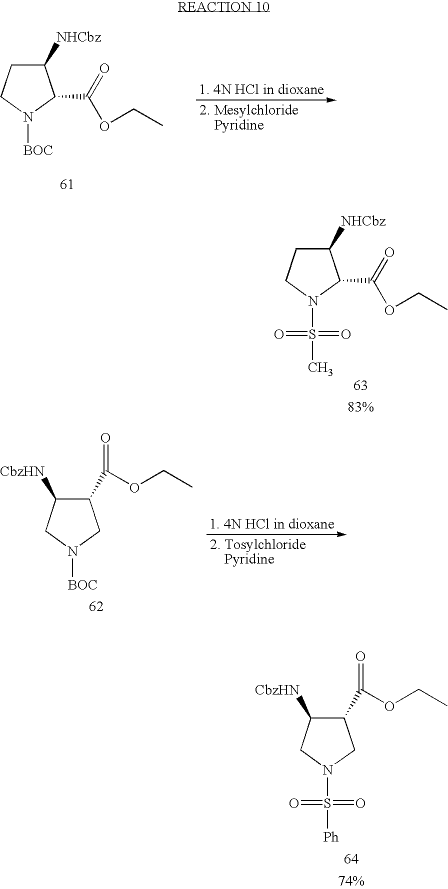

Analogous reactions will append a carboxyamido group.

Using the above-described techniques, as well as convention solid-phase and solution-phase peptide synthesis, a host of first, second, third, and subsequent generations of compounds according to the present invention were fabricated, as detailed below (the left-hand is the compound no, the right-hand column designates whether the compound mimics gB or gH, if known). Several of these compounds were tested for their ability to inhibit HCMV entry into cells, as described in the Examples.

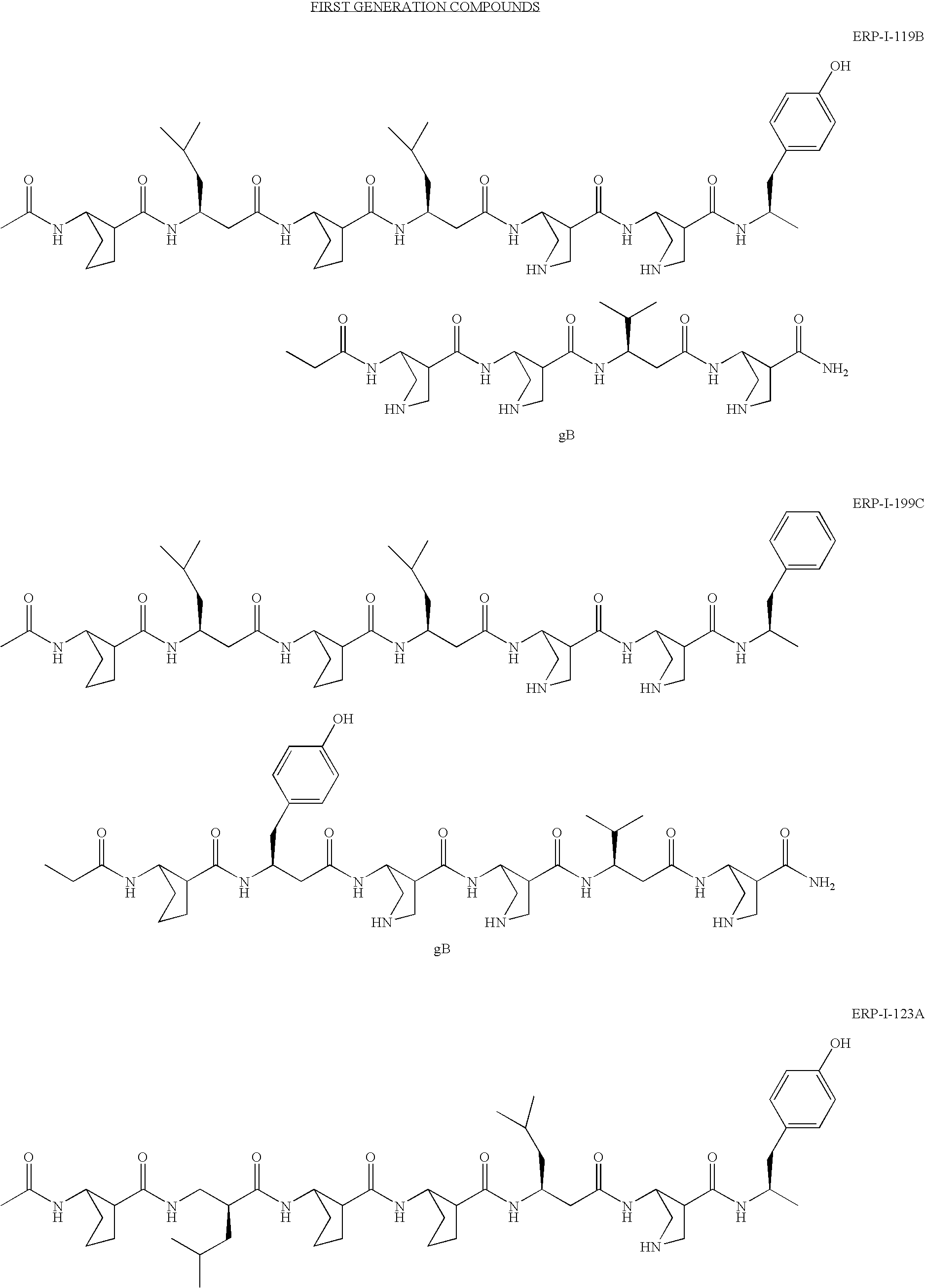

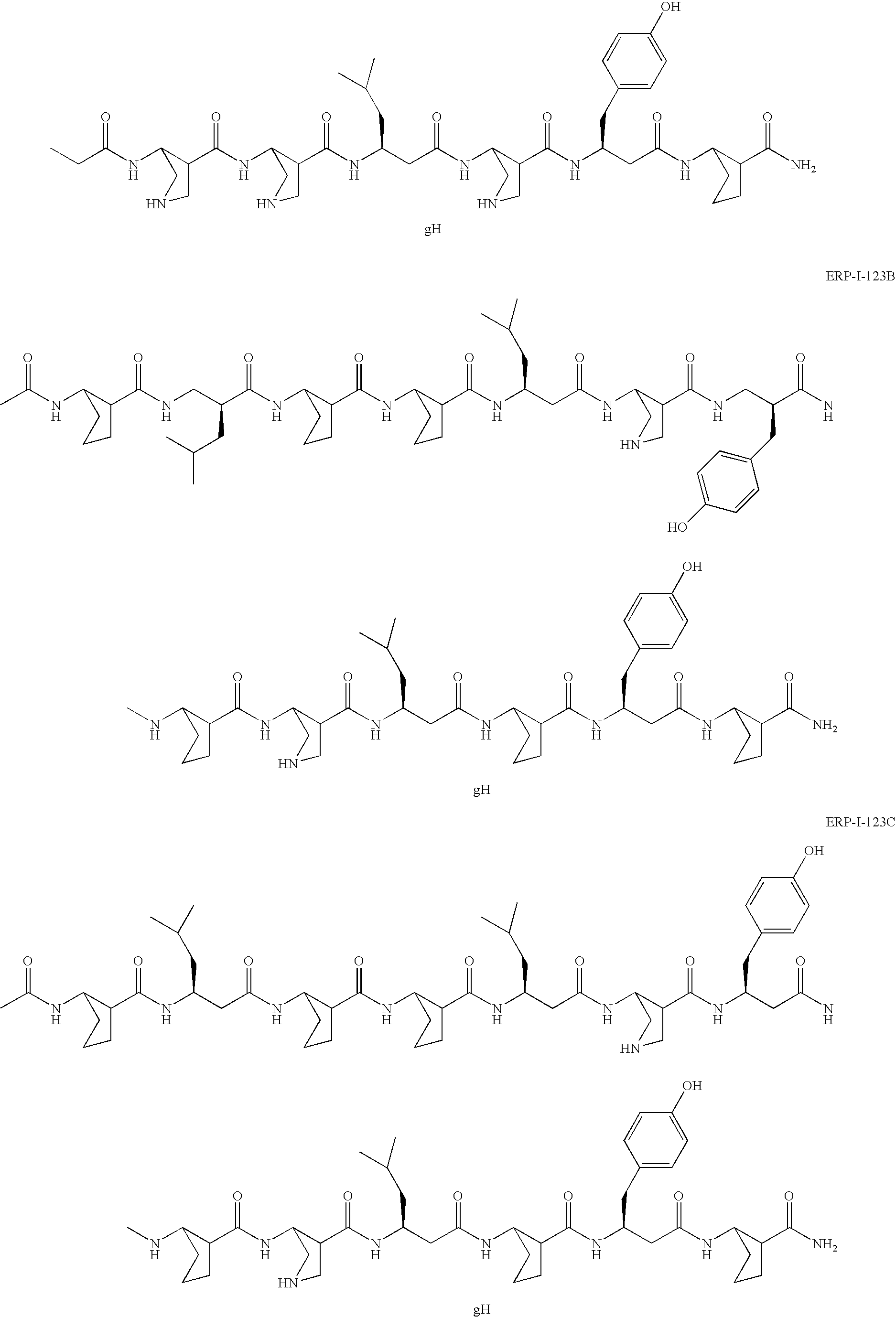

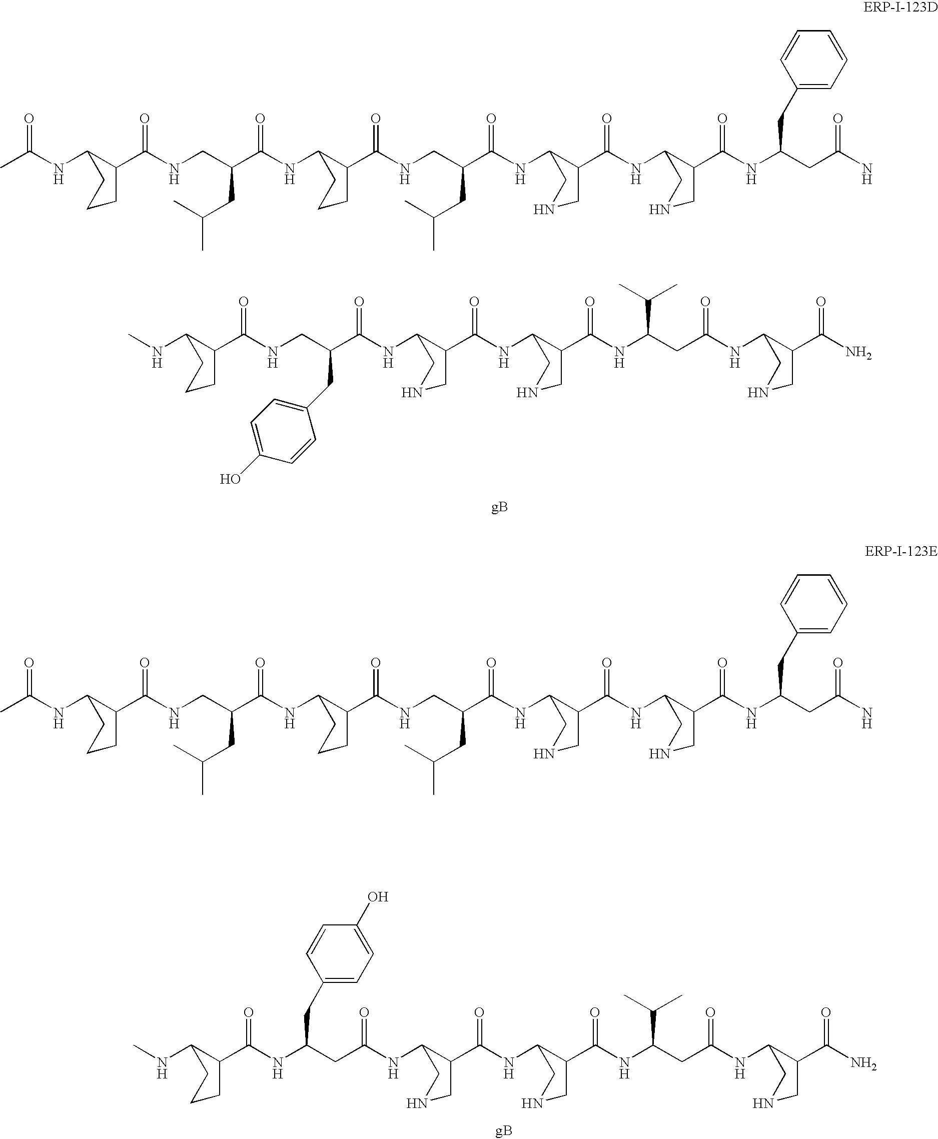

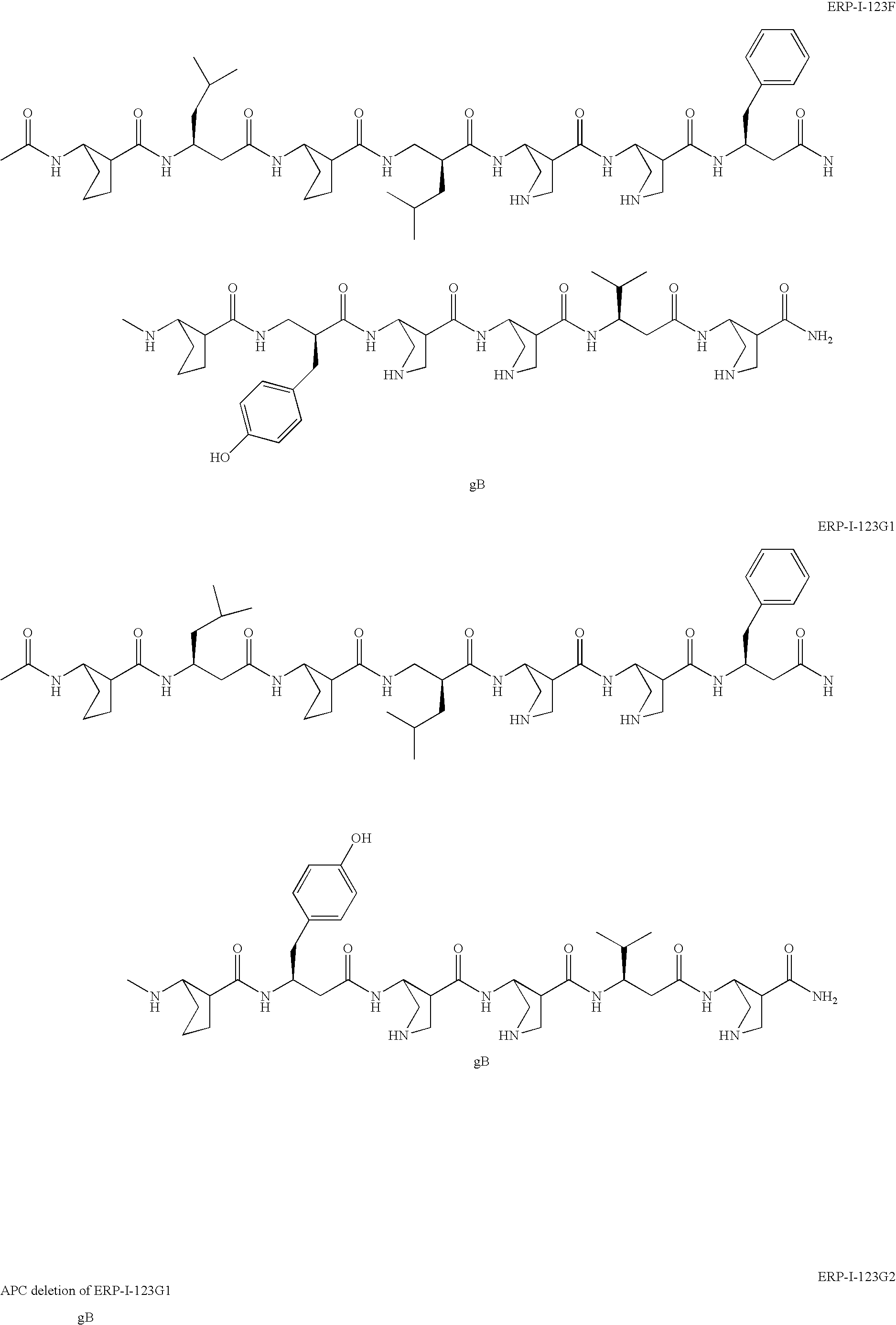

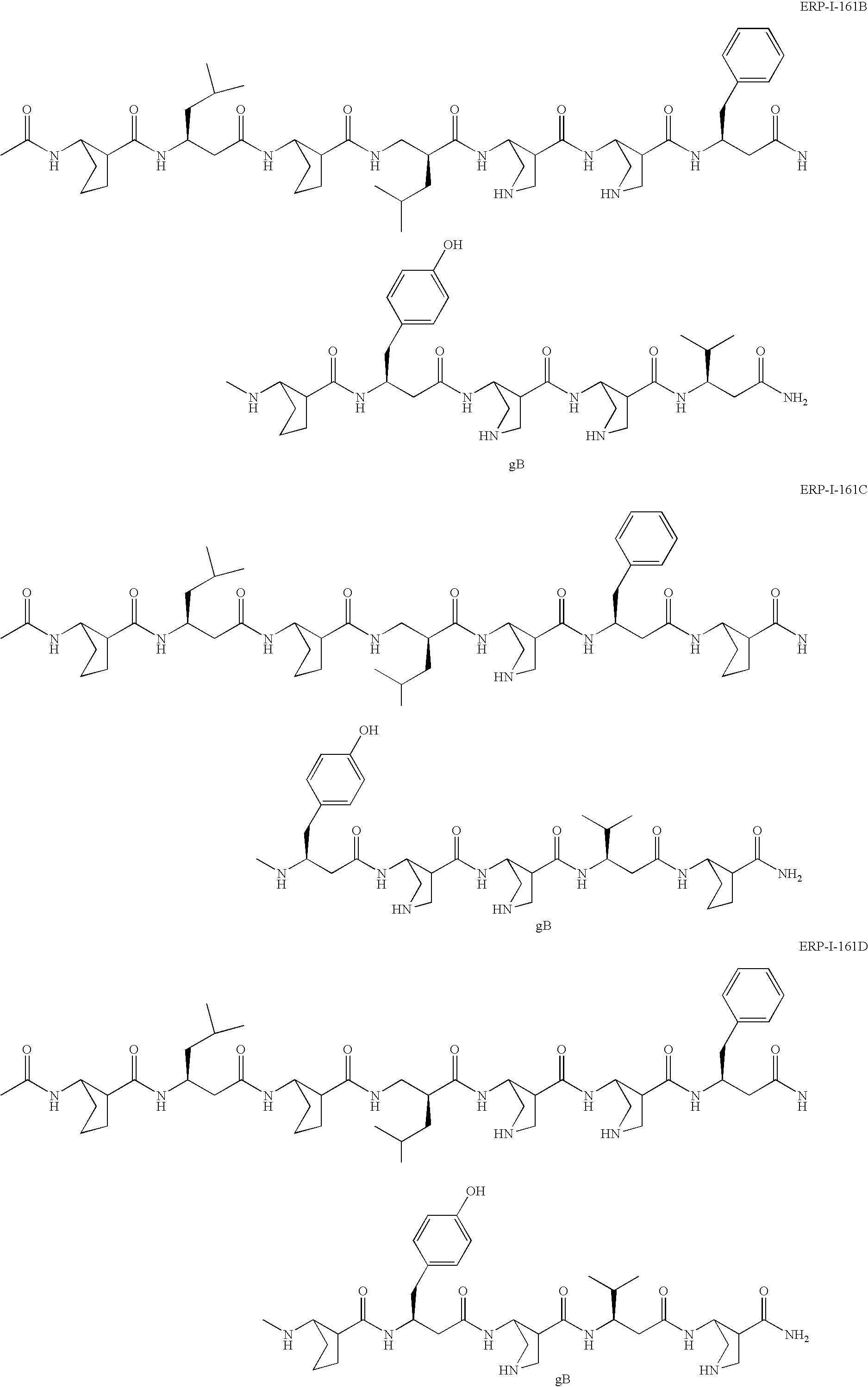

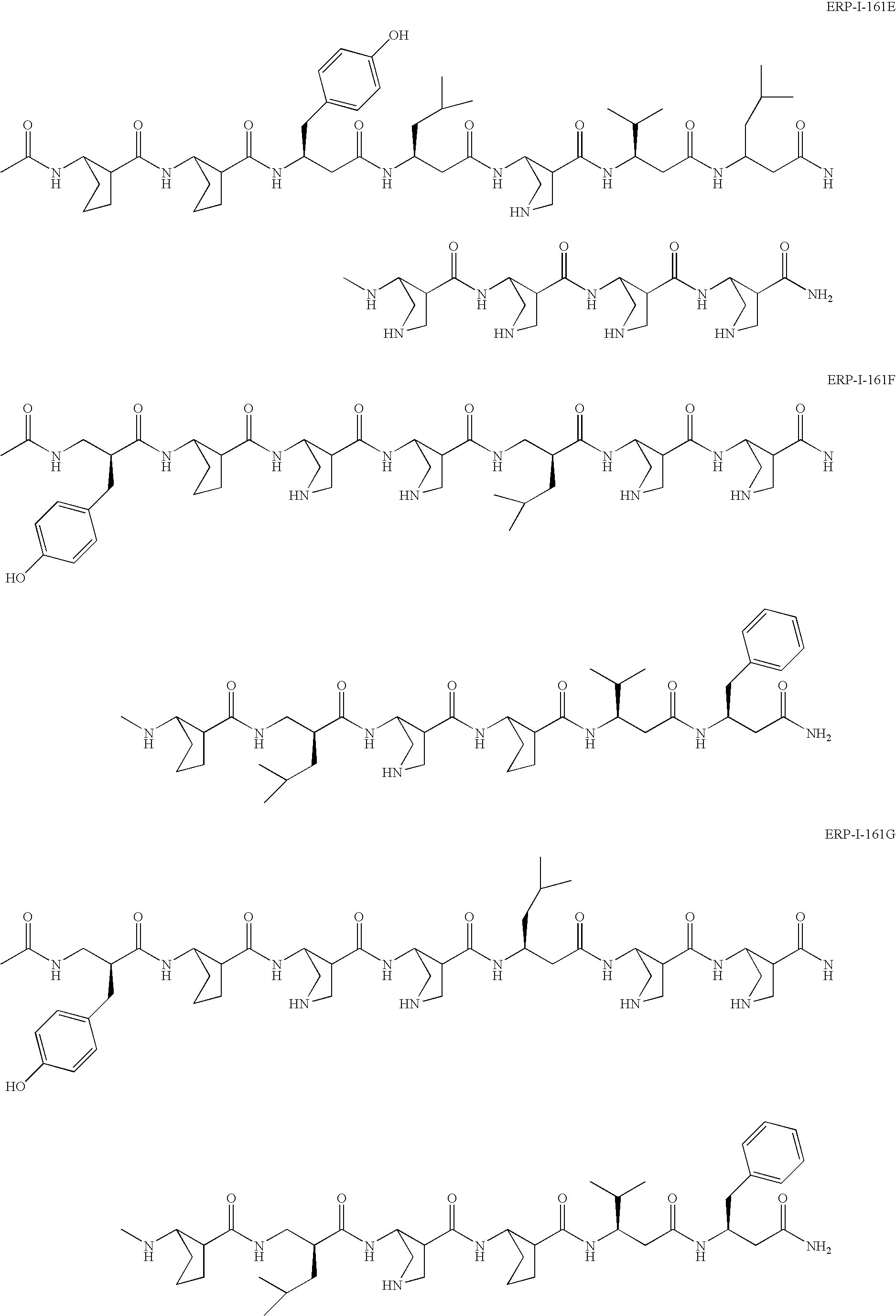

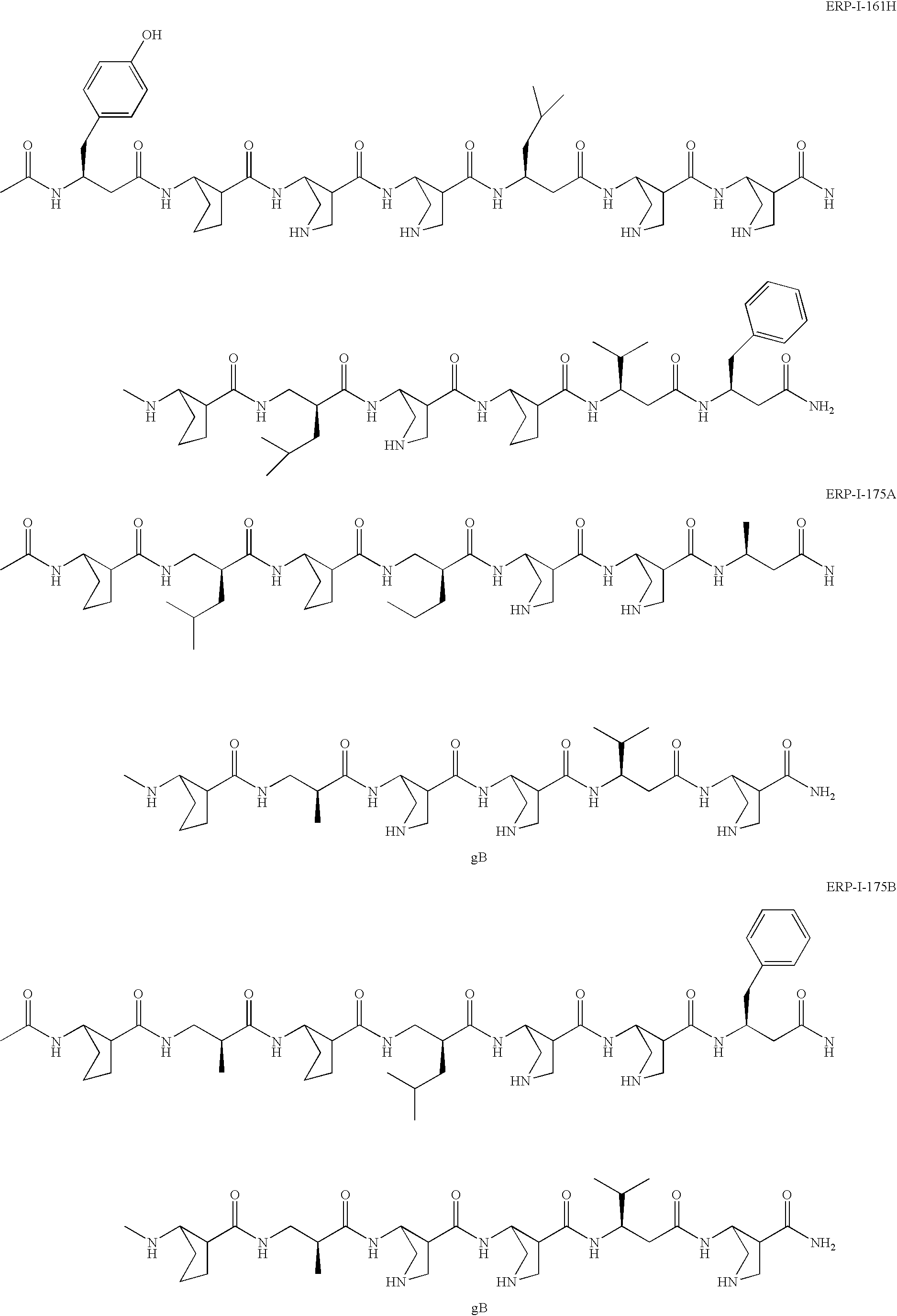

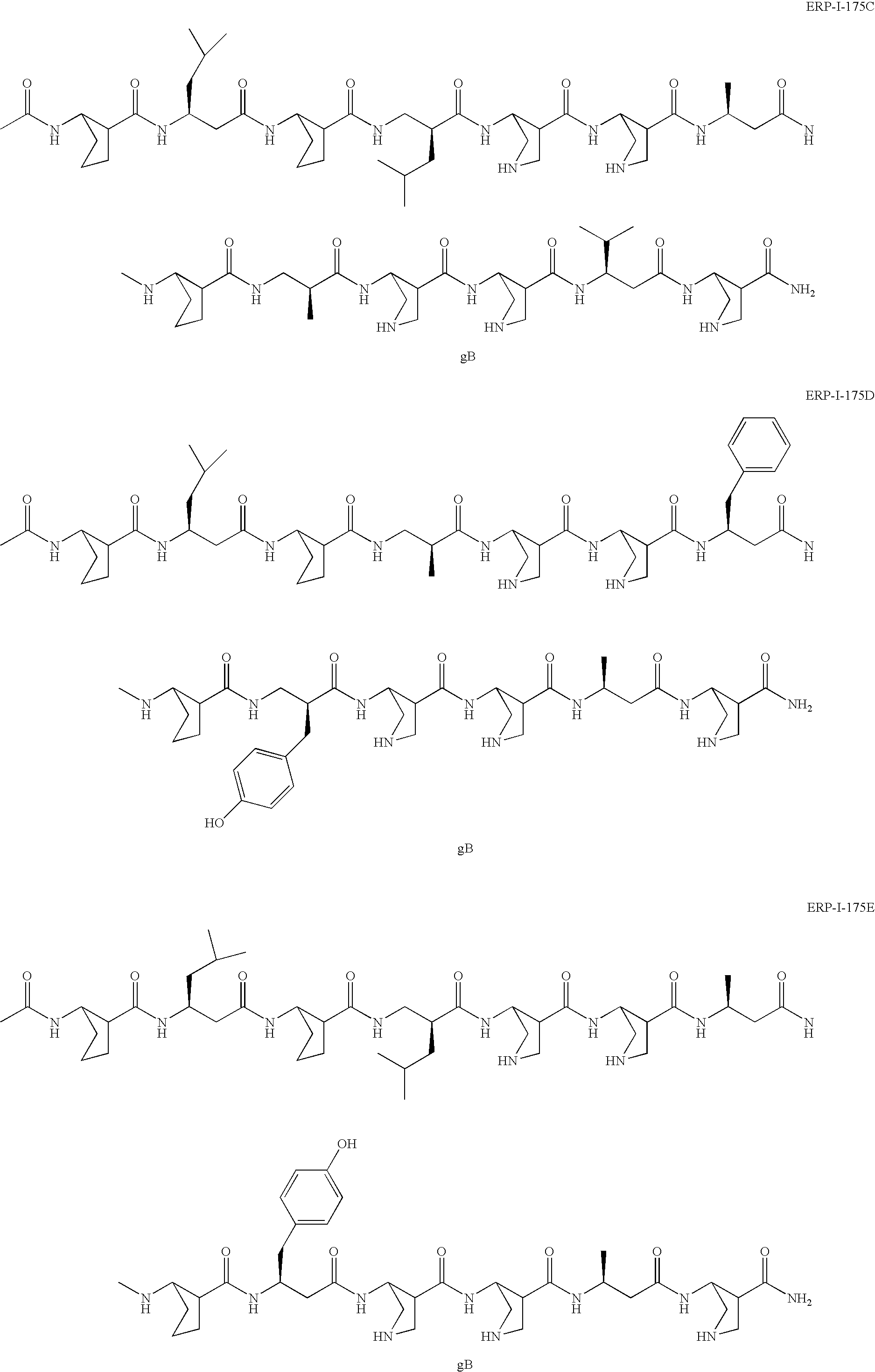

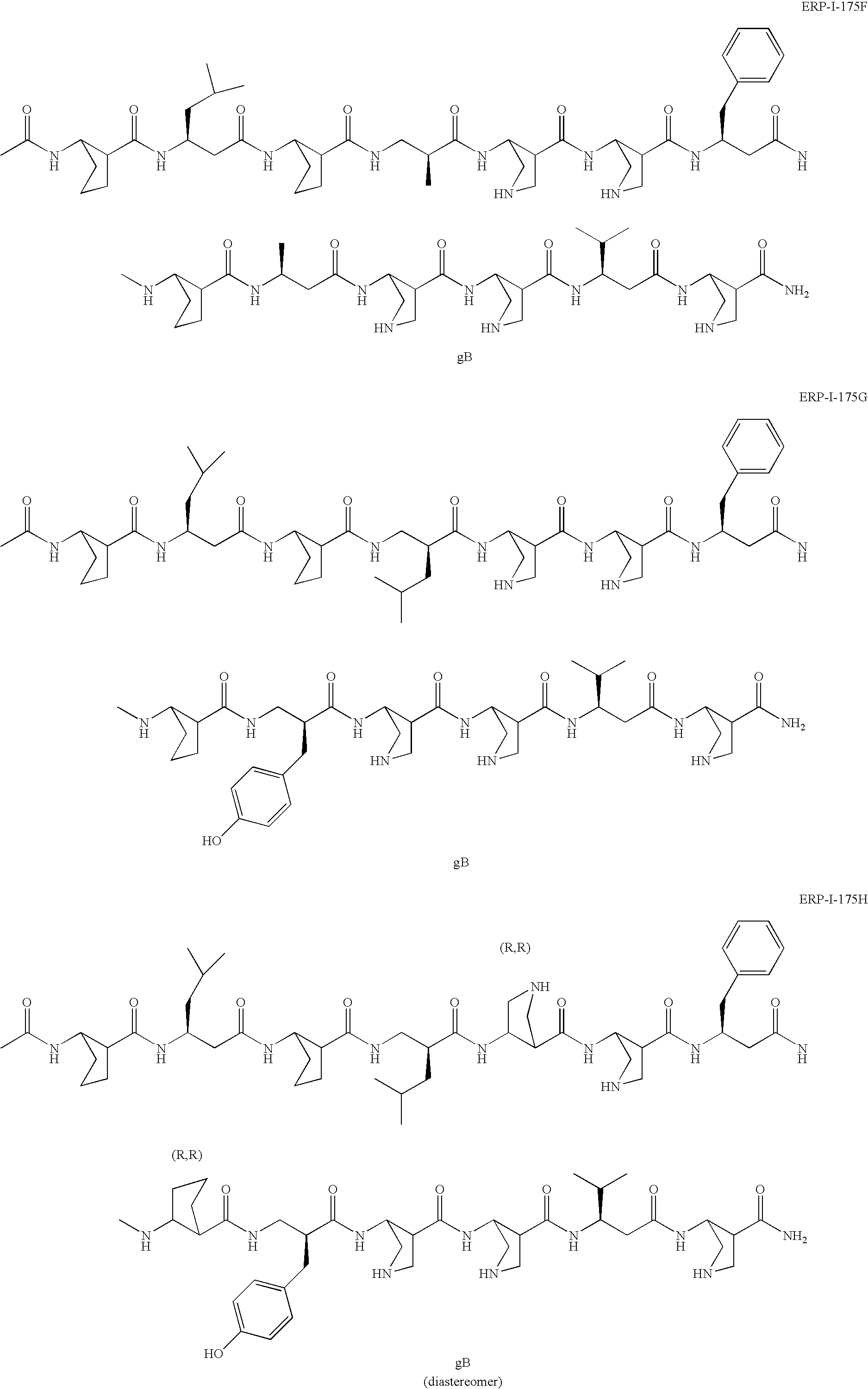

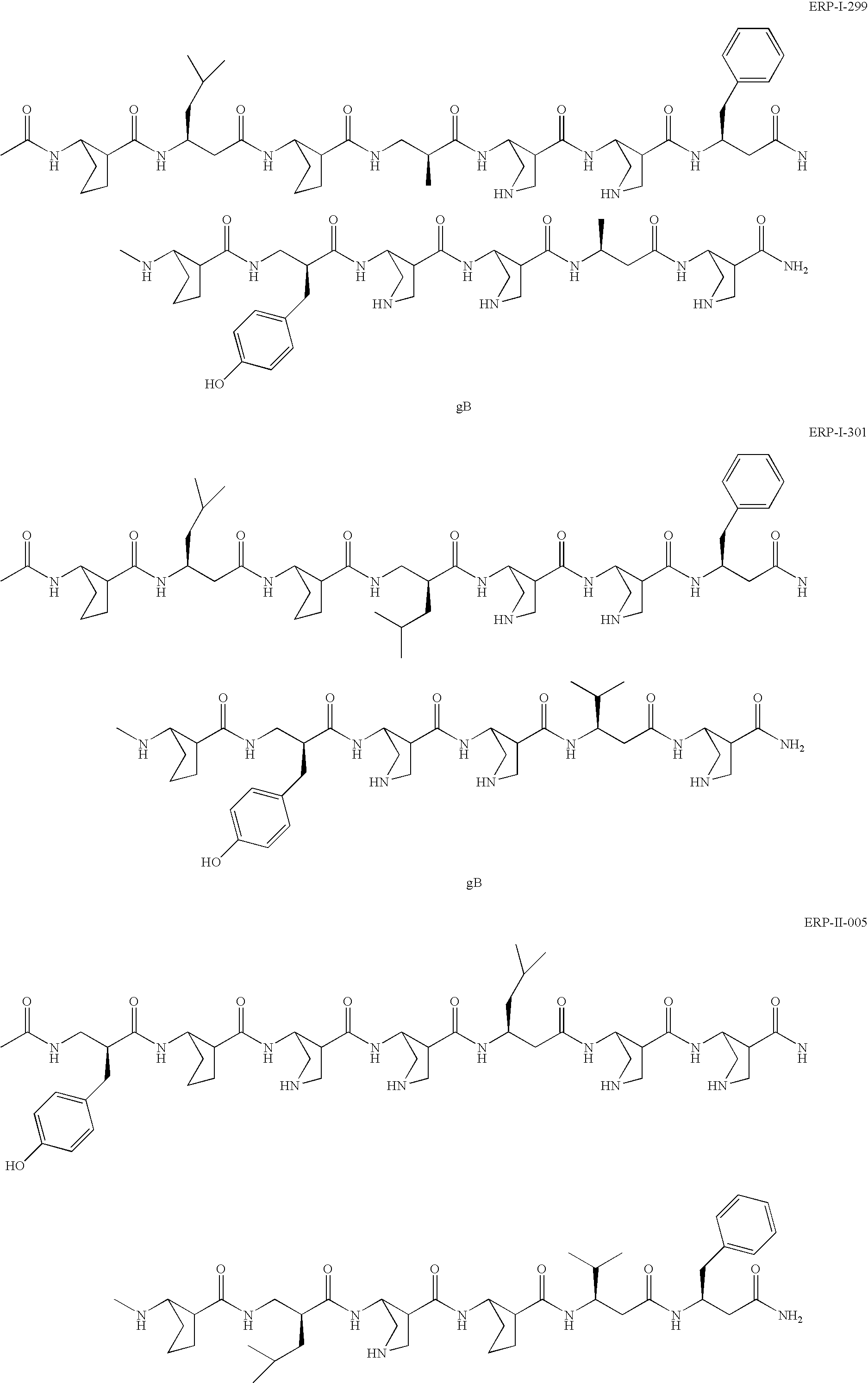

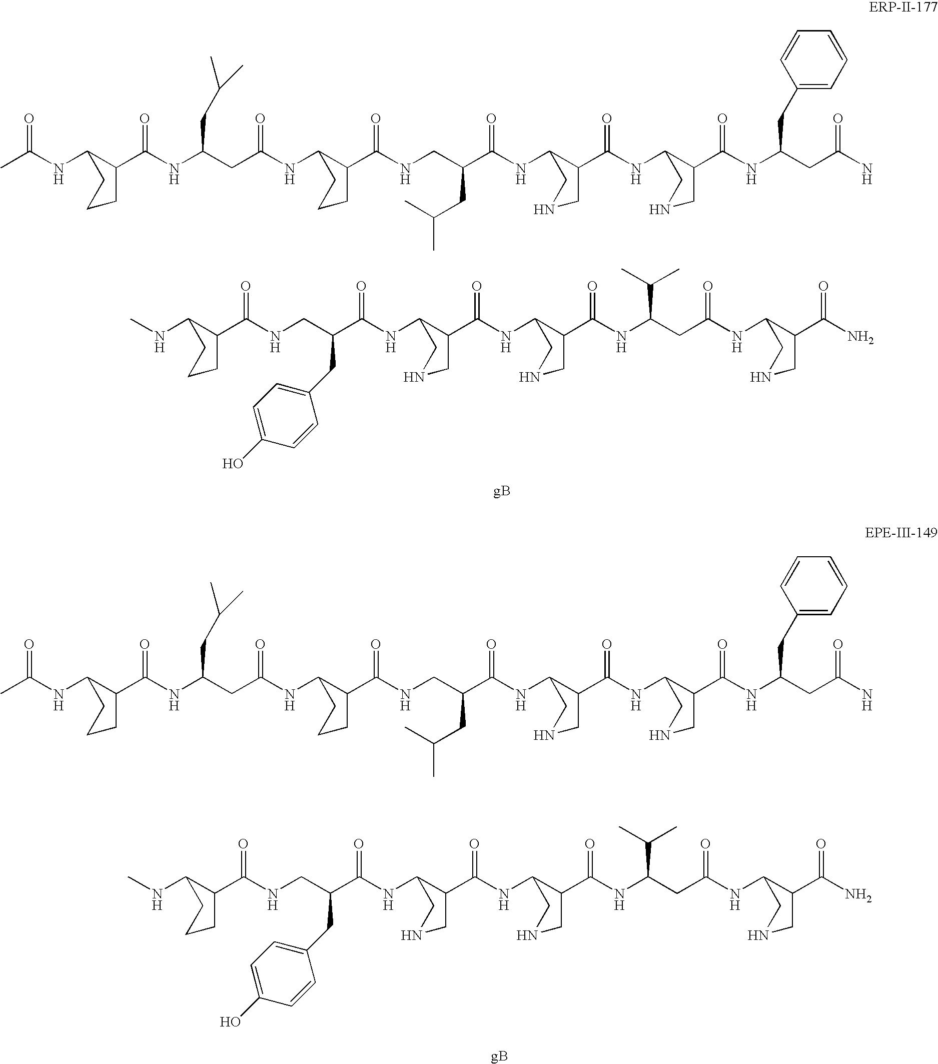

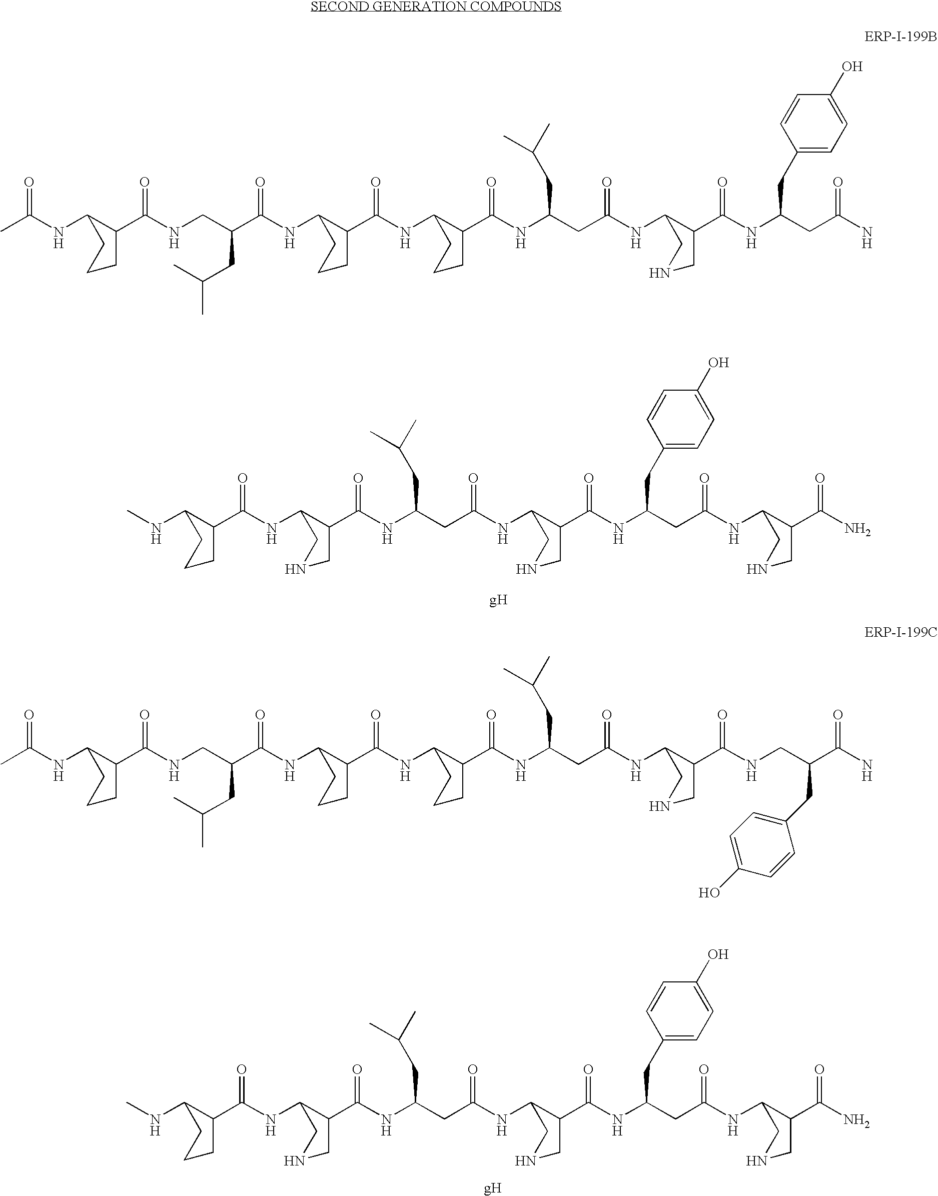

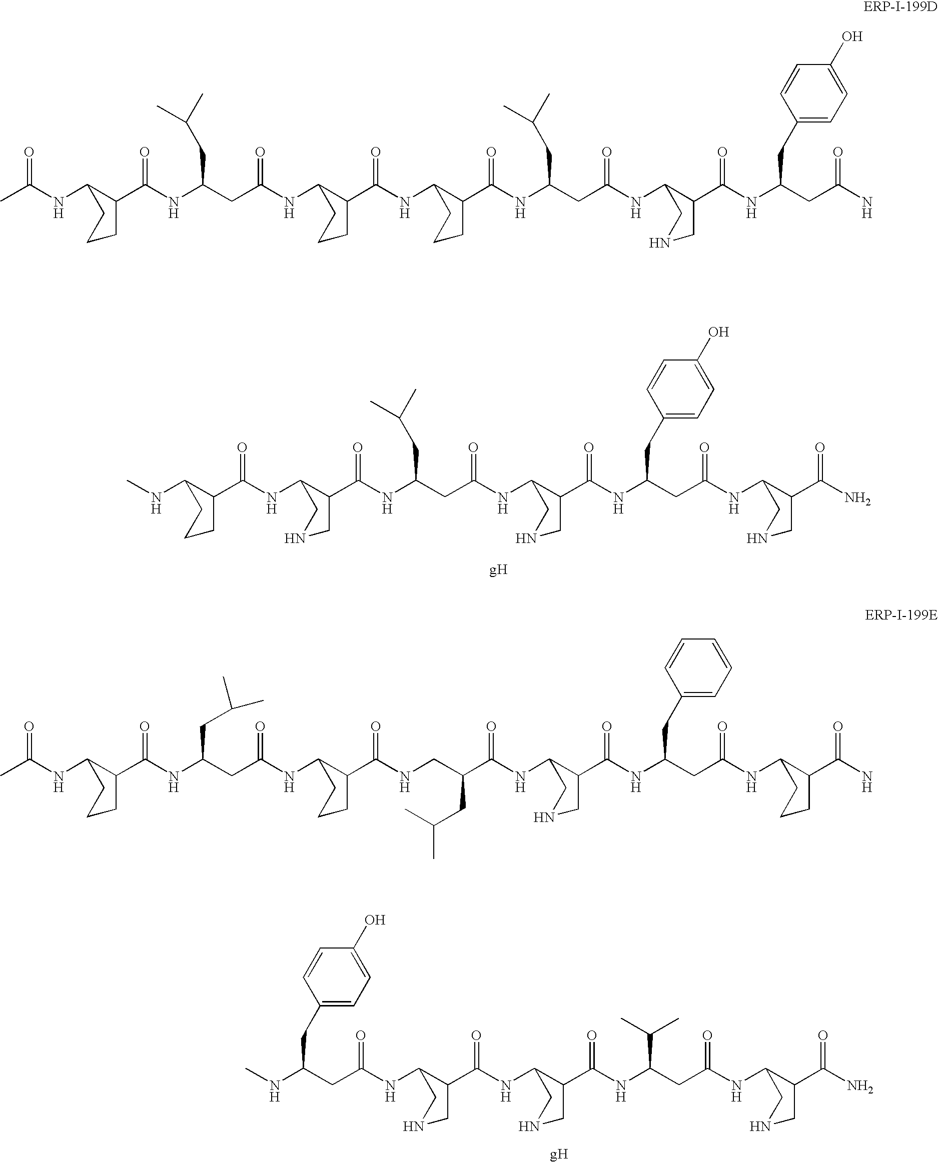

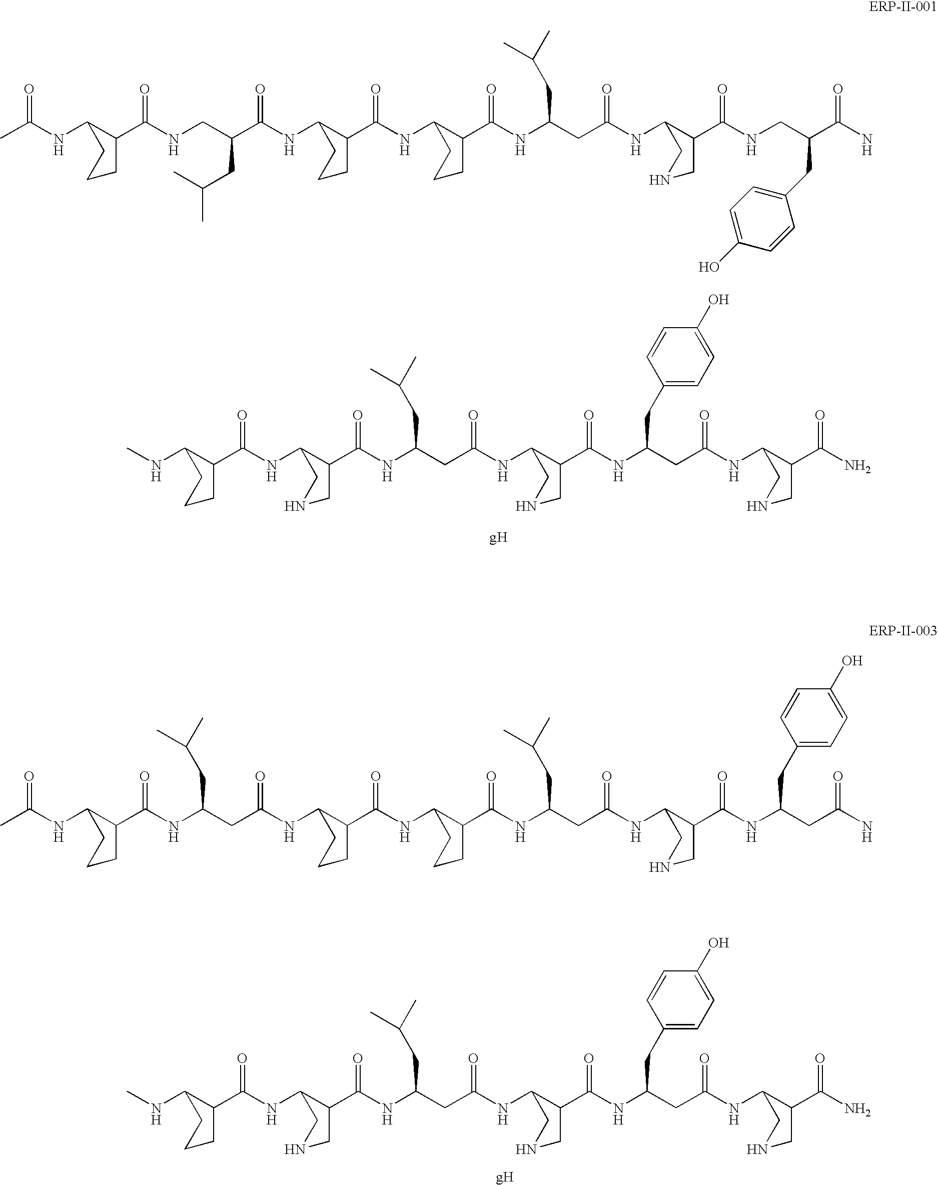

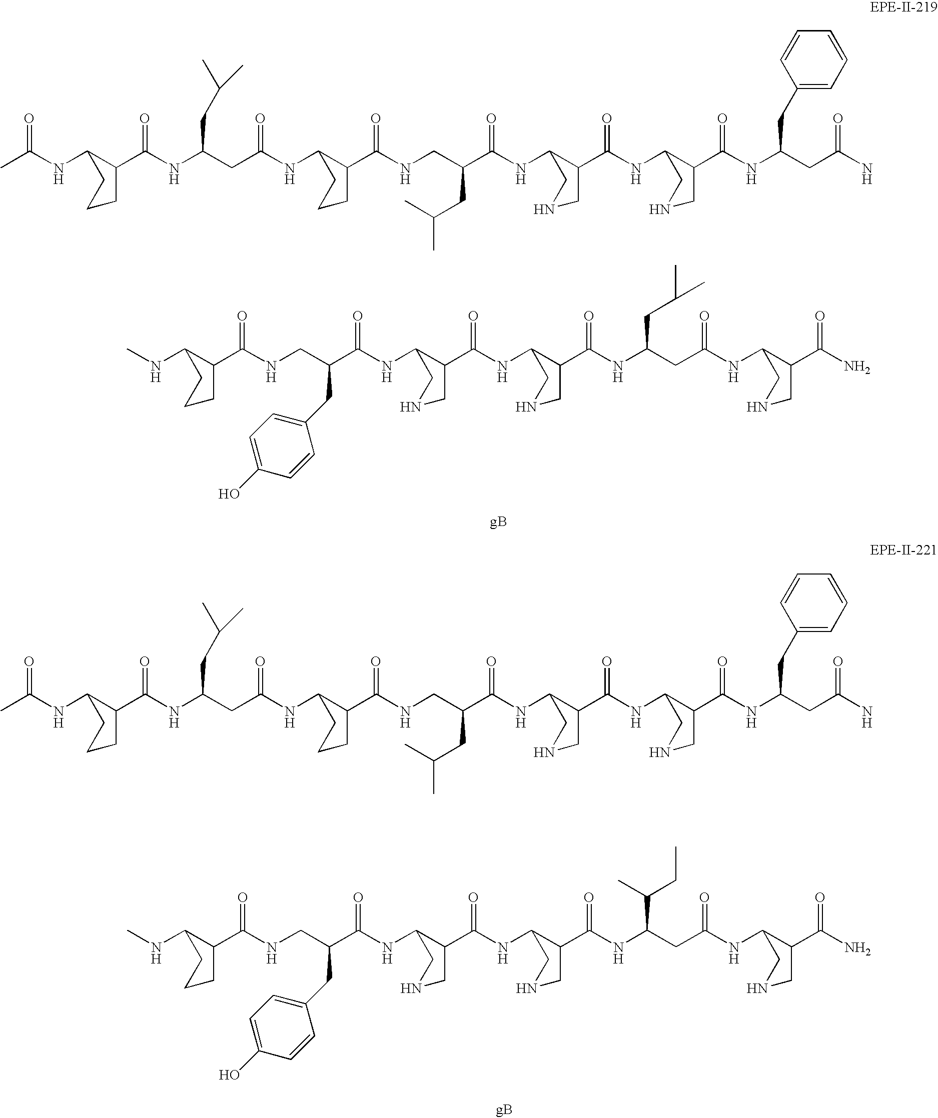

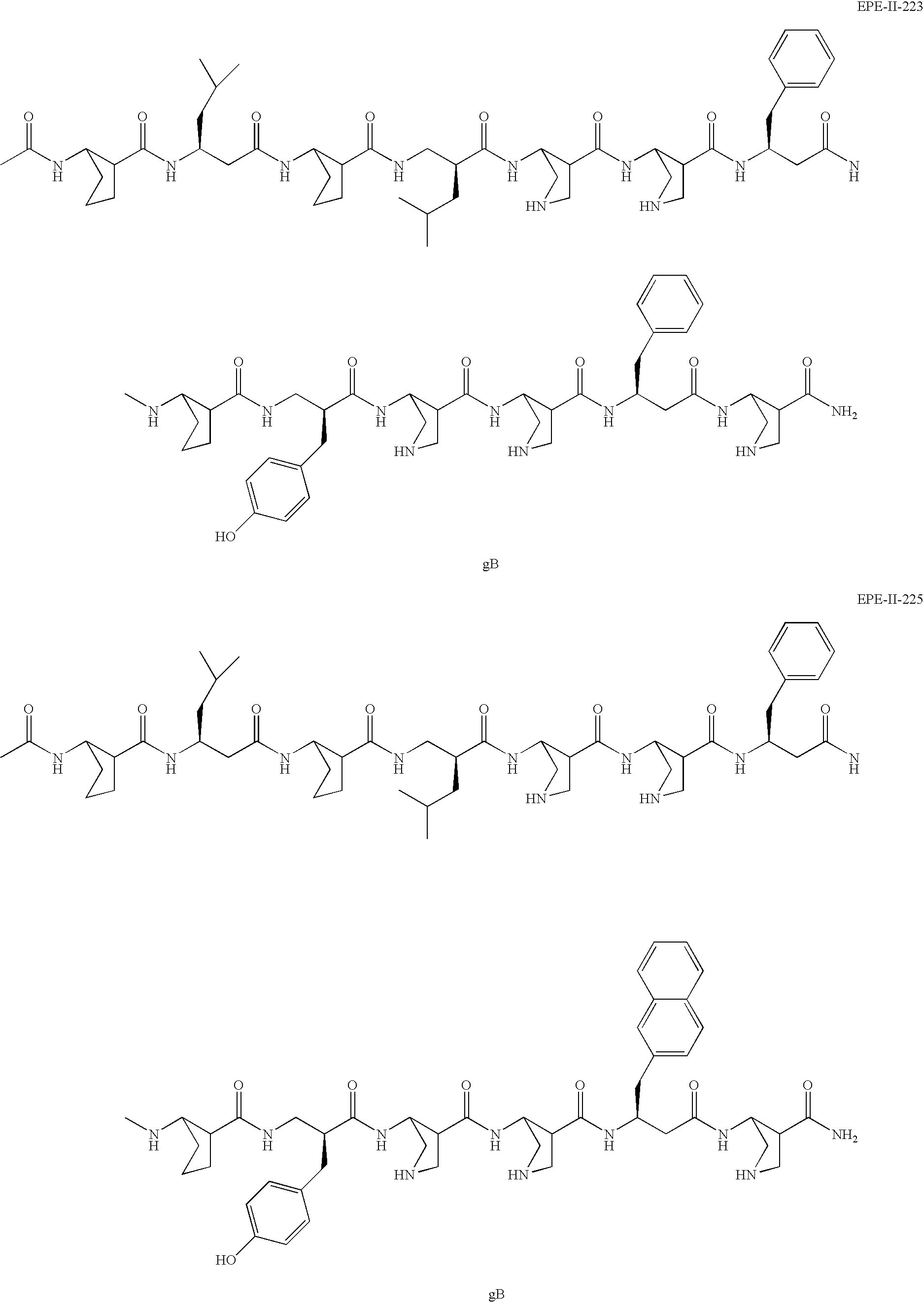

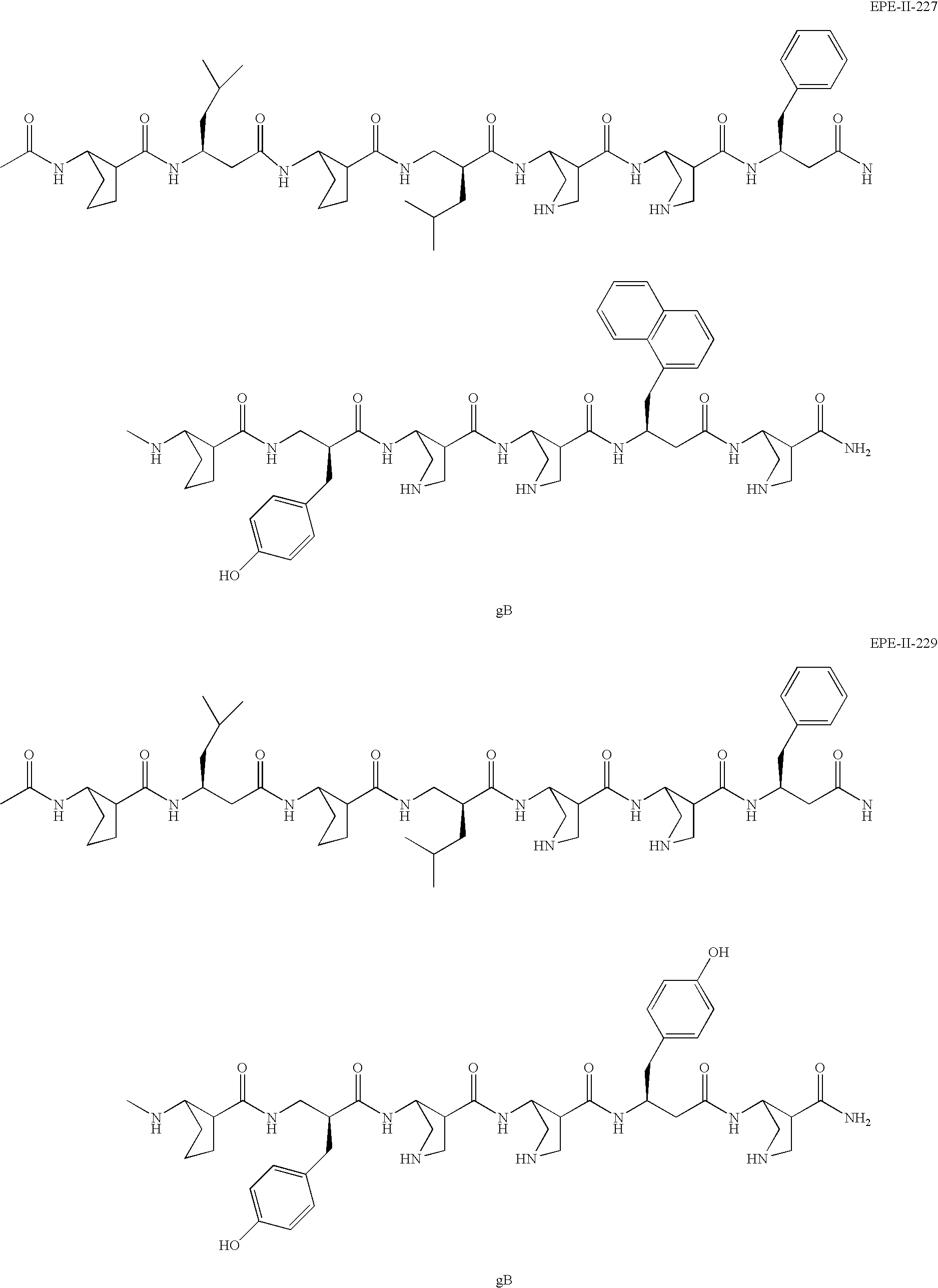

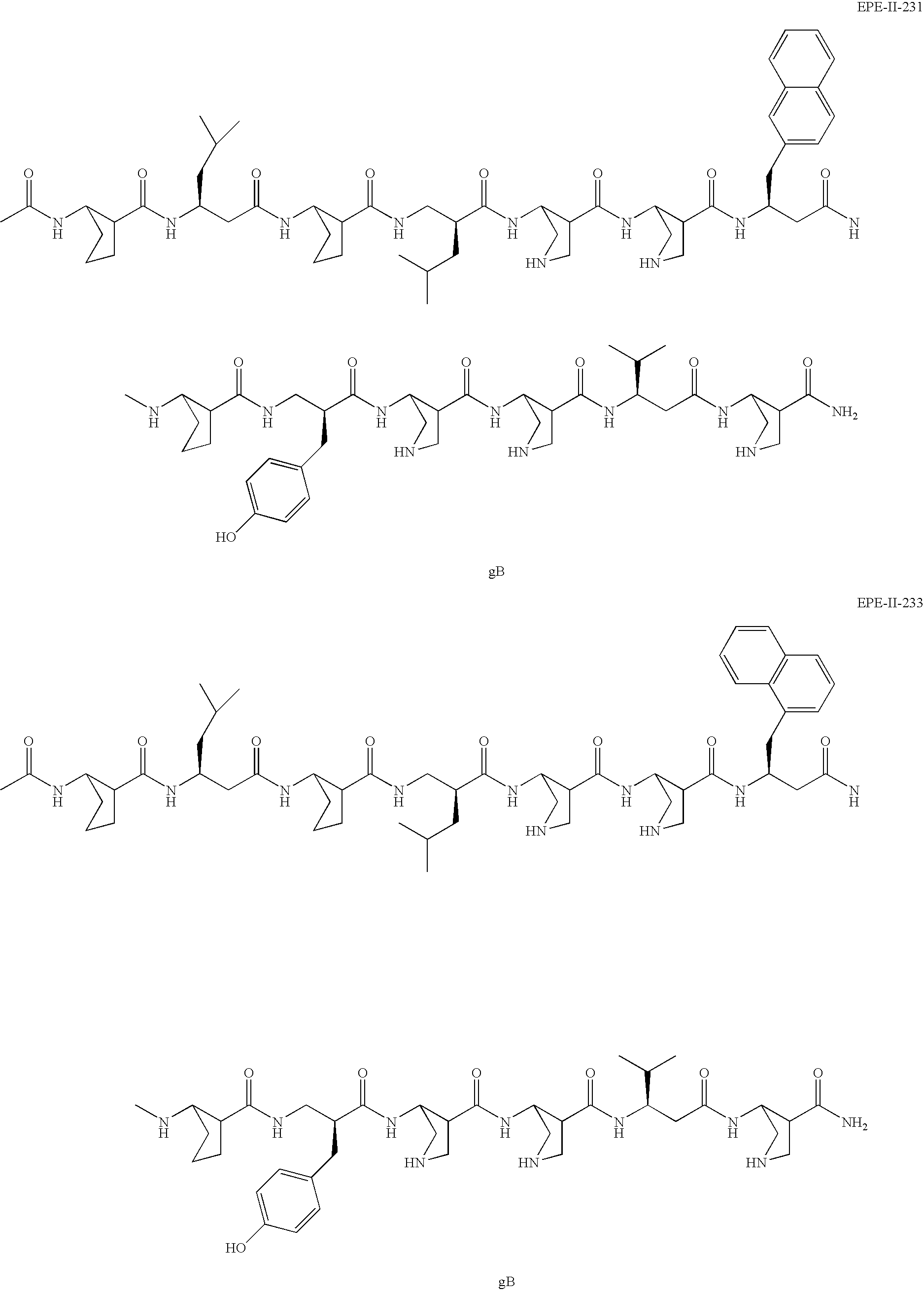

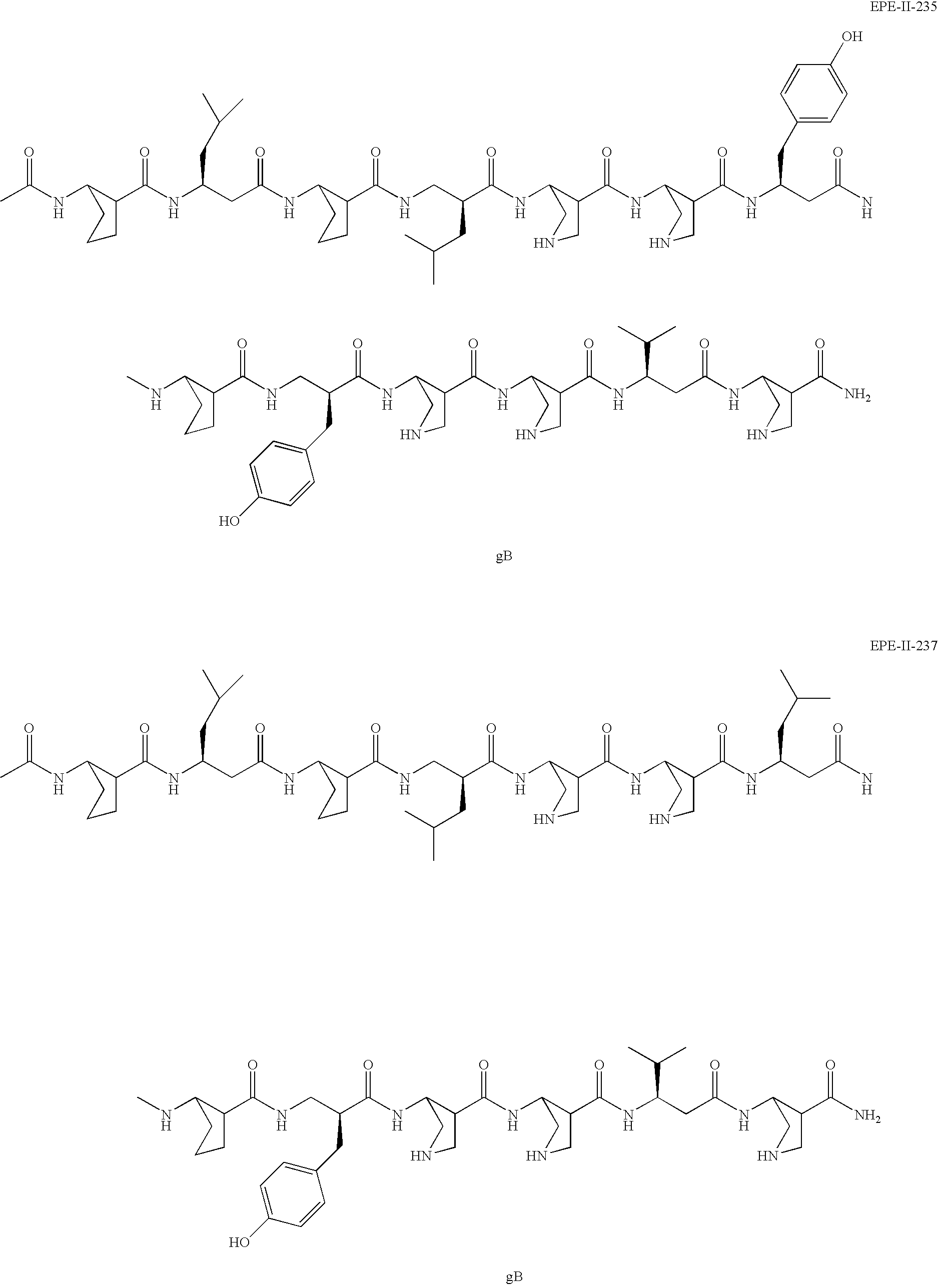

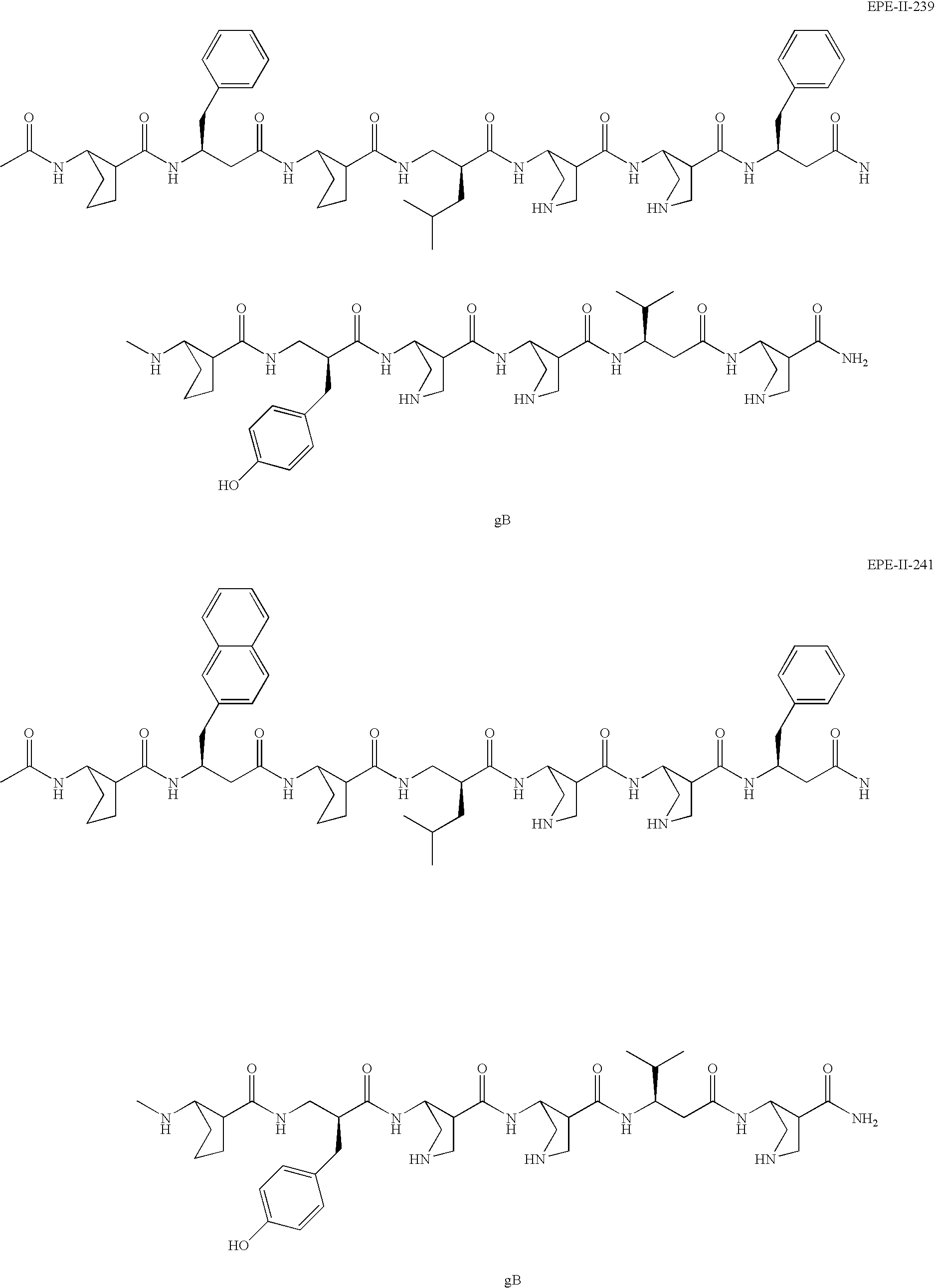

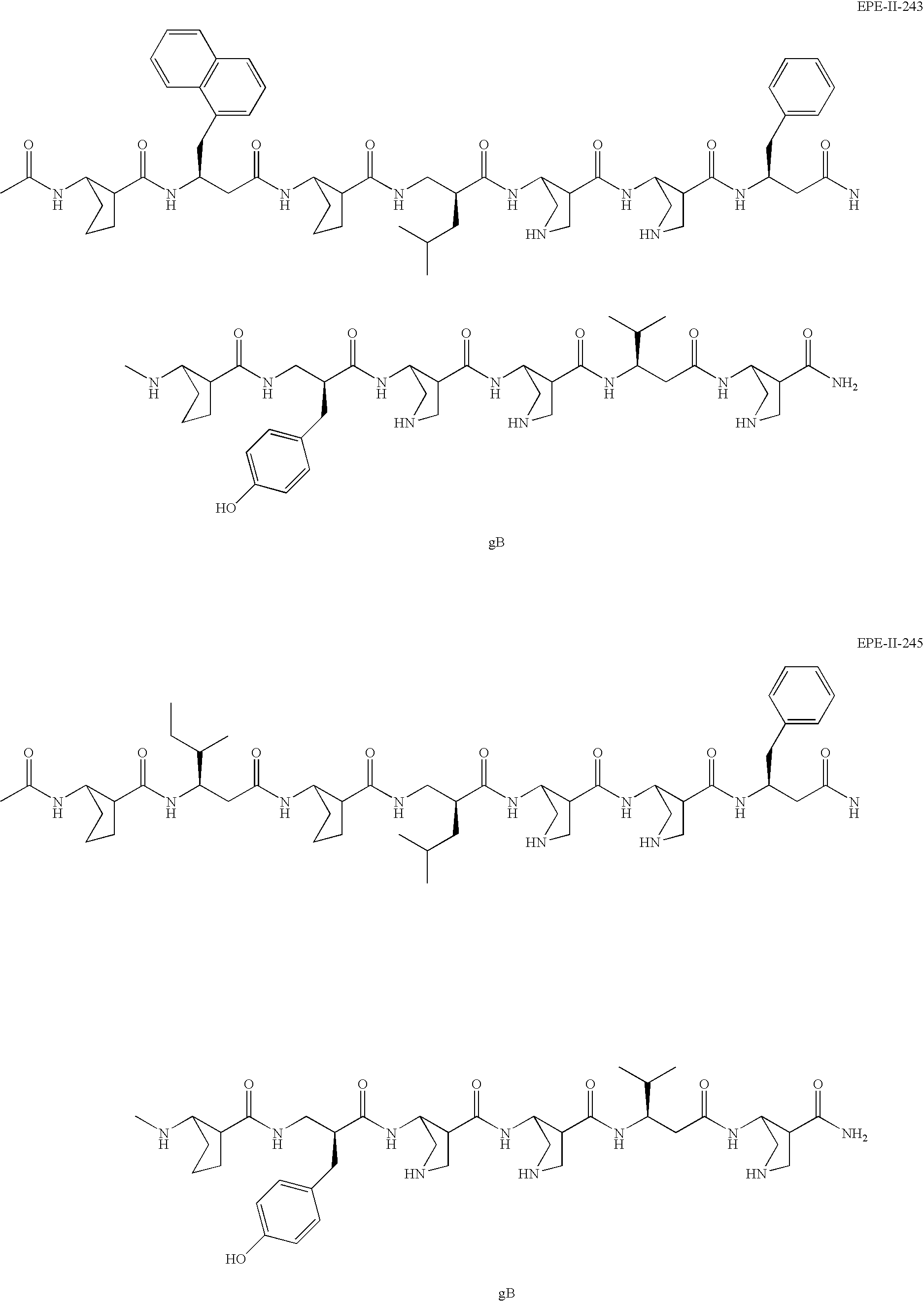

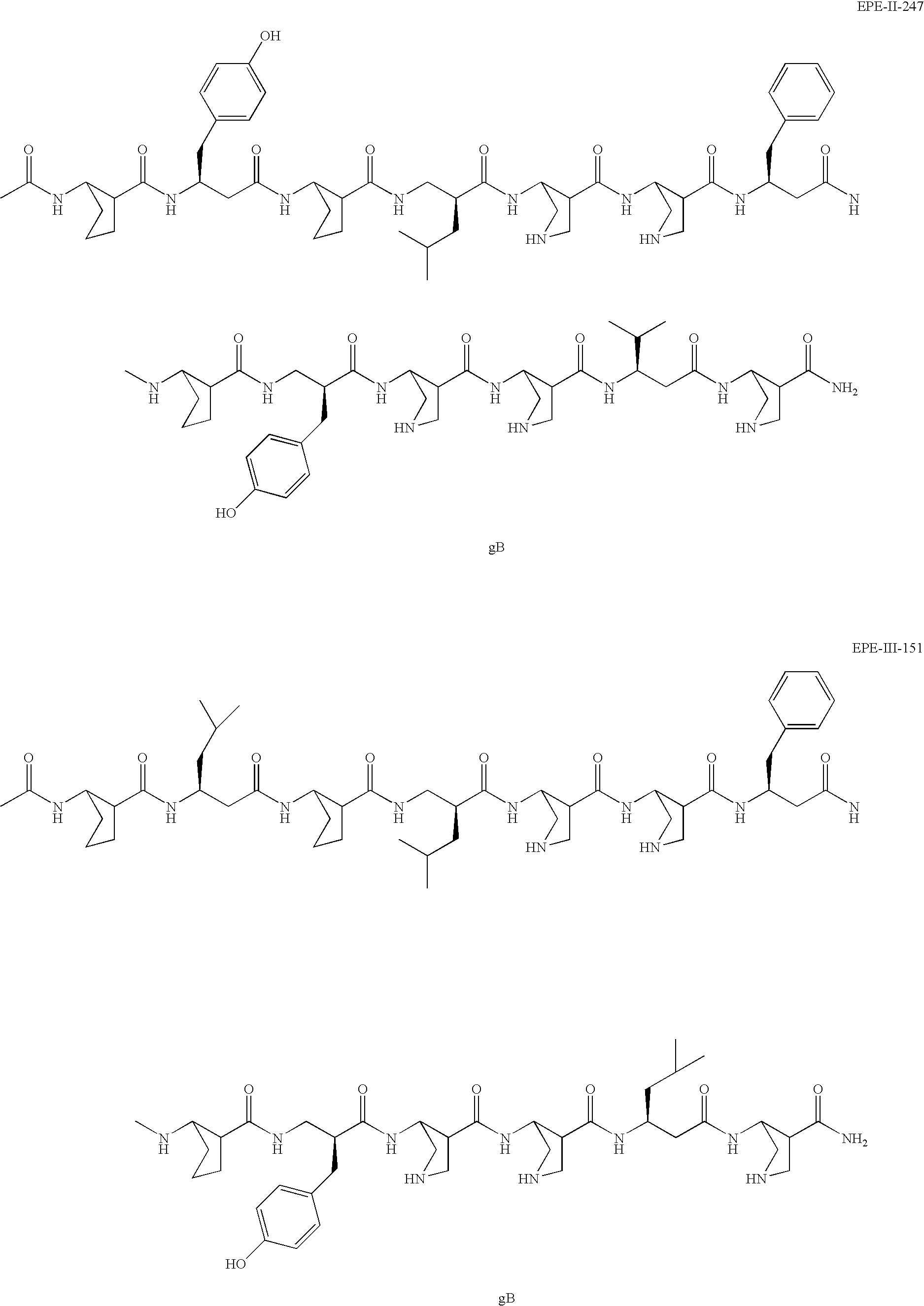

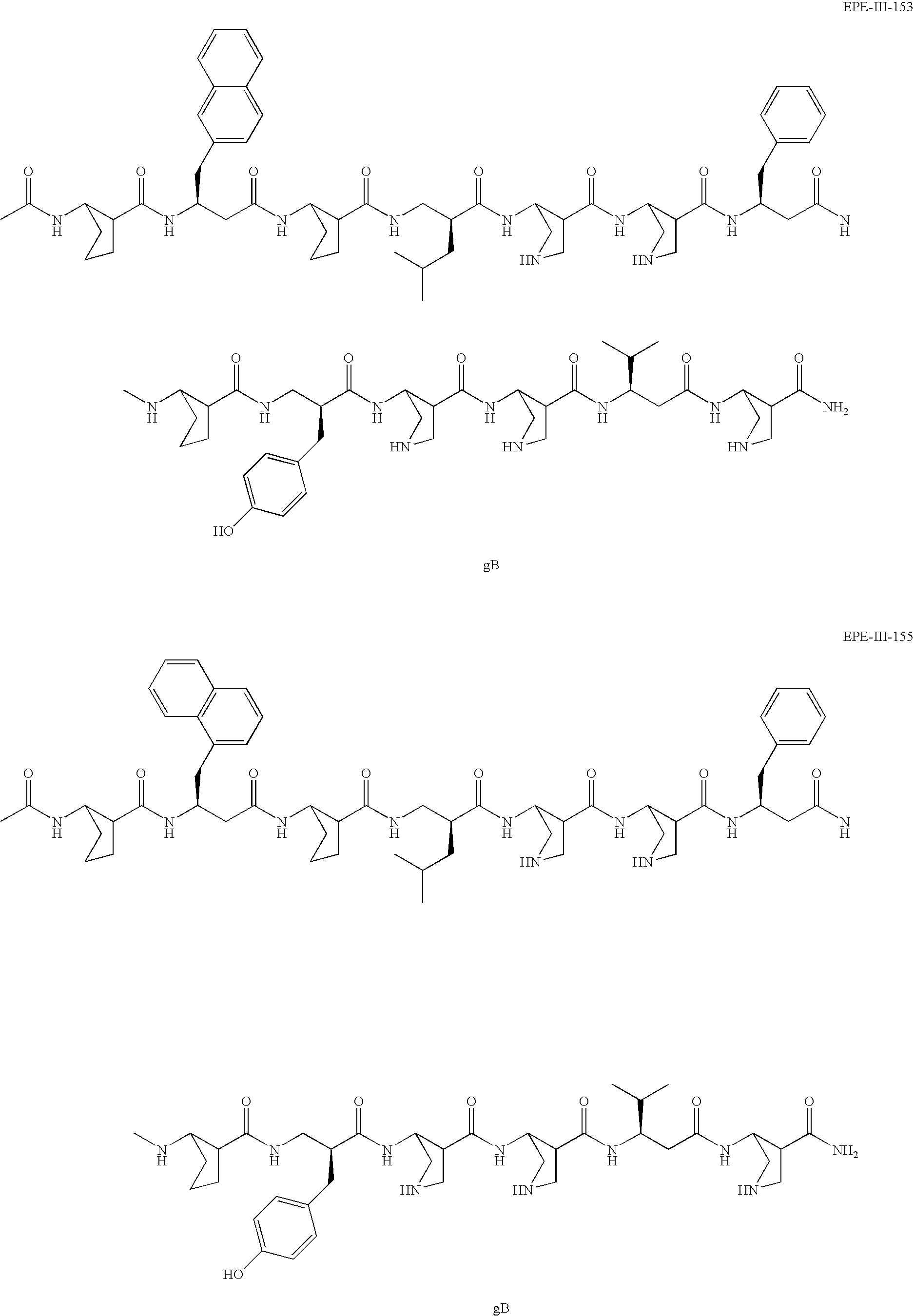

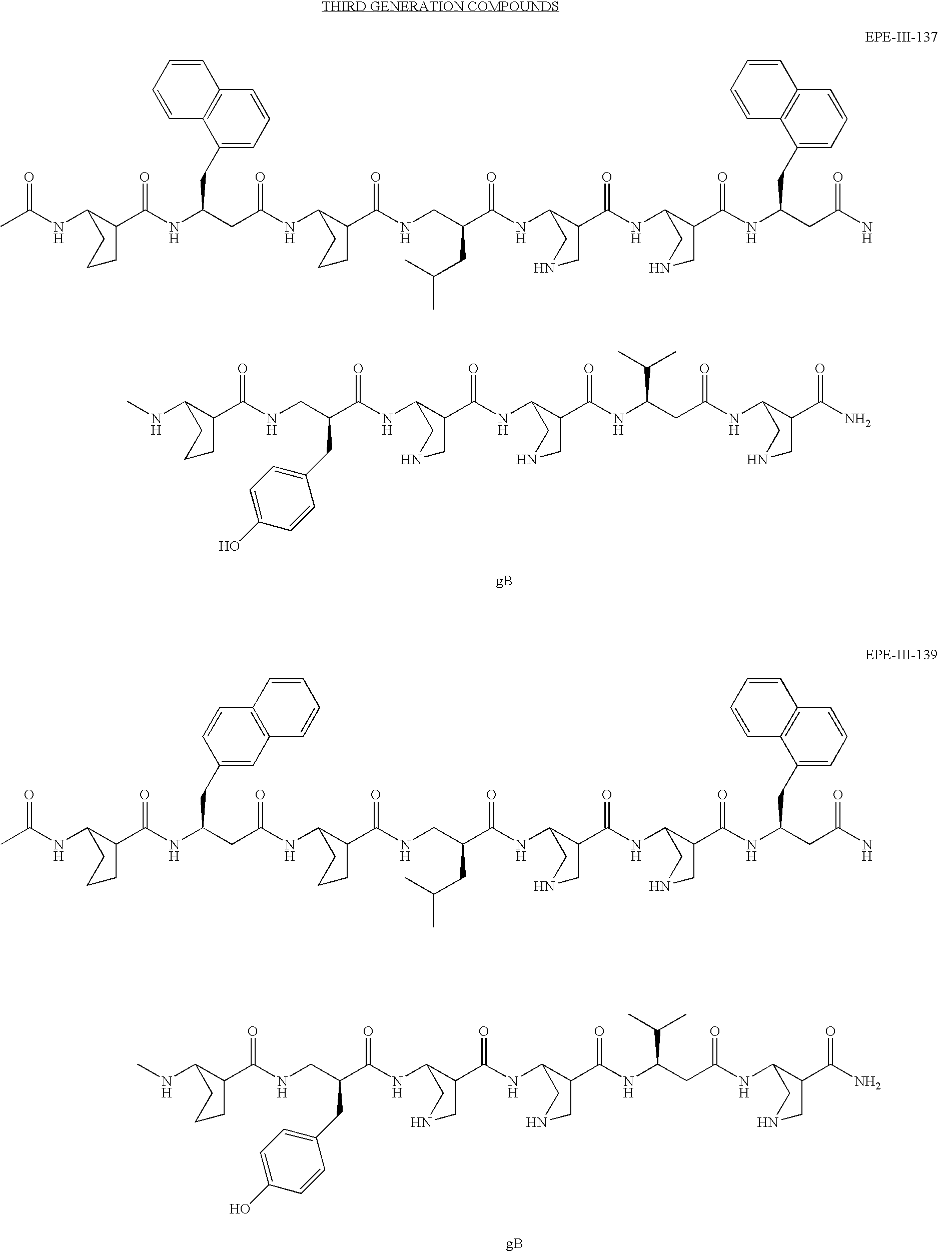

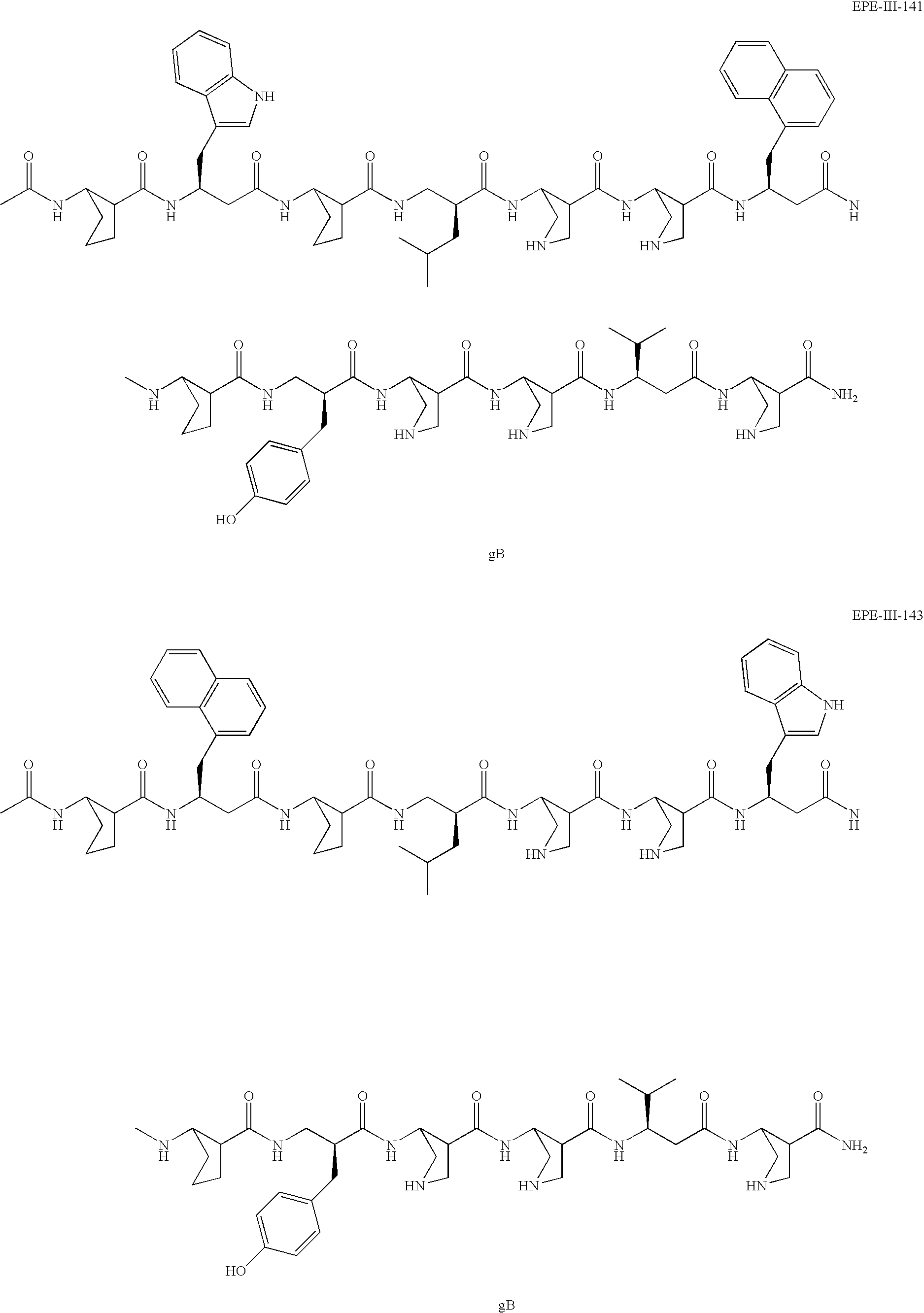

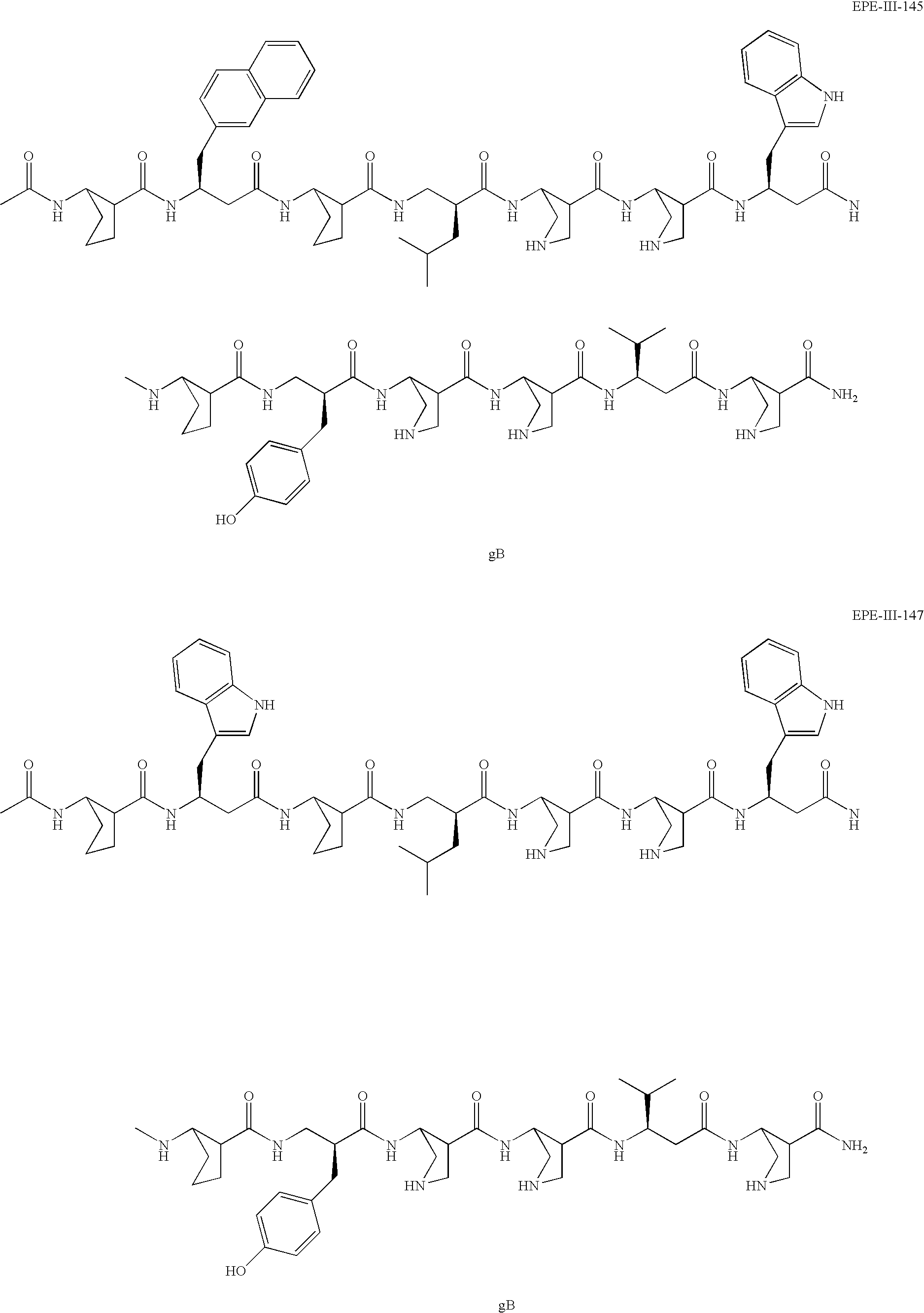

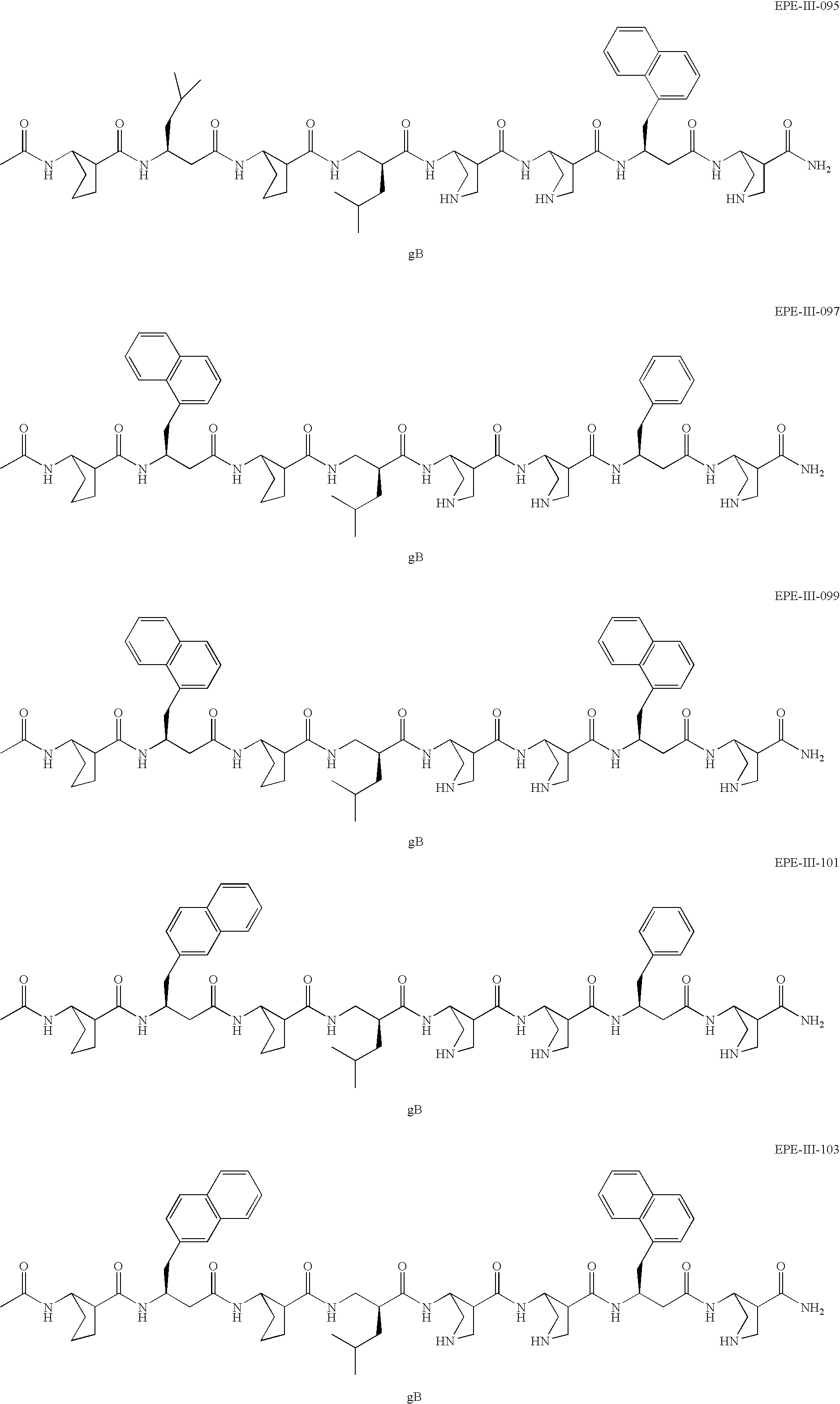

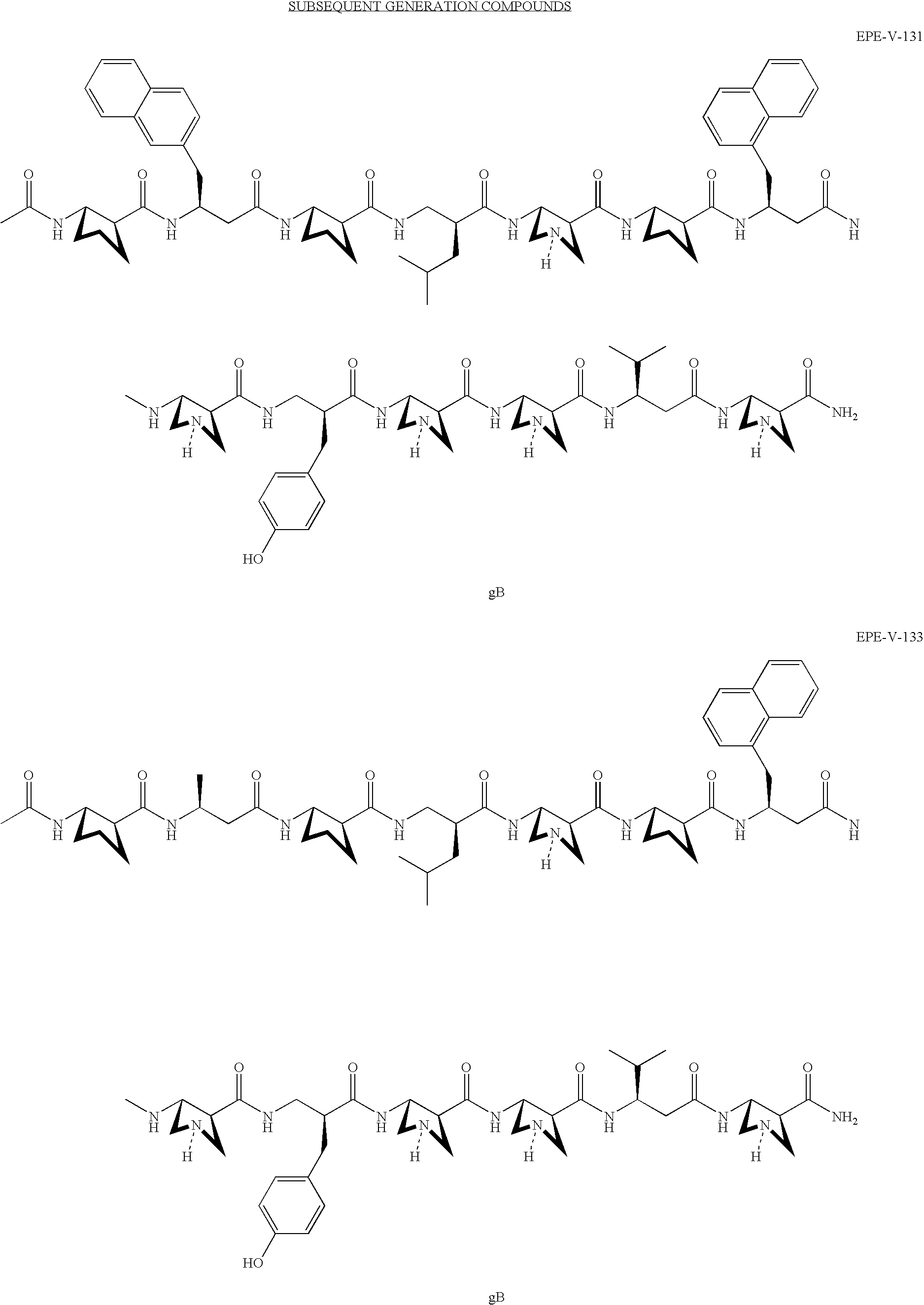

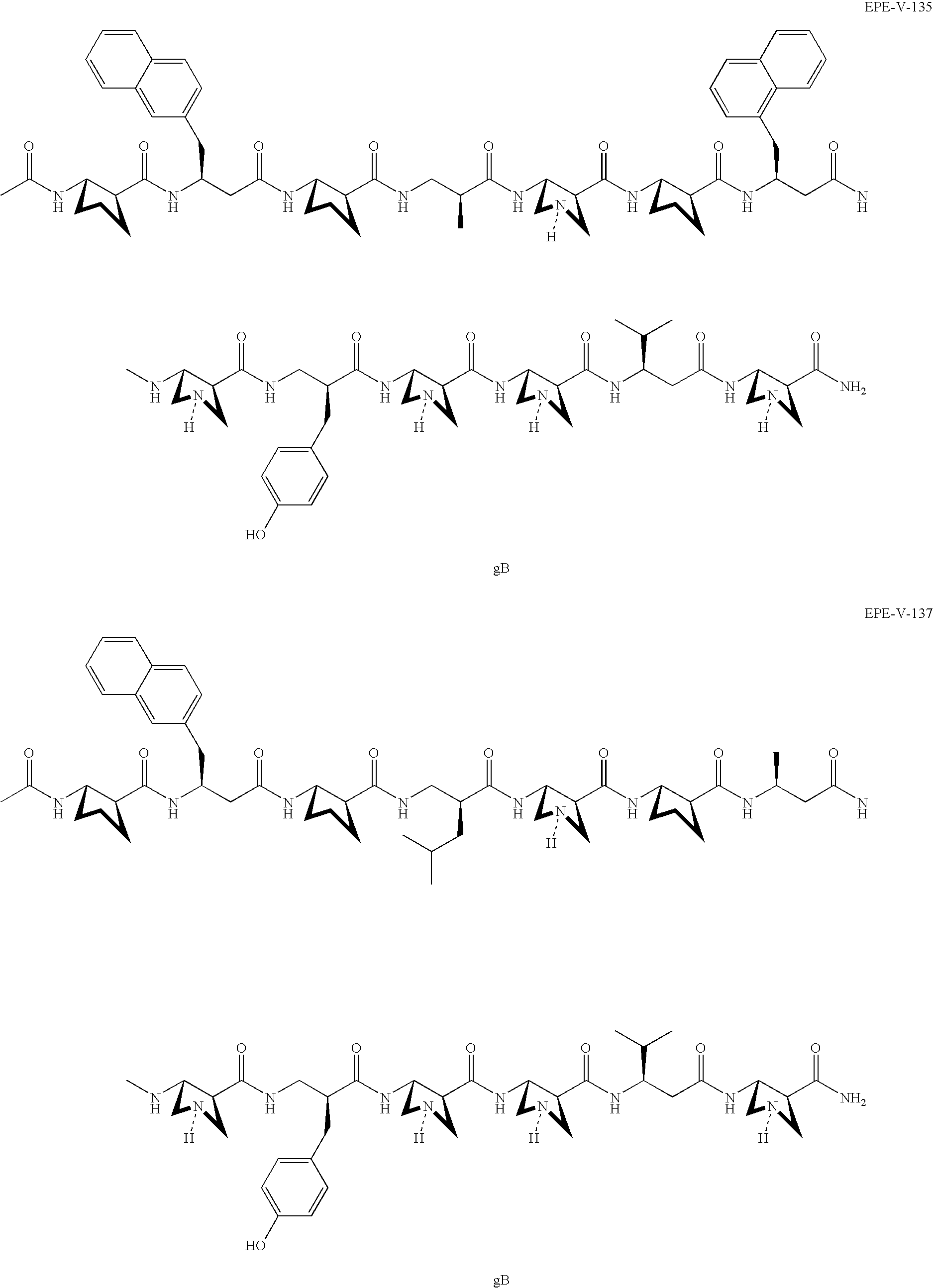

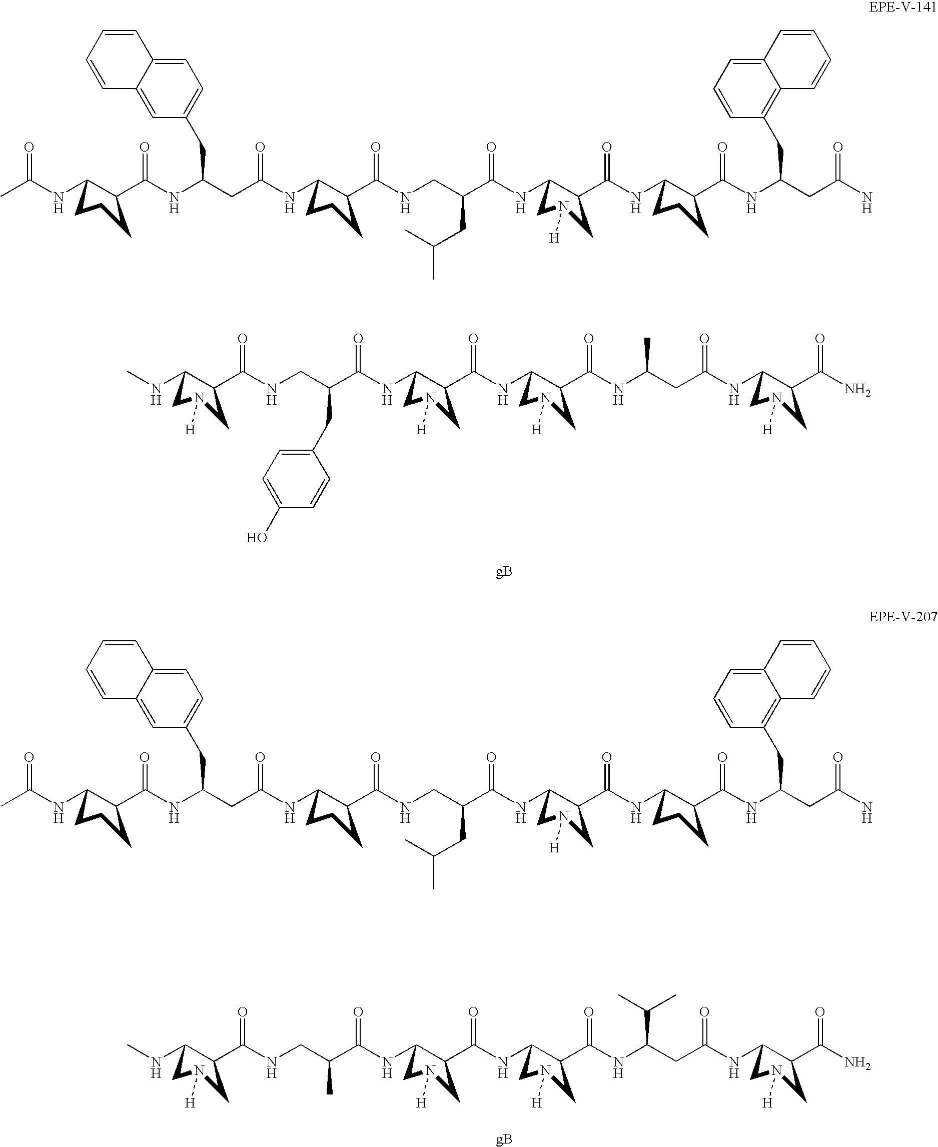

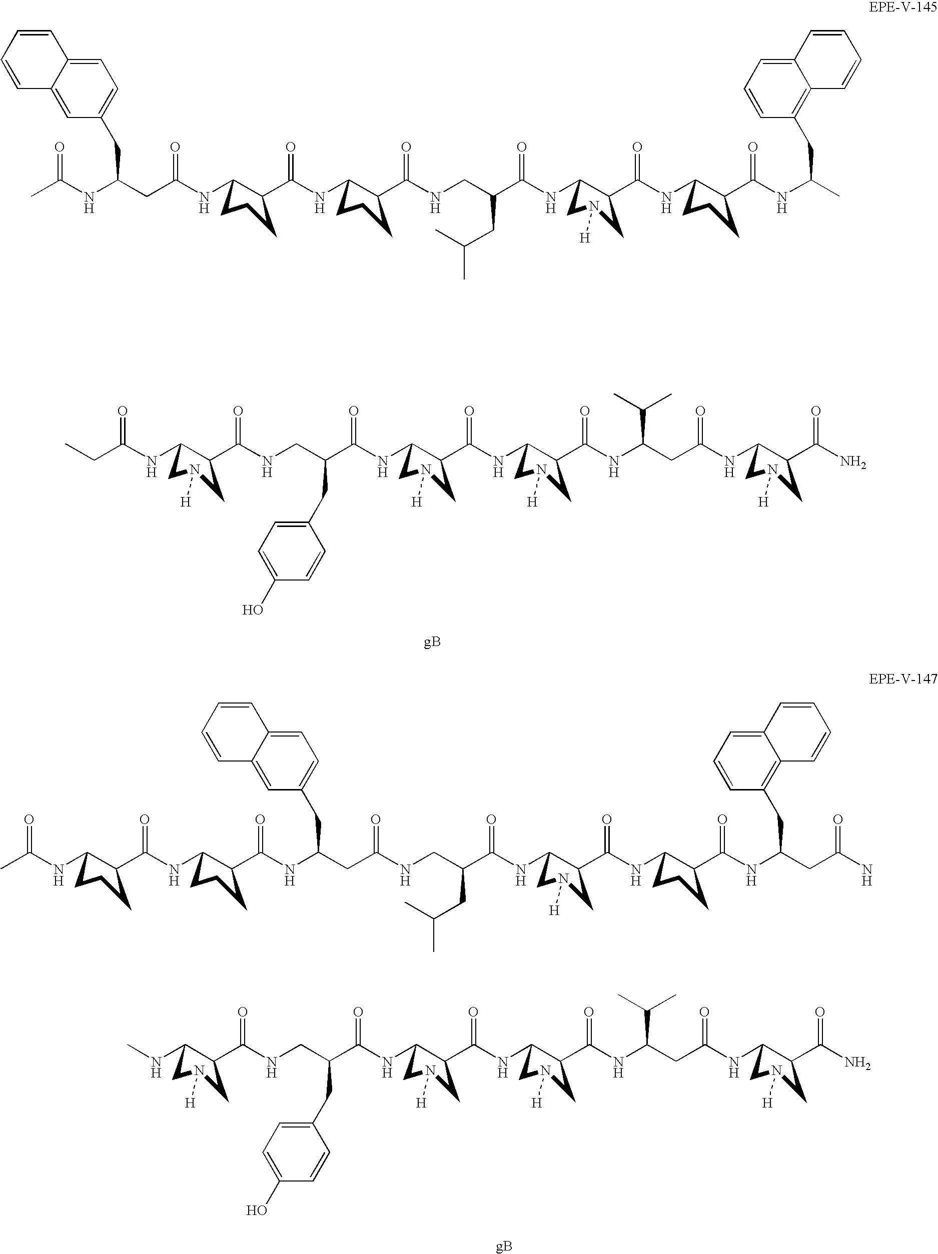

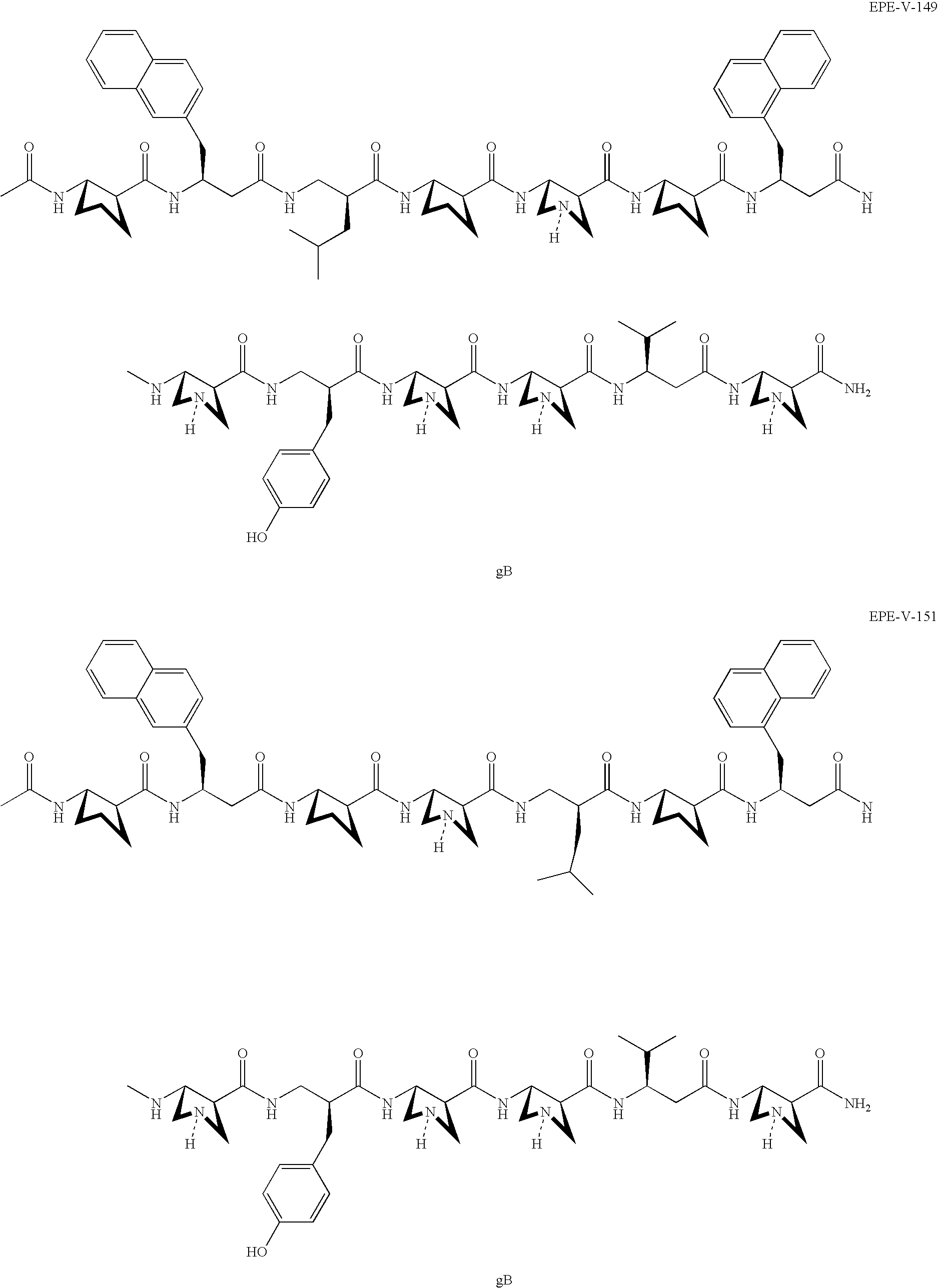

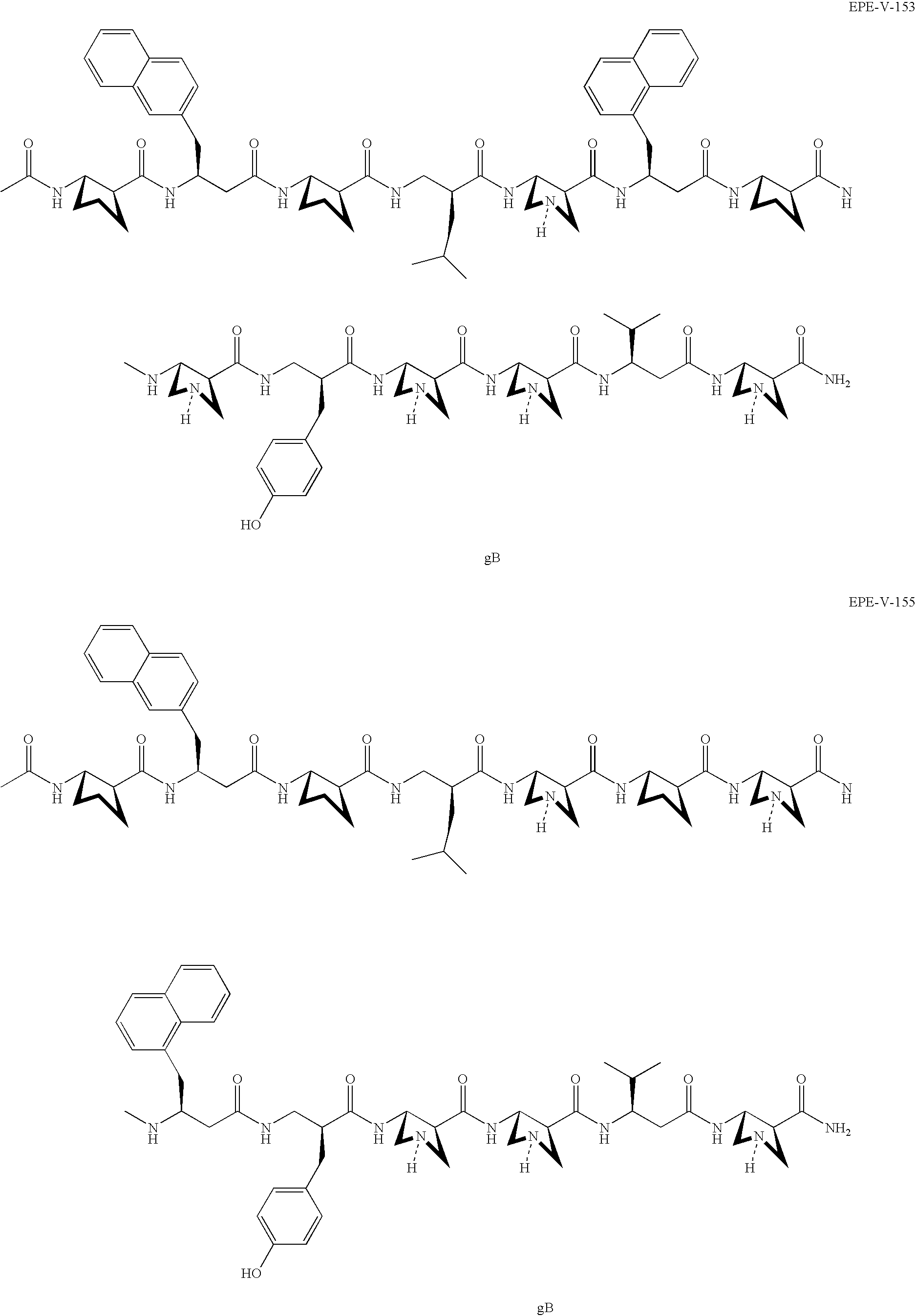

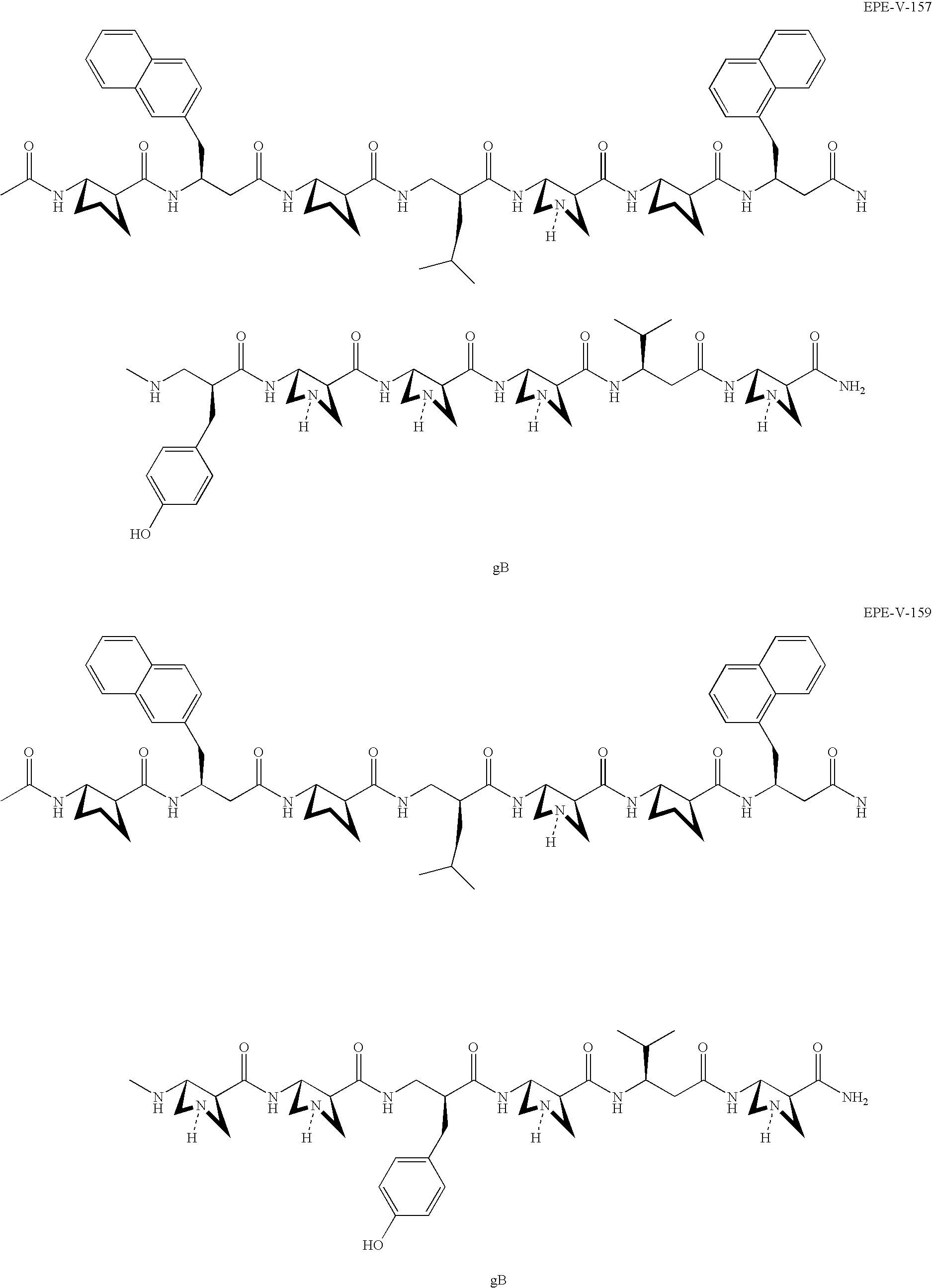

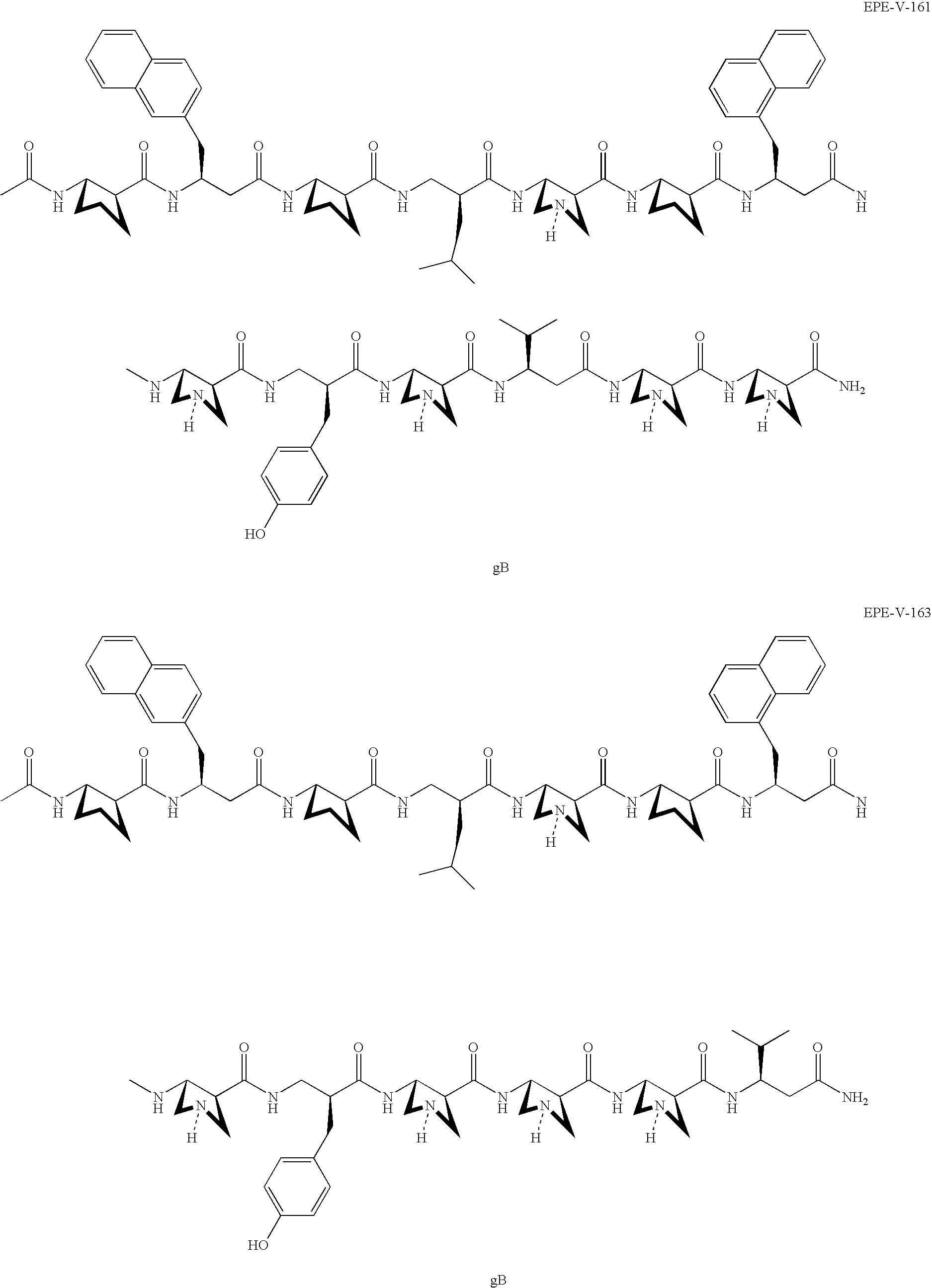

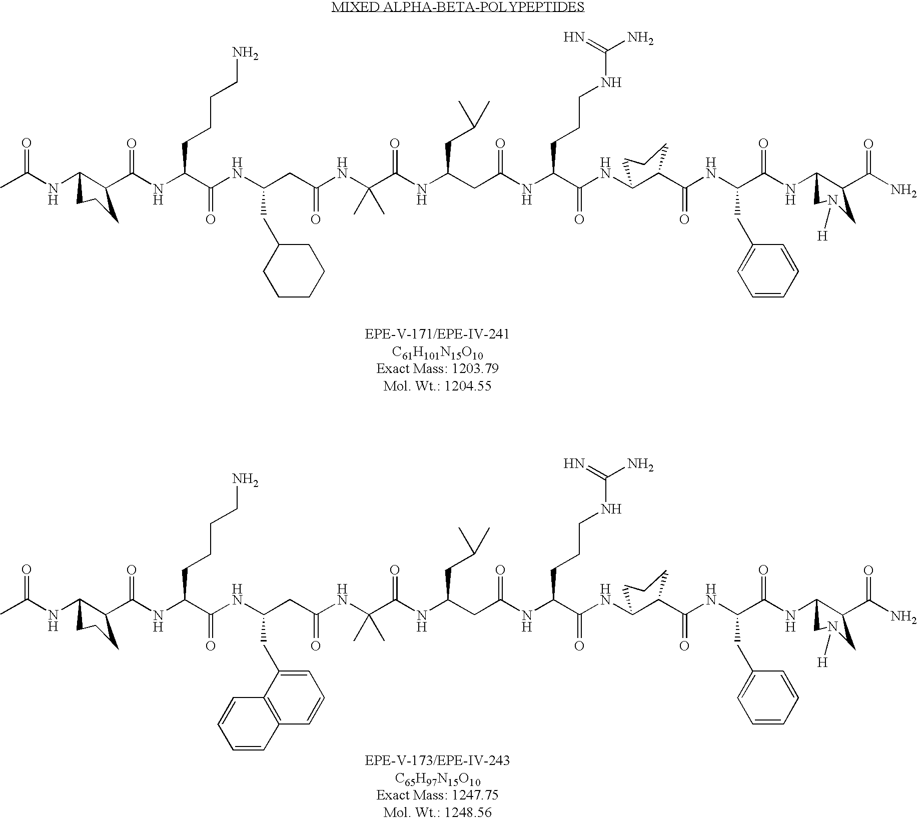

### EXAMPLES

The following Examples are presented to provide a more complete and clear understanding of the invention disclosed and claimed herein. The Examples do not limit the scope of the invention in any fashion.

Cell Lines, Viruses, and Antibodies:

Normal Human Dermal Fibroblast (NHDF) and NIH3T3 cells were cultured in Dulbecco's Modified Eagle Medium (DMEM), supplemented with 10% Fetal Bovine Serum (FBS), 1% L-glutamine, and 1% penicillin-streptomycin. The AD169 strain of HCMV was propagated in NHDFs and purified as previously described (Compton, T. (1993) in J Virol Vol. 67, pp. 3644-3648). HCMV-GFP indicator virus encodes GFP regulated as an immediate early protein and was kindly provided by Deborah H. Spector (University of California, San Diego) (Sanchez, V., Clark, C. L., Yen, J. Y., Dwarakanath, R., and Spector, D. H. (2002) in J Virol Vol. 76, pp. 2973-2989). Murine CMV-EGFP (strain RVG102), with EGFP driven by an immediate early 1/3 promoter was a gift from A. Campbell (Eastern Virginia Medical College, Norfolk); the virus was propagated in NIH3T3 fibroblasts. Herpes simplex virus (HSV-1(KOS)gL86), containing an Escherichia coli lacZ reporter gene, was a generous gift from Rebecca Montgomery (University of Wisconsin, Madison) (Montgomery, R. I., Warner, M. S., Lum, B. J., and Spear, P. G. (1996) in Cell Vol. 87, pp. 427-436); the virus was grown in 79VB4 cells. Vesicular stomatitis virus pseudotyped with G protein and containing a GFP marker (VSV-G), was a kind gift from Yoshihiro Kawaoka (University of Wisconsin, Madison) (Takada, A., Robison, C., Goto, H., Sanchez, A., Murti, K. G., Whitt, M. A., and Kawaoka, Y. (1997) in Proc Natl Acad Sci USA Vol. 94, pp. 14764-14769). Monoclonal antibody against the major tegument phosphoprotein pp65 was purchased from Rumbaugh-Goodwin Institute for Cancer Research, Inc. Alexa Fluor® 488 goat an ti-mouse secondary antibody was purchased from Molecular Probes (Eugene, Oreg.). The 27-78 antibody against glycoprotein B (gB) was a kind gift from William Britt (Schoppel, K., Hassfurther, E., Britt, W., Ohlin, M., Borrebaeck, C. A., and Mach, M. (1996) in Virology Vol. 216, pp. 133-145). The use of polyclonal 6824 antibody against glycoprotein H (gH) was previously described (Huber, M. T., and Compton, T. (1999) in J Virol Vol. 73, pp. 3886-3892). The goat anti-mouse HRP (Horseradish Peroxidase linked) and goat anti-rabbit HRP secondary antibodies were purchased from Pierce Biotechnology (Rockford, Ill).

Beta-Poly Peptides:

The beta-polypeptides and mixed alpha-beta-polypeptides used in the Examples are shown above (first, second, third, and subsequent generations, and mixed alpha-beta-compounds) These compounds were fabricated as described in the Detailed Description.

Virus Entry Assay:

Lyophilized beta-peptides were dissolved in filter-sterilized de-ionized H₂O. The concentration of individual beta-peptides and alpha-peptides was calculated based on absorbance (275 nm) measured with DU® 530 spectrophotometer (Beckman, Fullerton, Calif.). Extinction coefficients were calculated based on information available on the Oregon Medical Laser center web site (http://omlc.ogi.edu/spectra/PhotochemCAD/html/alpha.html.) A precipitate formed upon addition of some beta-peptide stock solutions to cell culture medium while others did not lead to precipitate formation. Because only some beta-peptides displayed precipitation, we concluded that this phenomenon is not related to HCMV entry inhibition. Cells were grown in 12-well plates and infected with the indicated virus (multiplicity of infection (moi)=0.5 pfu/cell). Controls for HCMV-GFP, MCMV-EGFP entry were prepared by pretreating virions with heparin (30 g/ml). To inhibit VSV infection, cells were treated with 30 mM NH₄Cl. For flow cytometric detection of GFP expression, cells were recovered by trypsinization and centrifugation and suspended in PBS and mixed with propidium iodide (Molecular Probes Inc.) as an indicator of cell viability. The samples were analyzed on a FACScan flow cytometer (Becton Dickinson, Mountain View, Calif.) with a standard filter set. The cells were gated for propidium iodide exclusion (live cells) and assayed for GFP content. The data were analyzed using FlowJo (version 6.1, Tree Star Inc., Ashland, Oreg.). Inhibition data were normalized to percent control infection. Active beta-peptides were synthesized independently several times; distinct samples displayed similar activities. For the HSV-1 entry assay, a confluent monolayer of NHDF cells was grown in a 96-well plate and infected with HSV-1(KOS)gL86 as described above. At 6 hr post-infection, the cells were lysed in buffer (100 mM NaH₂PO₄, 10 mM KCl, 1 mM MgSO₄; 0.1% NP-40). O-Nitrophenyl-beta-D-galactopyranoside (ONPG) was added to 2.3 mg/ml and incubated at 25° C. for 6 hr. Then absorbance at 420 nm was measured using SpectraMAXO® 190 spectrophotometer (Molecular Devices, Sunnyvale, Calif.). The assay was set up in quadruplicate and performed three times. The peptide inhibition data was normalized to the level of control infection.

pp65 Translocation Assay:

NHDF cells were grown on glass cover-slips in 12-well plates as above. The HCMV was diluted with 100 M beta-peptide in SF-DMEM and cooled to 4° C. The treatment was then added to cooled cells, which were incubated at 4° C. for 90 min, assuring viral attachment but not entry. The cells were then transferred to 37° C. for 35 min. The cells were then fixed in 3% paraformaldehyde and immunostained for pp65 as described (Lopper, M.; Compton, T. (2004) in J. Virol. Vol. 78, pp. 8333-8341). Images were taken on the Nikon Eclipse TE2000-S with appropriate filters, using consistent exposure times.

### RESULTS

Beta-Peptide Inhibitor Design and Evaluation:

The beta-peptide design effort focused on mimicry of the heptad repeat region previously identified in HCMV gB. No high resolution structural data are available for gB; therefore, an idealized alpha-helical model for the segment to be mimicked was used. The initial target structure was a 12-helical beta-peptide inhibitor that would display along one face a set of side chains matching those thought to contribute to inter-helical interactions of the gB protein, i.e., the nonpolar side chains in gB that have the characteristic coiled-coil spacing (L679, 1682, F686, Y689, and V693) (Lopper, M.; Compton, T. (2004) in J. Virol. Vol. 78, pp. 8333-8341). Formation of the 12-helix requires beta-amino acid residues with a five-membered ring constraint, such as trans-aminocyclopentane carboxylic acid (ACPC) and trans-3-aminopyrrolidine-4-carboxylic acid (APC). (See U.S. Pat. No. 6,613,876.) Placing side chains at specific positions along a 12-helical is most straightforward via acyclic residues that bear a substituent adjacent to the nitrogen atom (³-residues) or adjacent to the carbonyl carbon (²-residues), but these flexible residues diminish 12-helix propensity. (Park, J. S.; Lee, H.-S.; Lai, J. R.; Kim, B. M.; Gelhman, S. H. (2003) in J. Am. Chem. Soc. Vol. 125, pp. 8539-8545.) Therefore, the designs tested in these Examples contain a minimum number of acyclic residues. The APC residues confer not only conformational stability but also water-solubility via the positive charge that develops upon protonation of the ring nitrogen.

A comparative alpha-helical/12 helical net analysis was used to design an initial set of compounds. The alpha-helical net is a flat projection of the alpha-helix that illustrates the spatial relationship among side chain attachment points along the peptide backbone in an alpha-helical conformation (Crick, F. H. C. (1953) in Acta Cryst. Vol. 6, pp. 689-697). Analysis of a heptad repeat sequence reveals a continuous stripe of nonpolar side chains along one side of the alpha-helix; these side chains occupy the first and fourth positions of each heptad repeat. The 12-helix has ^{˜}2.5 residues per turn (Cheng, R. P.; Gellman, S. H.; DeGrado, W. F. (2001) in Chem. Rev. Vol. 101, pp. 3219), and 12-helical net analysis suggests that a stripe of hydrophobic side chains would be created by repeating pentads in which the first and third residues bear nonpolar side chains. Such a 12-helix should display a hydrophobic surface mimicking that of an alpha-helical heptad repeat segment, which, according to the present inventors, could lead to an inhibition of biomolecular processes that require coiled-coil interactions.

Overlaying the alpha-helical and 12-helical nets predicted that a beta-peptide of 13 residues would mimic the heptad repeat segment of gB. This overlay identified side chain positions within a 12-helical beta-peptide that would most closely approximate the set of five key gB side chains as projected from an alpha-helix. There are two possible side chain attachment points in a beta-amino acid residue (² vs. ³), an element of variability that does not exist among alpha-peptides. The helical net overlay clearly predicted the sequence positions for side chain installation along the beta-peptide, but this overlay did not allow distinction among several alternative ²/³ patterns. Empirical tests were used to resolve this issue.

An initial set of isomeric beta-peptides was prepared that differed from one another in ² vs. ³ attachment of side chains intended to mimic L679 and Y689. Four of the side chains on these beta-peptides match perfectly the corresponding gB side chains; synthetic constraints required the use of ²-homoleucine rather than ²-homoisoleucine at the position intended to mimic I682 of gB. These compounds were evaluated for inhibition of HCMV entry in a cell-based infectivity assay. A single compound (ERP-I-123F, see above under “First Generation Compounds”) that blocked HCMV infection was identified. HCMV (moi=0.5 pfu/cell) incubated in the absence of inhibitors resulted in 60% total infected fibroblasts. In the presence of 500 M of ERP-I-123F, the proportion of infected cells was reduced to 20%. No evidence of toxicity could be detected at this high concentration of ERP-I-123F. More detailed analysis revealed an IC₅₀ of ^{˜}300 M for inhibition of HCMV infection by ERP-I-123F (data not shown).

Control experiments were conducted to test the structural hypothesis underlying the beta-peptide design. Replacement of large nonpolar side chains with a methyl group, by substituting ²- or ³-homoalanine at those positions, led to a substantial reduction of anti-HCMV activity. For example, no inhibition of HCMV infection was detected for beta-peptide ERP-I-299 this finding suggests that the hydrophobic side chains of ERP-I-301 are critical for activity. A sequence isomer of ERP-II-005 in which the residues are scrambled was also investigated. In the 12-helical conformation ERP-II-005 does not display the five side chains in a manner that mimics the putative alpha-helical display of gB. Beta-peptide ERP-II-005 proved to be highly toxic toward fibroblasts, in contrast to ERP-I-301, which precluded the examination of ERP-II-005 as a potential negative control compound. The origin of this toxicity is unclear; experiments with human red blood cells (data not shown) indicate that ERP-II-005 does not simply disrupt cell membranes.

A set of 22 second-generation beta-peptides was prepared (structures given above), including fifteen compounds with a single ³-residue change relative to ERP-I-301 and six compounds with two ³-residue changes (see Table 1).

TABLE 1
Normalized inhibition data for second-generation-peptides.
The collective inhibition data illustrate the effect of side-chain
substitutions at positions 2, 7, and 12 on HCMV entry.
Cytotoxicity, as indicated by P1 uptake cited where relevant.

| | | | | Inhibition |
|---|---|---|---|---|
| Peptide | X₂ | X₇ | X₁₂ | (100) % |
| (1) ERP-I-301 | | | | |
| (2) ERP-I-299 | | | | |
| ERP-II-005 | | | | |
| (3) EPE-II-219 | B³-Leu | B³-Phe | B³-Leu | 6.4 |
| (4) EPE-II-221 | B³-Leu | B³-Phe | B³-Ile | 7.5 |
| (5) EPE-II-223 | B³-Leu | B³-Phe | B³-Phe | 10.5 |
| (6) EPE-II-227 | B³-Leu | B³-Phe | B³-1(Nap) | Toxic |
| (7) EPE-II-225 | B³-Leu | B³-Phe | B³-2(Nap) | Toxic |
| (8) EPE-II-229 | B³-Leu | B³-Phe | B³-Tyr | 9.6 |
| (9) EPE-II-233 | B³-Leu | B³-1(Nap) | B³-Val | Toxic |
| (10) EPE-II-231 | B³-Leu | B³-2(Nap) | B³-Val | Toxic |
| (11) EPE-II-235 | B³-Leu | B³-Tyr | B³-Val | 13.2 |
| (12) EPE-II-237 | B³-Leu | B³-Leu | B³-Val | 20.2 |
| (13) EPE-II-239 | B³-Phe | B³-Phe | B³-Val | 22.1 |
| (14) EPE-II-241 | B³-2(Nap) | B³-Phe | B³-Val | 52.2 |
| (15) EPE-II-243 | B³-1(Nap) | B³-Phe | B³-Val | 63.8 |
| (16) EPE-II-247 | B³-Tyr | B³-Phe | B³-Val | 6.9 |
| (17) EPE-II-245 | B³-Ile | B³-Phe | B³-Val | 6.8 |
| (18) EPE-III-137 | B³-1(Nap) | B³-1(Nap) | B³-Val | 77.9 |
| (19) EPE-III-139 | B³-2(Nap) | B³-1(Nap) | B³-Val | 93.4 |
| (20) EPE-III-141 | B³-Trp | B³-1(Nap) | B³-Val | 73.2 |
| (21) EPE-III-143 | B³-1(Nap) | B³-Trp | B³-Val | 29.7 |
| (22) EPE-III-145 | B³-2(Nap) | B³-Trp | B³-Val | 25.5 |
| (23) EPE-III-147 | B³-Trp | B³-Trp | B³-Val | 26.1 |

Data here are presented as percent inhibition.

Several of these newer compounds were significantly more active than ERP-I-301. The trends indicate that large, aromatic chains at position 2 or at both positions 2 and 7 enhance fusion inhibition, while, curiously, placement of such side chains at position 7 alone or position 12 alone leads to fibroblast toxicity. Three of these beta-peptides were selected for further analysis (Compounds EPE-II-219, EPE-II-247, and EPE-III-139). Dose-response experiments demonstrated that the most active beta-peptide inhibitor, EPE-III-139, had an IC₅₀ of ^{˜}30 M in the infectivity assay, a ten-fold improvement over the activity of ERP-I-301. At 100 M, beta-peptide EPE-III-139 allowed only ca. 10% infection; in stark contrast, the alpha-peptide segments derived from gB are inactive at 100 M.

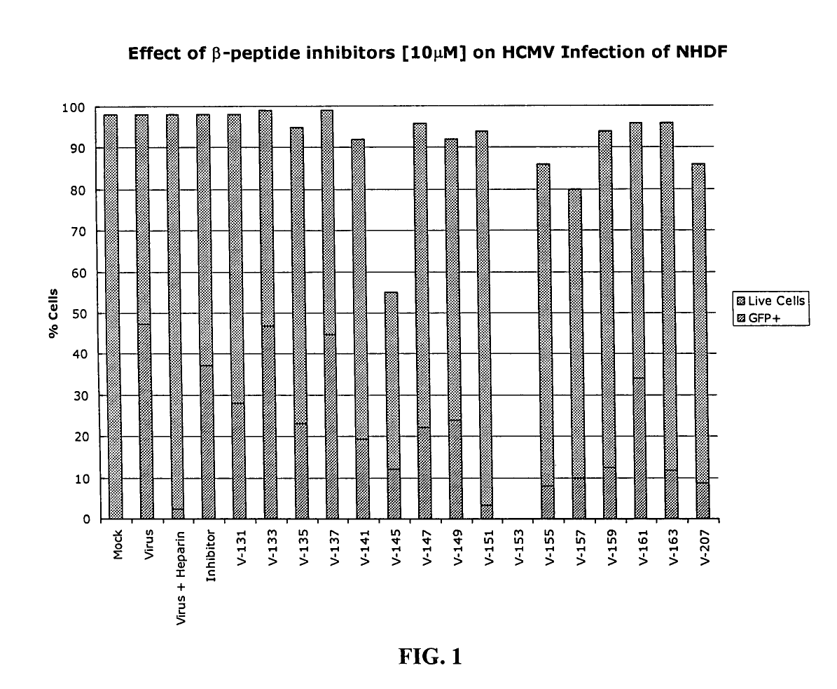

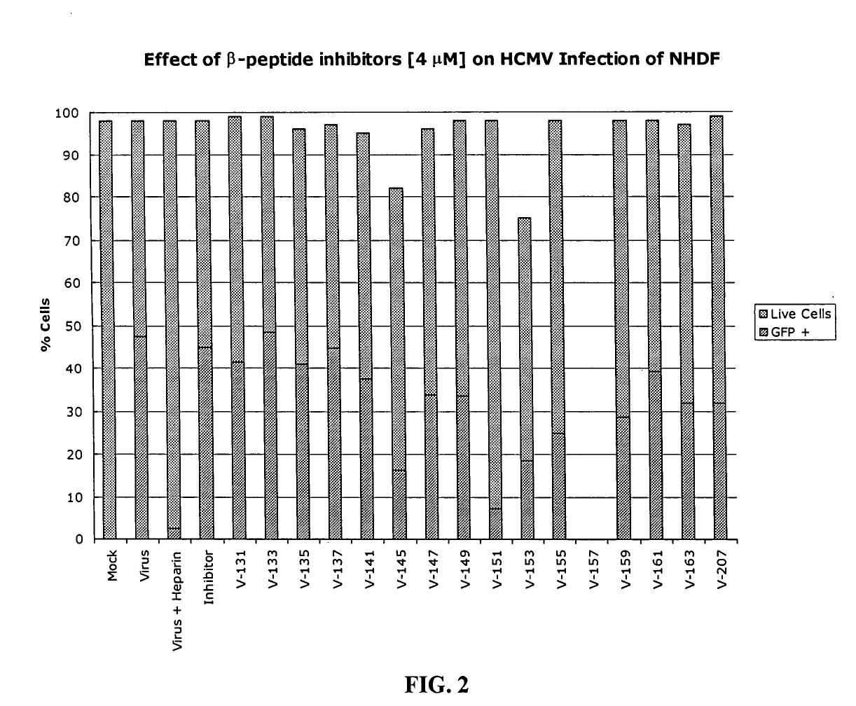

The results of the HCMV infectivity assays (using NHDF cells) are presented in FIGS. 1 and 2. FIG. 1 shows the results for each compound when administered at a concentration of 10 M; FIG. 2 shows the results for each compound when administered at a concentration of 4 M. In both of FIGS. 1 and 2, the entire height of each bar represents the percentage of live cells remaining after being treated with each compound; the area below the horizontal line in each bar represents the percentage of GFP-positive cells. In both figures, the compound identified as “inhibitor” is compound EPE-III-139.

The data presented in Tables 1 and 2 clearly show that beta-polypeptides of the type described herein have biological activity to inhibit viral infection of mammalian cells in general, and to prevent viral infection of human cells, and to prevent HCMV infection of NHDF cells in particular. Thus, the compounds disclosed herein can be used in a method to inhibit the viral infection of mammalian cells. The compounds can also be formulated into pharmaceutical compositions to inhibit viral infection of mammalian cells.

Beta-Peptide Inhibitors Target Membrane Fusion:

The infectivity assays used in these Examples measures immediate early gene expression; immediate early (IE) proteins are the first viral proteins expressed in infected cells. Inhibition of viral gene expression could reflect interference at a variety of points in the virus life cycle such as inhibition of IE gene transcription or translation. A virion content delivery assay was performed to test whether the beta-peptides act at the viral entry stage, as they have been designed to do. Immediately upon membrane fusion, the phosphoprotein-rich tegument layer of the virus is released into the cytoplasm of the target cell. The pp65 protein, highly abundant in the virion tegument, diffuses rapidly to the nucleus after membrane fusion. Thus, nuclear localization of pp65 can be used to assess membrane fusion activity and rule out alternative mechanisms of beta-peptide action. As in the infectivity assays, exposure of fibroblasts to soluble heparin serves as a positive control for viral entry inhibition: this treatment eliminates pp65 accumulation in the nucleus. Similarly, the most potent beta-peptide inhibitor, EPE-III-139, blocked nuclear localization of pp65, while inactive beta-peptides ERP-I-301 and EPE-II-219 had no effect on pp65 uptake. While not being bound to any specific underlying mechanism, this observation indicates that the active beta-peptides inhibit HCMV infection at the level of virus-cell membrane fusion.

Significance of the Examples:

Again, while not being bound to any underlying biological mechanism, the results of the Examples suggest that beta-peptides inhibit HCMV entry into target cells by interacting with viral fusion machinery. It is proposed that this inhibitory effect arises from the beta-peptides' adoption of a folded conformation, the 12-helix, which generates a specific side chain arrangement that allows recognition of at least one target protein. Two-dimensional NMR data (data not shown) for ERP-I-301 indicate a substantial 12-helical propensity. The present hypothesis to explain HCMV fusion inhibition is based on the assumption that entry requires the gB protein, on the virion surface, to be initially triggered to adopt a fusion-active conformation by interaction with cellular receptors. It is further assumed that the heptad repeat segment of gB is exposed in the fusion-active conformation, and that this segment must associate with the heptad repeat segments of other fusion-active gB protein molecules and/or with the heptad repeat segment of gH in order for fusion of the viral envelope with the cell membrane to proceed. It is proposed that the beta-peptide binds to the heptad repeat segment of gB in the fusion-active conformation, blocking homo- and/or hetero-protein-protein associations required for fusion. Because no structural information is yet available for gB or other HCMV glycoproteins, the beta-peptide inhibitors described herein are useful both to prevent HCMV infection and also as research tools to elucidate the fusion mechanism.

The beta-peptides have the further advantage, relative to alpha-peptide inhibitors, of resistance to proteolytic degradation. The Examples provide evidence of foldamer-based inhibition of HCMV entry, thus indicating that these compounds are useful to inhibit and to treat viral infection of mammals, including humans.

The significance of the foldamer-based approach for generating inhibitors of HCMV fusion described herein is highlighted by the very poor inhibitory activity observed for alpha-peptides derived from HCMV proteins gB and gH. The inadequacy of alpha-peptide inhibitors of HCMV and HSV entry suggests that a more sophisticated strategy will be required for development of fusion inhibitors effective against herpesviruses and other refractory pathogenic viruses. The ready application of combinatorial synthesis methods to beta-peptides and other foldamers also facilitates the fabrication of a wide array of distinct compounds.

What is claimed is:

1. A method for inhibiting viral entry into an animal host cell, the method comprising administering to the host cell a viral fusion-inhibiting amount of a compound capable of inhibiting viral entry into the host cell, wherein the compound is selected from the group consisting of beta-amino acid-containing polypeptides comprising eight (8) or more residues, wherein at least one of the residues is a beta-amino acid residue wherein the alpha and beta carbons are cyclically constrained, and pharmaceutically suitable salts thereof.

2. The method of claim 1, wherein at least three (3) of the residues are beta-amino acid residues wherein the alpha and beta carbons are cyclically constrained.

3. The method of claim 1, wherein at least five (5) of the residues are beta-amino acid residues wherein the alpha and beta carbons are cyclically constrained.

4. The method of claim 1, wherein the compound is selected from the group consisting of: ERP-I-301, EPE-II-219, EPE-II-221, EPE-II-223, EPE-II-227, EPE-II-225, EPE-II-229, EPE-II-233, EPE-II-231, EPE-II-235, EPE-II-237, EPE-II-239, EPE-II-241, EPE-II-243, EPE-II-247, EPE-II-245, EPE III-137, EPE-III-139, EPE-III-141, EPE-III-143, EPE-III-145, EPE-III-147, and pharmaceutically suitable salts thereof.

5. The method of claim 1, wherein the compound is selected from the group consisting of beta-amino acid-containing polypeptides comprising eight (8) to thirteen (13) residues, all of which are beta-amino acid residues, and wherein at least one of the residues is a beta-amino acid residue wherein the alpha and beta carbons are cyclically constrained, and pharmaceutically suitable salts thereof.

6. The method of claim 1, wherein the compound is selected from the group consisting of beta-amino acid-containing polypeptides comprising eight (8) to thirteen (13) residues, wherein the polypeptide comprises at least one alpha-amino acid residue, and wherein at least one other of the residues is a beta-amino acid residue wherein the alpha and beta carbons are cyclically constrained, and pharmaceutically suitable salts thereof.

7. The method of claim 6, wherein the compound is selected from the group consisting of: 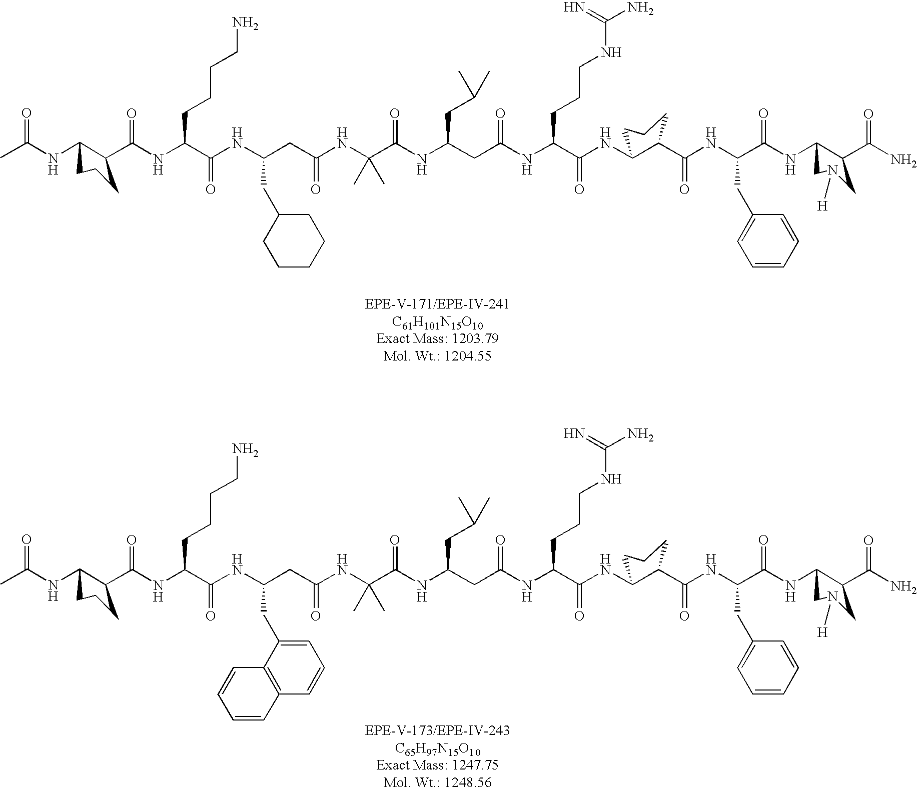
and pharmaceutically suitable salts thereof.

8. The method of claim 1, wherein the compound is administered in combination with a pharmaceutically suitable carrier suitable for a delivery route selected from the group consisting of oral, parenteral, topical, subcutaneous, transdermal, intramuscular, intravenous, intra-arterial, buccal, and rectal.

9. A pharmaceutical composition for inhibiting viral infection in mammalian cells, the composition comprising, a viral fusion-inhibiting amount of a compound capable of inhibiting viral entry into the host cell, wherein the compound is selected from the group consisting of beta-amino acid-containing polypeptides comprising eight (8) or more residues, wherein at least one of the residues is a beta-amino acid residue wherein the alpha and beta carbons are cyclically constrained, and pharmaceutically suitable salts thereof.

10. The pharmaceutical composition of claim 9, wherein at least three (3) of the residues are beta-amino acid residues wherein the alpha and beta carbons are cyclically constrained.

11. The pharmaceutical composition of claim 9, wherein at least five (5) of the residues are beta-amino acid residues wherein the alpha and beta carbons are cyclically constrained.

12. The pharmaceutical composition of claim 9, wherein the compound is selected from the group consisting of: ERP-I-301, EPE-II-219, EPE-II-221, EPE-II-223, EPE-II-227, EPE-II-225, EPE-II-229, EPE-II-233, EPE-II-231, EPE-II-235, EPE-II-237, EPE-II-239, EPE-II-241, EPE-II-243, EPE-II-247, EPE-II-245, EPE III-137, EPE-III-139, EPE-III-141, EPE-III-143, EPE-III-145, EPE-III-147, and pharmaceutically suitable salts thereof.

13. The pharmaceutical composition of claim 9, wherein the compound is selected from the group consisting of beta-amino acid-containing polypeptides comprising eight (8) to thirteen (13) residues, all of which are beta-amino acid residues, and wherein at least one of the residues is a beta-amino acid residue wherein the alpha and beta carbons are cyclically constrained, and pharmaceutically suitable salts thereof.

14. The pharmaceutical composition of claim 9, wherein the compound is selected from the group consisting of beta-amino acid-containing polypeptides comprising eight (8) to thirteen (13) residues, wherein the polypeptide comprises at least one alpha-amino acid residue, and wherein at least one other of the residues is a beta-amino acid residue wherein the alpha and beta carbons are cyclically constrained, and pharmaceutically suitable salts thereof.

15. The pharmaceutical composition of claim 14, wherein the compound is selected from the group consisting of: 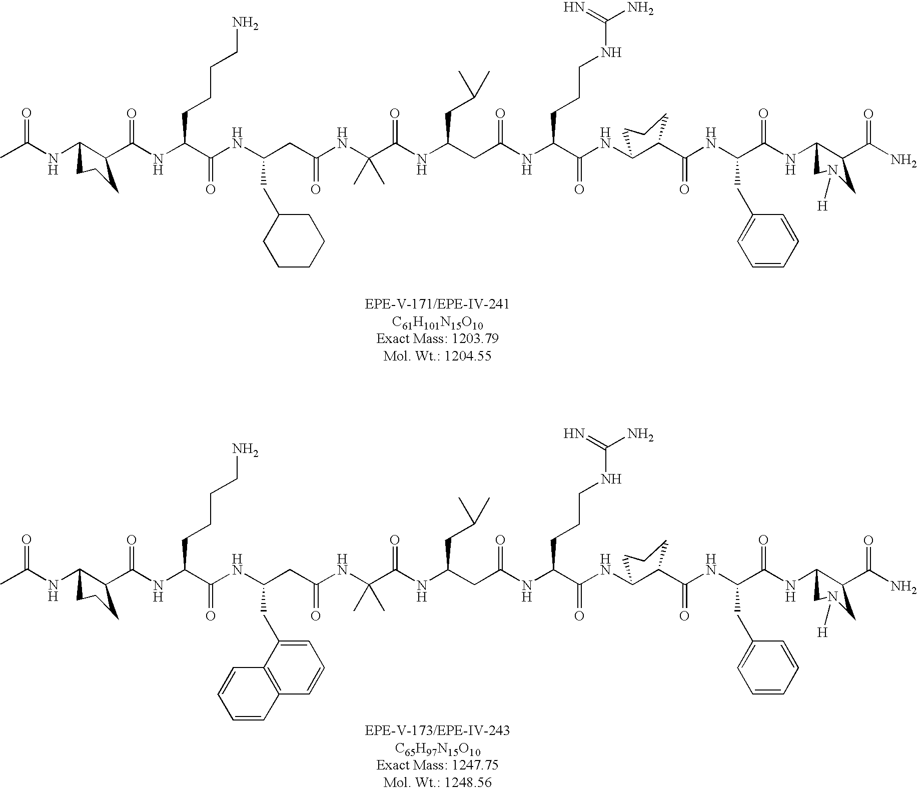
and pharmaceutically suitable salts thereof.

16. The pharmaceutical composition of claim 9, further comprising, in combination, a pharmaceutically suitable carrier suitable for a delivery route selected from the group consisting of oral, parenteral, topical, subcutaneous, transdermal, intramuscular, intravenous, intra-arterial, buccal, and rectal.
